(19) United States
(12) Patent Application Publication
(10) Pub. No.: US 2012/0022058 A1
(43) Pub. Date: Jan. 26, 2012

(54) 4,5-DIHYDRO-1H-PYRAZOLE COMPOUNDS AND THEIR PHARMACEUTICAL USES

(75) Inventors: Graciela Barbieri Arhancet, Creve Coeur,, MO (US); Agustin Casimiro-Garcia, Mystic, CT (US); Xiangyang Chen, Chesterfield, MO (US); David Hepworth, Noank, CT (US); Marvin Jay Meyers, Chesterfield, MO (US); David Walter Piotrowski, Waterford, CT (US); Raj Kumar Raheja, La Jolla, CA (US)

(73) Assignee: Pfizer Inc.

(21) Appl. No.: 13/262,854

(22) PCT Filed: Mar. 26, 2010
(86) PCT No.: PCT/IB2010/051337

§ 371 Date: Oct. 4, 2011

Related U.S. Application Data
(60) Provisional application No. 61/168,351, filed on Apr. 10, 2009.

(51) Int. Cl. A61K 31/4439; C07D 401/14; A61K 31/444; A61K 31/517; C07D 413/10; A61K 31/538; A61K 31/4709; A61P 9/00; A61P 13/12; A61P 1/16; A61P 29/00; A61P 25/00; A61P 5/48; A61P 3/10; C07D 401/04

(52) U.S. Cl. 514/230.5; 546/275.4; 514/341; 546/256; 514/333; 544/284; 514/266.21; 544/105; 514/339; 546/158; 514/312; 544/92

(57) ABSTRACT

Mineralocorticoid receptor antagonists (MRa), pharmaceutical compositions containing such inhibitors and the use of such inhibitors to treat, for example, diabetic nephropathy and hypertension in mammals, including humans.

### BACKGROUND OF THE INVENTION

This invention relates to compounds that are mineralocorticoid receptor antagonists (MRa) pharmaceutical compositions containing such antagonists and the use of such inhibitors to treat for example, diabetic nephropathy and hypertension.

Hypertension affects about 20% of the adult population in developed countries. In the adult population aged 60 years or older, this percentage increases to about 60% to 70%. Hypertension also is associated with an increased risk of other physiological complications including stroke, myocardial infarction, atrial fibrillation, heart failure, peripheral vascular disease and renal impairment. Although a number of anti-hypertensive drugs are available in various pharmacological categories, the efficacy and safety of such drugs can vary from patient to patient. There are a variety of physiological conditions associated with hypertension and one exemplary condition is diabetic nephropathy.

Mineralocorticoid receptor antagonists are one class of drugs that can be used to treat hypertension and/or related physiological complications (Jewell, C. W., et al., Cardiovascular & Hematological Agents in Medicinal Chemistry (2006) Vol. 4, pgs. 129-153). Mineralocorticoids, such as aldosterone, are involved in regulating salt and water balance in mammals. Activation of the mineralocorticoid receptor can induce hypertension and cause other detrimental cardiovascular and physiological effects. Two mineralocorticoid receptor antagonists, spironolactone (ALDACTONE™) and eplerenone (INSPRA™), are presently available and indicated for the treatment of hypertension and heart failure (Baxter, J. D., et al., Molecular and Cellular Endocrinology (2004) Vol. 217, pgs. 151-165). WO 2008/053300 describes certain pyrazoline compounds as mineralocorticoid receptor antagonists.

WO 03/079973 describes certain 4,5-dihydropyrazole derivatives as mitotic kinesins.

The present invention is particularly directed to mineralocorticoid receptor antagonists that are non-steroidal compounds. Use of a non-steroidal mineralocorticoid receptor antagonist potentially provides certain advantages over a steroidal mineralocorticoid receptor antagonist including, e.g., further improvement in selectivity with respect to the sex hormone receptors; less complex and costly chemical synthesis; and the like.

There remains a need for pharmaceutical agents that have MRa activity and are useful in the treatment, prevention or diminution of the manifestations of the maladies described herein.

### SUMMARY OF THE INVENTION

The present invention is directed to a compound of the Formula I

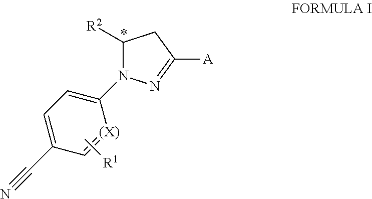

a prodrug thereof, or a pharmaceutically acceptable salt of said compound or of said prodrug;

### X is N or C;

### A is

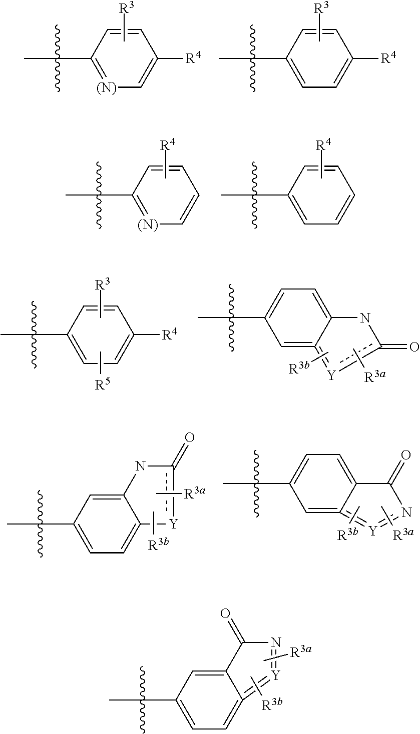

R¹ is H, halo, cyano, (C₁-C₄)alkylthio, (C₁-C₄)alkoxy or (C₁-C₄)alkyl, said (C₁-C₄)alkylthio, (C₁-C₄)alkoxy or (C₁-C₄)alkyl optionally substituted with one to nine fluoros;
R² is cyclo(C₃-C₆)alkyl, said cyclo(C₃-C₆)alkyl optionally substituted with one to four fluoros;
R³ is H, halo, hydroxyl, carboxy, carbamoyl, (C₁-C₄)alkyl, cyclo(C₃-C₆)alkyl, (C₁-C₄)alkylamino, (C₁-C₄)alkoxy, (C₁-C₄)alkylthio, (C₁-C₄)alkoxycarbonyl, (C₁-C₄)alkylsulfonyl, aminosulfonyl, (C₁-C₄)alkylsulfonylamino, (C₁-C₄)alkylcarbamoyloxy, mono-N— or di-N—,N—(C₁-C₄)alkylaminosulfonyl, mono-N— or di-N—,N—(C₁-C₄)alkylaminocarbonyl or (C₁-C₄)alkylcarbonylamino, said (C₁-C₄)alkyl optionally mono-substituted with hydroxyl, cyano, carboxy, or carbamoyl;
R⁴ is halo, hydroxyl, carboxy, carbamoyl, (C₁-C₄)alkyl, cyclo(C₃-C₆)alkyl, (C₁-C₄)alkylamino, (C₁-C₄)alkoxy, (C₁-C₄)alkylthio, (C₁-C₄)alkoxycarbonyl, (C₁-C₄)alkylsulfonyl, aminosulfonyl, (C₁-C₄)alkylsulfonylamino, (C₁-C₄)alkylcarbamoyloxy, mono-N— or di-N—,N—(C₁-C₄)alkylaminosulfonyl, mono-N— or di-N—,N—(C₁-C₄)alkylaminocarbonyl, (C₁-C₄)alkylcarbonylamino, cyano, tetrazolylcarbamoyl, (C₁-C₄)alkoxycarbonyl(C₁-C₄)alkyl, (C₁-C₄)alkoxycarbonyl, (C₁-C₄)alkylsulfonylaminocarbonyl or said (C₁-C₄)alkyl optionally mono-substituted with hydroxyl, cyano, carboxy, or carbamoyl and said mono-N— or di-N—,N—(C₁-C₄)alkylaminocarbonyl optionally mono-substituted on said (C₁-C₄)alkyl with hydroxyl, cyano or carboxy;
R⁵ is H, halo or (C₁-C₄)alkyl;
Y is a unsaturated, partially saturated or fully saturated one to three membered straight carbon chain, wherein the carbons may optionally be replaced with one or two heteroatoms selected independently from oxygen, sulfur and nitrogen, to form a five to seven membered ring; and
R^{3a} or R^{3b} is H or (C₁-C₄)alkyl,
wherein at least X is N or the A substituent contains a ring nitrogen.

Yet another aspect of this invention is directed to a method for treating cardiovascular conditions, renal conditions, liver conditions, inflammatory conditions, pain, retinopathy, neuropathy, insulinopathy, diabetic nephropathy, edema, endothelial dysfunction or baroreceptor dysfunction in a mammal (including a human being either male or female) by administering to a mammal in need of such treatment a cardiovascular conditions, renal conditions, liver conditions, inflammatory conditions, pain, retinopathy, neuropathy, insulinopathy, diabetic nephropathy, edema, endothelial dysfunction or baroreceptor dysfunction treating amount of a compound of Formula I, a prodrug thereof, or a pharmaceutically acceptable salt of said compound or of said prodrug. A preferred method is wherein diabetic nephropathy is treated.

Also provided herein are compositions comprising a pharmaceutically effective amount of one or more of the compounds described herein and a pharmaceutically acceptable vehicle, carrier or excipient.

This invention is also directed to pharmaceutical combination compositions comprising: a therapeutically effective amount of a composition comprising

a first compound, said first compound being a Formula I compound, a prodrug thereof, or a pharmaceutically acceptable salt of said compound or of said prodrug;

a second compound, said second compound being an anti-hypertensive agent; and/or optionally

a pharmaceutical vehicle, diluent or carrier.

Preferably the second compound is a loop diuretic and it is especially preferred that it is torsemide.

All patents and patent applications referred to herein are hereby incorporated by reference.

Other features and advantages of this invention will be apparent from this specification and the appendant claims which describe the invention.

### BRIEF DESCRIPTION OF THE DRAWINGS

FIG. 1 is an X-ray crystal structure for (R)-6-(3-chloro-5-cyclopentyl-4,5-dihydro-1H-pyrazol-1-yl)-2-methylnicotinonitrile.

FIG. 2 is a characteristic x-ray powder diffraction pattern showing a crystalline form of Example 4, (R)-6-(1-(4-cyano-3-methylphenyl)-5-cyclopentyl-4,5-dihydro-1H-pyrazol-3-yl)-2-methoxynicotinic acid, Form A (Vertical Axis: Intensity (CPS); Horizontal Axis: Two theta (degrees))

FIG. 3 is a characteristic x-ray powder diffraction pattern showing a crystalline form of Example 4, (R)-6-(1-(4-cyano-3-methylphenyl)-5-cyclopentyl-4,5-dihydro-1H-pyrazol-3-yl)-2-methoxynicotinic acid, Form B (Vertical Axis: Intensity (CPS); Horizontal Axis: Two theta (degrees))

FIG. 4 is a characteristic x-ray powder diffraction pattern showing an amorphous form of Example 4, (R)-6-(1-(4-cyano-3-methylphenyl)-5-cyclopentyl-4,5-dihydro-1H-pyrazol-3-yl)-2-methoxynicotinic acid. (Vertical Axis: Intensity (CPS); Horizontal Axis: Two theta (degrees))

### DETAILED DESCRIPTION OF THE INVENTION

A preferred group of compounds, designated the A Group, contains those compounds having the Formula I as shown above wherein

### X is C or N;

### A is

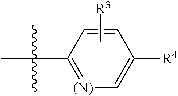

R¹ is halo, (C₁-C₆)alkyl or (C₁-C₄)alkoxy;
the pyrazoline C* is (R);
R² is cyclo(C₃-C₆)alkyl;
R³ is H, (C₁-C₄)alkylamino or (C₁-C₄)alkoxy; and
R⁴ is carboxy, carbamoyl, (C₁-C₄)alkylsulfonylaminocarbonyl or mono-N— or di-N—,N—(C₁-C₄)alkyaminocarbonyl.

A group of compounds which is preferred among the A Group of compounds, designated the B Group, contains those compounds wherein

### X IS C;

R¹ is in the position

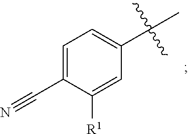

and
R³ is in the position

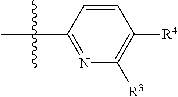

A group of compounds which is preferred among the B Group of compounds, designated the C Group, contains those compounds wherein

R¹ is halo or (C₁-C₄)alkyl;
R² is cyclopentyl;
R³ is (C₁-C₄)alkoxy; and
R⁴ is carboxy.

A group of compounds which is preferred among the B Group of compounds, designated the D Group, contains those compounds wherein

R¹ is halo or (C₁-C₄)alkyl;
R² is cyclopentyl;
R³ is (C₁-C₄)alkoxy; and
R⁴ is (C₁-C₄)alkylsulfonylaminocarbonyl

A group of compounds which is preferred among the B Group of compounds, designated the E Group, contains those compounds wherein

R¹ is halo or (C₁-C₄)alkyl;
R² is cyclopentyl;
R³ is (C₁-C₄)alkoxy; and
R⁴ is mono-N— or di-N—,N—(C₁-C₆)alkyaminocarbonyl.

A preferred group of compounds, designated the F Group, contains those compounds having the Formula I as shown above wherein

### X is N;

### A is

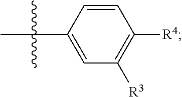

R¹ is halo, (C₁-C₆) alkyl or (C₁-C₆)alkoxy;
the pyrazoline C* is (R);
R² is cyclo(C₃-C₆)alkyl;
R³ is H, (C₁-C₄)alkylamino or (C₁-C₄)alkoxy; and
R⁴ is carboxy, carbamoyl, (C₁-C₄)alkylsulfonylaminocarbonyl or mono-N— or di-N—,N—(C₁-C₄)alkyaminocarbonyl.

A group of compounds which is preferred among the F Group of compounds, designated the G Group, contains those compounds wherein

R¹ is halo or (C₁-C₄)alkyl;
R² is cyclopentyl;
R³ is (C₁-C₄)alkoxy; and
R⁴ is carboxy, mono-N— or di-N—,N—(C₁-C₄)alkyaminocarbonyl
or (C₁-C₄)alkylsulfonylaminocarbonyl

A preferred group of compounds, designated the H Group, contains those compounds having the Formula I as shown above wherein

### X is N;

### A is

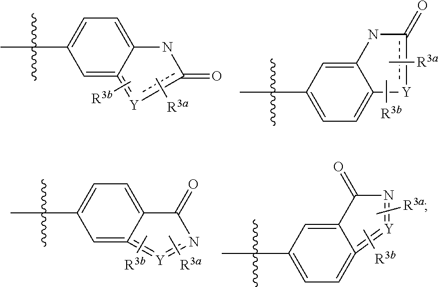

R^{3a} or R^{3b} is H or alkyl
R¹ is halo, (C₁-C₄) alkyl or (C₁-C₄)alkoxy;
the pyrazoline C* is (R); and
R² is cyclo(C₃-C₆)alkyl.

A preferred group of compounds, designated the I Group, contains those compounds having the Formula I as shown above wherein

### X is N;

### A is

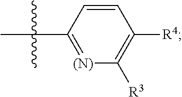

R¹ is halo, (C₁-C₄) alkyl or (C₁-C₄)alkoxy;
the pyrazoline C* is (R);
R² is cyclo(C₃-C₆)alkyl;
R³ is H, (C₁-C₄)alkylamino or (C₁-C₄)alkoxy; and
R⁴ is carboxy, carbamoyl, (C₁-C₄)alkylsulfonylaminocarbonyl or mono-N— or di-N—,N—(C₁-C₄)alkyaminocarbonyl.

A group of compounds which is preferred among the I Group of compounds, designated the J Group, contains those compounds wherein

R¹ is halo or (C₁-C₄)alkyl;
R² is cyclopentyl;
R³ is (C₁-C₄)alkoxy; and
R⁴ is carboxy, mono-N— or di-N—,N—(C₁-C₄)alkyaminocarbonyl.
or (C₁-C₄)alkylsulfonylaminocarbonyl

Especially preferred compounds having the Formula I are the compounds
(R)-6-(1-(4-cyano-3-methylphenyl)-5-cyclopentyl-4,5-dihydro-1H-pyrazol-3-yl)-2-methoxynicotinic acid;
(R)-4-(1-(5-cyano-6-methylpyridin-2-yl)-5-cyclopentyl-4,5-dihydro-1H-pyrazol-3-yl)-2-methoxybenzoic acid;
(R)-6-(1-(5-cyano-6-methylpyridin-2-yl)-5-cyclopentyl-4,5-dihydro-1H-pyrazol-3-yl)-2-methoxynicotinic acid;
(R)-4-(1-(5-cyano-6-methylpyridin-2-yl)-5-cyclopentyl-4,5-dihydro-1H-pyrazol-3-yl)-2-ethoxybenzoic acid;
(R)-6-(1-(4-cyano-3-methylphenyl)-5-cyclopentyl-4,5-dihydro-1H-pyrazol-3-yl)-2-ethoxynicotinic acid;
(R)-6-(1-(4-cyano-3-methylphenyl)-5-cyclopentyl-4,5-dihydro-1H-pyrazol-3-yl)-2-methoxy-N-(methylsulfonyl)nicotinamide; or
(R)-6-(1-(5-cyano-6-methylpyridin-2-yl)-5-cyclopentyl-4,5-dihydro-1H-pyrazol-3-yl)-2-methoxy-N-(methylsulfonyl)nicotinamide.

An especially preferred compound is 6-(1-(4-cyano-3-methylphenyl)-5-cyclopentyl-4,5-dihydro-1H-pyrazol-3-yl)-2-methoxynicotinic acid.

An especially preferred compound is (R)-6-(1-(4-cyano-3-methylphenyl)-5-cyclopentyl-4,5-dihydro-1H-pyrazol-3-yl)-2-methoxynicotinic acid or a pharmaceutically acceptable salt thereof.

An especially preferred compound is the compound of Formula II

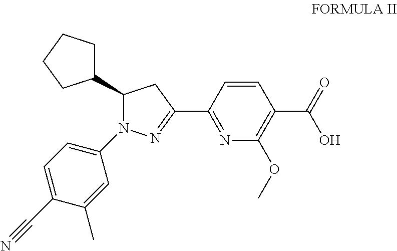

Pharmaceutically acceptable salts of the compounds of Formula I include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts Properties, Selection, and Use by Stahl and Wermuth (Wiley-VCH, 2002).

The compounds of the invention may exist in both unsolvated and solvated forms. The term ‘solvate’ is used herein to describe a molecular complex comprising the compound of the invention and one or more pharmaceutically acceptable solvent molecules, for example, ethanol. Such solvent molecules are those commonly used in the pharmaceutical art, which are known to be innocuous to the recipient, e.g., water, ethanol, ethylene glycol, and the like. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO. The term “hydrate” refers to the complex where the solvent molecule is water. The solvates and/or hydrates preferably exist in crystalline form.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of the invention include compounds of Formula I as hereinbefore defined, polymorphs, and isomers thereof (including optical, geometric and tautomeric isomers) as hereinafter defined and isotopically-labelled compounds of Formula I.

The compounds of the present invention may be administered as prodrugs. Thus certain derivatives of compounds of Formula I which may have little or no pharmacological activity themselves can, when administered into or onto the body, be converted into compounds of Formula I having the desired activity, for example, by hydrolytic cleavage. Such derivatives are referred to as ‘prodrugs’. [Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).]

Prodrugs can, for example, be produced by replacing appropriate functionalities present in the compounds of Formula I with certain moieties known to those skilled in the art as ‘pro-moieties’ as described, for example, in “Design of Prodrugs” by H Bundgaard (Elsevier, 1985).

Some examples of such prodrugs include:

i where the compound of Formula I contains a carboxylic acid functionality (—COOH), an ester thereof, for example, replacement of the hydrogen with (C₁-C₈)alkyl;
ii where the compound of Formula I contains an alcohol functionality (—OH), an ether thereof, for example, replacement of the hydrogen with (C₁-C₆)alkanoyloxymethyl; and
iii where the compound of Formula I contains a primary or secondary amino functionality (—NH₂ or —NHR where R≠H), an amide thereof, for example, replacement of one or both hydrogens with (C₁-C₁₀)alkanoyl.

In addition, certain compounds of Formula I may themselves act as prodrugs of other compounds of Formula I.

Compounds of Formula I containing an asymmetric carbon atom can exist as two or more stereoisomers. Where a compound of Formula I contains an alkenyl or alkenylene group or a cycloalkyl group, geometric cis/trans (or Z/E) isomers are possible. Where the compound contains, for example, a keto or oxime group or an aromatic moiety, tautomeric isomerism ('tautomerism') can occur. It follows that a single compound may exhibit more than one type of isomerism.

Included within the scope of the claimed compounds present invention are all stereoisomers, geometric isomers and tautomeric forms of the compounds of Formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

The present invention includes all pharmaceutically acceptable isotopically-labelled compounds of Formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of Formula (I) can generally be prepared by conventional techniques known to those skilled in the art or by processes analogous to those described in the accompanying Examples and Preparations using an appropriate isotopically-labelled reagents in place of the non-labelled reagent previously employed.

References herein to “treatment” include curative, palliative and prophylactic treatment.

As used herein, the expressions “reaction-inert solvent” and “inert solvent” refer to a solvent or a mixture thereof which does not interact with starting materials, reagents, intermediates or products in a manner which adversely affects the yield of the desired product.

By “pharmaceutically acceptable” is meant the carrier, diluent, excipients, and/or salt must be compatible with the other ingredients of the Formulation, and not deleterious to the recipient thereof.

The term “pharmaceutically effective amount”, as used herein, refers to an amount of the compound of Formula I sufficient to treat, prevent onset of or delay or diminish the symptoms and physiological manifestations of the indications described herein.

The term “room temperature” means a temperature between 18 to 25° C., “HPLC” refers to high pressure liquid chromatography, “MPLC” refers to medium pressure liquid chromatography, “TLC” refers to thin layer chromatography, “MS” refers to mass spectrum, “NMR” refers to nuclear magnetic resonance spectroscopy, “DCM” refers to dichloromethane, “DMSO” refers to dimethyl sulfoxide, “DME” refers to dimethoxyethane, “EtOAc” refers to ethyl acetate, “MeOH” refers to methanol, “Ph” refers to the phenyl group, “Pr” refers to propyl, “trityl” refers to the triphenylmethyl group, “ACN” refers to acetonitrile, “DEAD” refers to diethylazodicarboxylate, and “DIAD” refers to diisopropylazodicarboxylate.

The phrase “wherein at least X is N or the A substituent contains a ring nitrogen” (underline added for emphasis) also includes both X being N and the A substituent containing a ring nitrogen.

Alkyl, alkenyl and alkynyl groups and the alkyl portions of alkoxy groups discussed herein include straight or branched groups having the number of carbon atoms indicated including, for example, methyl, methoxy, ethyl, styrene, propyl, isopropyl, isopropyloxy, allyl, n-butyl, t-butyl, isobutyl, pentyl, isopentyl, and 2-methylbutyl groups. The terms halo or halogen refer to F, Cl, Br or I.

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth.

In general the compounds of this invention can be made by processes which include processes analogous to those known in the chemical arts, particularly in light of the description contained herein. Certain processes for the manufacture of the compounds of this invention are provided as further features of the invention and are illustrated by the following reaction schemes. Other processes may be described in the experimental section.

Specific synthetic schemes for preparation of the compounds of Formula I are outlined below.

As an initial note, in the preparation of the Formula I compounds it is noted that some of the preparation methods useful for the preparation of the compounds described herein may require protection of remote functionality (e.g., primary amine, secondary amine, carboxyl in Formula I precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the Formula I compound.

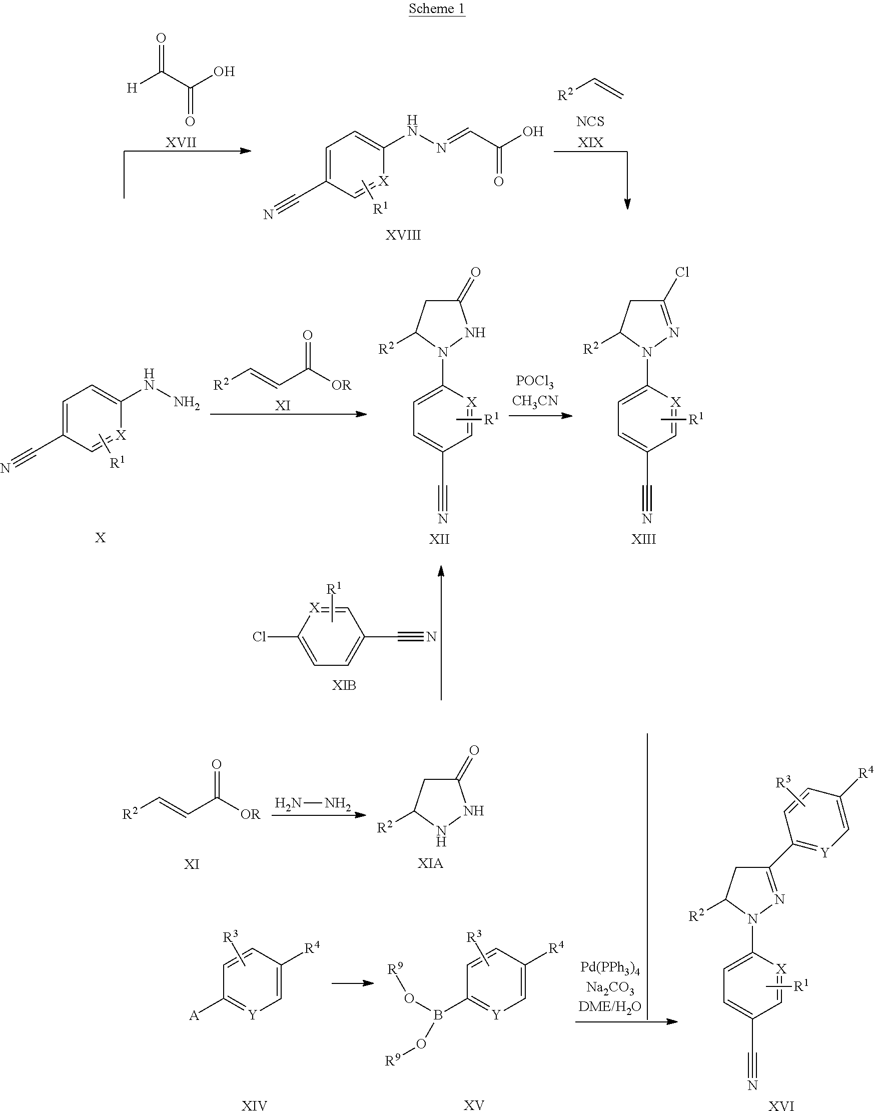

According to Scheme 1 the Formula XVI compounds wherein X, R¹, R², R³ and R⁴ are as defined above and Y is C or N may be prepared from the Formula X compound by cyclization, subsequent conversion to the chloride, and a Suzuki coupling with an appropriate Formula XV compound (wherein R³ and R⁴ are as defined above and Y is CH or N).

Thus, the Formula XII compounds wherein R¹ and R² are as defined above may be prepared from the appropriate Formula X and Formula XI compounds, wherein R is typically an alkyl group e.g., methyl or ethyl and R¹ and R² are appropriate to achieve the desired Formula XII compounds by cyclization.

For example, the Formula XI compound may be conveniently prepared by combining sodium ethoxide and triethyl phosphonoacetate in a polar aprotic solvent such as methyltetrahydrofuran at a temperature of about −20° C. to about 20° C., typically less than 0° C., for about 10 minutes to about two hours. Then a R²carboxaldehyde (e.g., cyclopentancarboxaldehyde), appropriate to achieve the desired Formula XI compound, is added over about 30 minutes to about three hours, followed by warming to ambient temperature over about ten to about twenty hours to prepare the desired Formula XI compound.

The Formula XII compound may be prepared by combining the resulting Formula XI compound and the appropriate Formula X compound in an aprotic solvent such as tetrahydrofuran in the presence of a strong base such as potassium t-butoxide at a temperature of about 25° C. to about 100° C., typically about reflux for about 1 hour to about six hours.

The Formula XII compound is converted to the chloride derivative to achieve the desired Formula XIII compound with phosphorous oxychloride in a polar solvent such as acetonitrile at a temperature of about 25° C. to about 100° C., typically about 80° C. under an inert atmosphere for about 2 to about 24 hours.

The desired Formula XV compound wherein R³ and R⁴ are as defined above, Y is CH or N, and R⁹ is either H or alkyl, or taken together with the other R⁹ group to form a heterocycloalkyl derivative e.g., pinacolate derivative, is prepared from the appropriate Formula XIV compound wherein R³ and R⁴ are as defined above, Y is CH or N and A is bromo or chloro by palladium-catalyzed boronylation, or metalation/boronylation followed by acid hydrolysis.

For example, the Formula XIV compound is treated with a mixture of a catalyst such as [1,1-bis(diphenylphosphino)ferrocene]palladium (II) chloride, a base such as potassium acetate and a borylation reagent such as bis(pinacolate)diborane in a polar, aprotic solvent such as dichloromethane. The compounds are combined at an elevated temperature of about 40° C. to about 120° C., approximately 80° C. under an inert atmosphere for about two to about twelve hours to achieve the desired Formula XV compound.

The desired Formula XVI compound is prepared by Suzuki coupling of the appropriate Formula XV compound and Formula XIII compound.

For example, the Formula XV compound and Formula XIII compound are coupled with palladium tetrakis(triphenylphosphine) in an aprotic solvent such as dimethoxyethane (DME) in the presence of an excess of sodium carbonate at elevated temperatures of about 80° C. to about 100° C., typically reflux under an inert atmosphere for about two to about twelve hours.

In addition, the desired Formula XVI compound, wherein the mono-cyclic ring having the R³ and R⁴ substituents is instead a bicyclic moiety (i.e., the bicyclic A moiety described herein above), may be prepared in an analogous manner to that described immediately above or below.

Alternatively the Formula XIII compound may be prepared by combining the appropriate Formula XVIII compound wherein R¹ is as defined above and Formula XIX compound wherein R² is as defined above.

For example, the Formula XVIII compound is combined with the appropriate Formula XIX vinyl compound and N-chlorosuccinimide in a solvent such as ethyl acetate in the presence of a base such as sodium bicarbonate at ambient temperatures of about 15° C. to about 35° C., under an inert atmosphere for about ten hours to about two days followed by heating at elevated temperatures of about 50° C. to about 100° C. for about three hours to about twelve hours.

The Formula XVIII compound wherein R¹ is as defined above may be prepared by combining glyoxylic acid and the appropriate substituted hydrazine compound X in an polar solvent such as water at ambient temperatures of about 15° C. to about 35° C., under an inert atmosphere for about ten hours to about two days.

Alternatively, and in particular wherein X is N the Formula XII compound may be prepared by aromatic nucleophilic substitution reaction of the appropriate Formula XIA with XIB compounds.

For example, the Formula XIA compound and Formula XIB compound are combined in a polar solvent such as water and heated to a temperature of about 125° C. to about 175° C., under an inert atmosphere for about 10 minutes to about one hour.

The Formula XIA compound wherein R² is as defined above may be prepared by combining the appropriate Formula XI compound wherein R² is as defined above with hydrazine hydrate in a polar solvent such as ethanol at ambient temperatures of about 15° C. to about 35° C., under an inert atmosphere for about 30 minutes to about two hours followed by elevated temperatures of about 70° C. to about 100° C., typically reflux under an inert atmosphere for about twelve hours to about 48 hours.

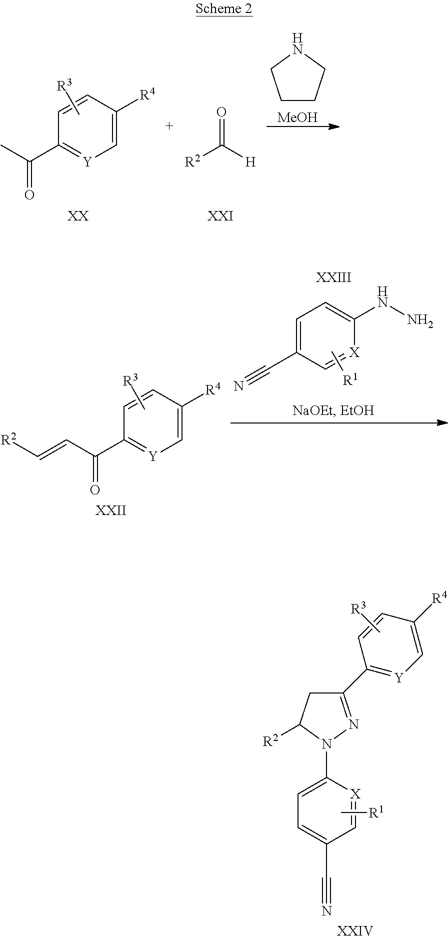

According to Scheme 2 the Formula XXIV compounds wherein X, R¹, R², R³ and R⁴ are as defined above and Y is C or N may be prepared by an aldol reaction to form an alpha, beta unsaturated ketone and subsequent cyclization with a substituted hydrazine derivative.

Thus, the Formula XXII compound wherein R², R³ and R⁴ are as defined above and Y is C or N may be prepared from the appropriate Formula XX and Formula XXI aldehyde by an aldol reaction. For example, the Formula XX compound is combined with the Formula XXI aldehyde in a protic solvent such as methanol and an amine base such as pyrrolidine is added at a temperature of about −20° C. to about 20° C., typically about 0° C. under an inert atmosphere for about one minute to about three hours. The reaction is allowed to warm to ambient temperature and stirred for about ten minutes to about six hours.

The resulting Formula XXII compound is coupled with a Formula XXIII compound in a protic solvent such as ethanol in the presence of a strong base (e.g., metal alkoxide such as sodium ethoxide) at a temperature of about 40° C. to about 120° C., typically about 80° C. under an inert atmosphere for about one hour to about six hours. The reaction is allowed to cool to ambient temperature resulting in the desired Formula XXIV compound pyrazoline.

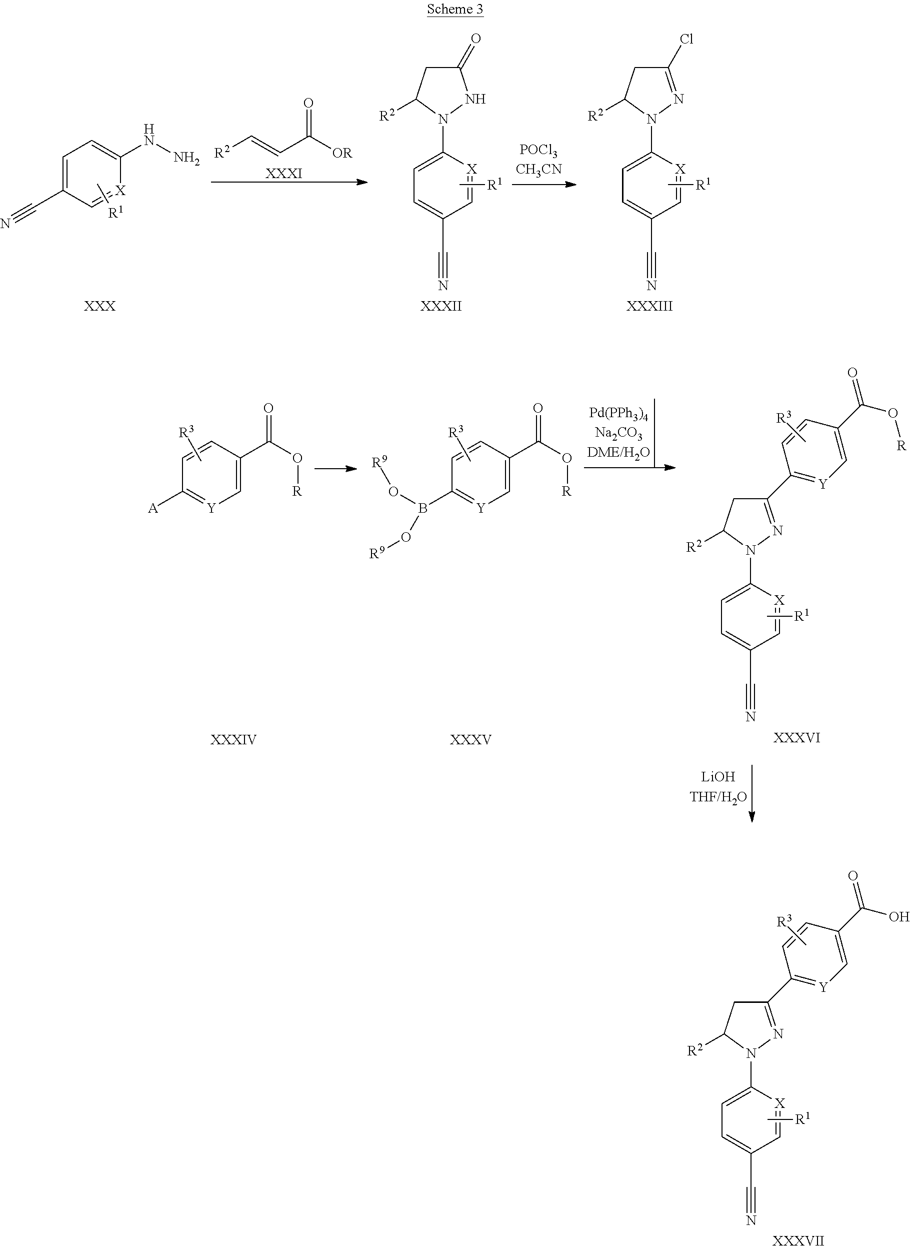

According to Scheme 3 the Formula XXXVII compounds wherein X, R¹, R², and R³ are as defined above, R⁴ is 4-carboxy and Y is C or N may be prepared by cyclization, subsequent conversion to the chloride followed by Suzuki coupling and hydrolysis. Thus, the Formula XXXII compounds wherein R¹ and R² are as defined above may be prepared from the appropriate Formula XXX and Formula XXXI compounds wherein R is typically an alkyl group e.g., methyl or ethyl and R¹ and R² are appropriate to achieve the desired Formula XII compounds by cyclization.

For example, the Formula XXXI compound may be conveniently prepared by combining sodium ethoxide and triethyl phosphonoacetate in a polar aprotic solvent such as methyltetrahydrofuran at a temperature of about −20° C. to about 20° C., typically less than 0° C., for about 10 minutes to about two hours. Then a R²carboxaldehyde (e.g., cyclopentancarboxaldehyde), appropriate to achieve the desired Formula XXXI compound, is added over about 30 minutes to about three hours, followed by warming to ambient temperature over about ten to about twenty hours to prepare the desired Formula XXXI compound.

The Formula XXXII compound may be prepared by combining the resulting Formula XXXI compound and the appropriate Formula XXX compound in an aprotic solvent such as tetrahydrofuran in the presence of a strong base such as potassium t-butoxide at a temperature of about 25° C. to about 100° C., typically about reflux for about 1 hour to about six hours.

The Formula XXXII compound is converted to the chloride derivative to achieve the desired Formula XXXIII compound with phosphorous oxychloride in a polar solvent such as acetonitrile at a temperature of about 25° C. to about 100° C., typically about 80° C. under an inert atmosphere for about 2 to about 24 hours.

The desired Formula XXXV compound wherein R³ and R⁴ are as defined above, Y is CH or N, R⁹ is either H or alkyl, or taken together with the other R⁹ group to form a heterocycloalkyl derivative e.g., pinacolate derivative, is prepared from the appropriate Formula XXXIV compound wherein R³ and R⁴ are as defined above, Y is CH or N and A is bromo or chloro by palladium-catalyzed boronylation, or metalation/boronylation followed by acid hydrolysis.

For example, the Formula XXXIV compound is treated with a mixture of a catalyst such as [1,1-bis(diphenylphosphino)ferrocene]palladium (II) chloride, a base such as potassium acetate and a borylation reagent such as bis(pinacolate)diborane in a polar, aprotic solvent such as dichloromethane. The compounds are combined at an elevated temperature of about 40° C. to about 120° C., approximately 80° C. under an inert atmosphere for about two to about twelve hours to achieve the desired Formula XXXV compound.

The Formula XXXVI ester is prepared by Suzuki coupling of the appropriate Formula XXXIII compound and Formula XXXV compound. For example, the Formula) XXXIII compound and Formula XXXV compound are coupled with palladium tetrakis(triphenylphosphine) in an aprotic solvent such as dimethoxyethane (DME), toluene or DMF in the presence of an excess of sodium carbonate at elevated temperatures of about 80° C. to about 100° C., typically reflux under an inert atmosphere for about two to about twelve hours.

The resulting Formula XXXVI ester can be simply hydrolyzed to the corresponding Formula XXXVII acid. For example, the ester is dissolved into a aprotic solvent such as tetrahydrofuran and a strong base such as lithium hydroxide is added followed by heating at elevated temperatures of about 30° C. to about 60° C., typically about 40° C. under an inert atmosphere for about two to about twelve hours.

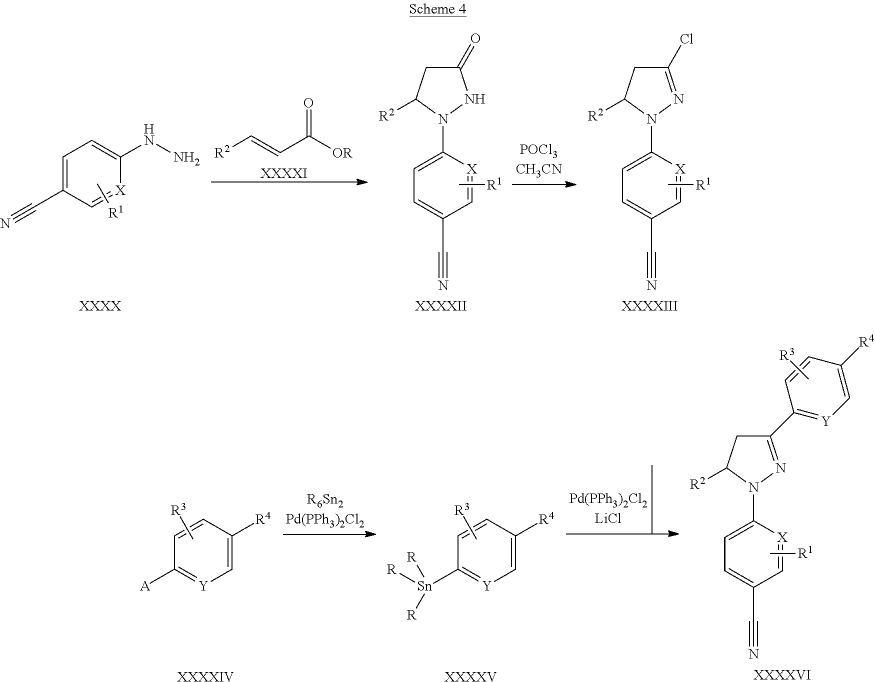

According to Scheme 4 the Formula XXXXVI compounds wherein X, R¹, R², R³, and R⁴ are as defined above and Y is C or N may be prepared from the Formula XXXX compound by cyclization, subsequent conversion to the chloride and a Stille coupling.

Thus, the Formula XXXXII compounds wherein R¹ and R² are as defined above may be prepared from the appropriate Formula XXXX and Formula XXXXI compounds wherein R is typically an alkyl group e.g., methyl or ethyl and R¹ and R² are appropriate to achieve the desired Formula XXXII compounds by cyclization.

For example, the Formula XXXXI compound may be conveniently prepared by combining sodium ethoxide and triethyl phosphonoacetate in a polar aprotic solvent such as methyltetrahydrofuran at a temperature of about −20° C. to about 20° C., typically less than 0° C., for about 10 minutes to about two hours. Then a R²carboxaldehyde (e.g., cyclopentancarboxaldehyde), appropriate to achieve the desired Formula XXXXI compound, is added over about 30 minutes to about three hours, followed by warming to ambient temperature over about ten to about twenty hours to prepare the desired Formula XXXXI compound.

The Formula XXXXII compound may be prepared by combining the resulting Formula XXXXI compound and the appropriate Formula XXXX compound in an aprotic solvent such as tetrahydrofuran in the presence of a strong base such as potassium t-butoxide at a temperature of about 25° C. to about 100° C., typically about reflux for about 1 hour to about six hours.

The Formula XXXXII compound is converted to the chloride derivative to achieve the desired Formula XXXXIII compound with phosphorous oxychloride in a polar solvent such as acetonitrile at a temperature of about 25° C. to about 100° C., typically about 80° C. under an inert atmosphere for about 2 to about 24 hours.

The desired Formula XXXXV compound wherein R³ and R⁴ are as defined above, Y is CH or N and SnR₃ is a trialkyl group, typically a tributyl group is prepared from the appropriate Formula XXXXIV compound wherein R³ and R⁴ are as defined above, Y is CH or N and A is bromo or chloro by palladium-catalyzed stannylation.

For example, the Formula XXXIV compound is treated with a mixture of an organotin reagent such as bis(tributyltin) and a catalyst such as bis(triphenylphosphine)palladium (II) chloride in an aprotic solvent such as anhydrous dioxane at an elevated temperature of about 60° C. to about 140° C., approximately 100° C. under an inert atmosphere e.g., argon for about two to about twelve hours. The reaction is heated until complete as needed.

The Formula XXXXVI ester is prepared by a Stille coupling of the appropriate Formula XXXIII compound and Formula XXXXV compound. For example, the Formula XXXIII compound and Formula XXXXV compound are coupled with bis(triphenylphosphine)palladium (II) chloride and lithium chloride in an aprotic solvent such as dimethoxyethane (DME), toluene or DMF at elevated temperatures of about 60° C. to about 140° C., typically about 100° C. under an inert atmosphere for about two to about twelve hours.

The starting materials and reagents for the above described Formula I compounds, are also readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

Cis/trans isomers may be separated by conventional techniques well known to those skilled in the art, for example, chromatography and fractional crystallization.

Mixtures of stereoisomers may be separated by conventional techniques known to those skilled in the art. [see, for example, “Stereochemistry of Organic Compounds” by E L Eliel (Wiley, New York, 1994).]

Conventional techniques for the preparation/isolation of individual enantiomers include chiral synthesis from a suitable optically pure precursor.

Alternatively, the racemate (or a racemic precursor) may be reacted with a suitable optically active compound, for example, an alcohol, or, in the case where the compound of Formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Pharmaceutically acceptable salts of compounds of Formula I may be prepared by one or more of three methods:
(i) by reacting the compound of Formula I with the desired acid or base;
(ii) by removing an acid- or base-labile protecting group from a suitable precursor of the compound of Formula I or by ring-opening a suitable cyclic precursor, for example, a lactone or lactam, using the desired acid or base; or
(iii) by converting one salt of the compound of Formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionized to almost non-ionized.

The compounds of this invention may also be used in conjunction with other pharmaceutical agents (e.g., antihypertensive and antidiabetic agents) for the treatment of the disease/conditions described herein.

The compounds of the present invention may be used in combination with antihypertensive agents and such antihypertensive activity is readily determined by those skilled in the art according to standard assays (e.g., blood pressure measurements). Exemplary antihypertensive agents include rennin inhibitors (e.g., aliskiren), aldosterone synthase inhibitors, calcium channel blockers, angiotensin converting enzyme inhibitors (ACE inhibitors), angiotensin II receptor antagonists (ARB antagonists), Beta-adrenergic receptor blockers (beta- or β-blockers), Alpha-adrenergic receptor blockers (alpha- or α-blockers), vasodilators such as cerebral vasodilators, coronary vasodilators, peripheral vasodilators and diuretics.

In one embodiment, one or more compounds of Formulae I or II may be co-administered with one or more diuretics. Examples of suitable diuretics include (a) loop diuretics such as furosemide (such as LASIX™), torsemide (such as DEMADEX™), bemetanide (such as BUMEX™), and ethacrynic acid (such as EDECRIN™); (b) thiazide-type diuretics such as chlorothiazide (such as DIURIL™, ESIDRIX™ or HYDRODIURIL™), hydrochlorothiazide (such as MICROZIDE™ or ORETIC™), benzthiazide, hydroflumethiazide (such as SALURON™), bendroflumethiazide, methychlorthiazide, polythiazide, trichlormethiazide, and indapamide (such as LOZOL™); (c) phthalimidine-type diuretics such as chlorthalidone (such as HYGROTON™), and metolazone (such as ZAROXOLYN™); (d) quinazoline-type diuretics such as quinethazone; and (e) potassium-sparing diuretics such as triamterene (such as DYRENIUM™), and amiloride (such as MIDAMOR™ or MODURETIC™).

In another embodiment, one or more compounds of Formulae I or II may be co-administered with a loop diuretic. In still another embodiment, the loop diuretic is selected from furosemide and torsemide. In still another embodiment, one or more compounds of Formulae I or II may be co-administered with furosemide. In still another embodiment, one or more compounds of Formulae I or II may be co-administered with torsemide which may optionally be a controlled release form of torsemide.

In another embodiment, one or more compounds of Formulae I or II may be co-administered with a thiazide-type diuretic. In still another embodiment, the thiazide-type diuretic is selected from the group consisting of chlorothiazide and hydrochlorothiazide. In still another embodiment, one or more compounds of Formulae I or II may be co-administered with chlorothiazide. In still another embodiment, one or more compounds of Formulae I or II may be co-administered with hydrochlorothiazide.

In another embodiment, one or more compounds of Formulae I or II may be co-administered with a phthalimidine-type diuretic. In still another embodiment, the phthalimidine-type diuretic is chlorthalidone.

The compounds of the present invention may be used in combination with antidiabetic agents and such anti-diabetic activity is readily determined by those skilled in the art according to standard assays known in the art. Examples of such antidiabetic agents include an acetyl-CoA carboxylase-2 (ACC-2) inhibitor, a phosphodiesterase (PDE)-10 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., exendin-3 and exendin-4, exenatide (Byetta□)), a protein tyrosine phosphatase-1B (PTP-1B) inhibitor (e.g., trodusquemine, hyrtiosal extract, and compounds disclosed by Zhang, S., et al., Drug Discovery Today, 12 (9/10), 373-381 (2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist, 11 Beta HSD and a glucokinase activator. Preferred anti-diabetic agents are metformin, glucagon-like peptide 1 (GLP-1) agonists (Byetta), and DPP-IV inhibitors (e.g., sitagliptin, vildagliptin, alogliptin and saxagliptin).

The compounds of the present invention may be used in combination with cholesterol modulating agents (including cholesterol lowering agents) such as a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, an ion-exchange resin, an antioxidant, an ACAT inhibitor or a bile acid sequestrant.

The compounds of the present invention can be used in combination with anti-obesity agents. Such anti-obesity activity is readily determined by those skilled in the art according to standard assays known in the art. Suitable anti-obesity agents include phenylpropanolamine, ephedrine, pseudoephedrine, phentermine, β₃ adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine), sympathomimetic agents, serotoninergic agents, cannabinoid receptor (CB-1) antagonists (e.g., rimonabant described in U.S. Pat. No. 5,624,941 (SR-141,716A), purine compounds, such as those described in US Patent Publication No. 2004/0092520; pyrazolo[1,5-a][1,3,5]triazine compounds, such as those described in U.S. Non-Provisional patent application Ser. No. 10/763,105 filed on Jan. 21, 2004; and bicyclic pyrazolyl and imidazolyl compounds, such as those described in U.S. Provisional Application No. 60/518,280 filed on Nov. 7, 2003), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-Y antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine™), human agouti-related proteins (AGRP), ghrelin receptor antagonists, histamine 3 receptor antagonists or inverse agonists, neuromedin U receptor agonists, and the like.

The compounds of this invention may also be used in combination with a lipase inhibitor. A lipase inhibitor is a compound that inhibits the metabolic cleavage of dietary triglycerides or plasma phospholipids into free fatty acids and the corresponding glycerides (e.g. EL, HL, etc.). Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a glyceride and fatty acid. In the intestine, the resultant free fatty acids and monoglycerides are incorporated into bile acid-phospholipid micelles, which are subsequently absorbed at the level of the brush border of the small intestine. The micelles eventually enter the peripheral circulation as chylomicrons. Such lipase inhibition activity is readily determined by those skilled in the art according to standard assays (e.g., Methods Enzymol. 286: 190-231).

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology, 92,125 (1987). Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art.

In combination therapy treatment, both the compounds of this invention and the other drug therapies are administered to mammals (e.g., humans, male or female) by conventional methods.

The Formula I compounds of this invention, their prodrugs and the salts of such compounds and prodrugs are all adapted to therapeutic use as agents that mediate the mineralocorticoid receptor (MR) in mammals, particularly humans. For example, these compounds act as mineralocorticoid receptor antagonists (MRa) and thus are useful for the treatment of the various conditions (e.g., those described herein) in which such action is implicated.

It is believed that the mineralocorticoids, such as aldosterone, are involved in regulating salt and water balance in mammals. Activation of the mineralocorticoid receptor can induce hypertension and cause other detrimental cardiovascular and physiological effects. Accordingly, MR antagonists help to reduce hypertension and associated physiological effects.

Given the positive correlation between activation of the mineralocorticoid receptor with the development of cardiovascular and associated disease/conditions, Formula I compounds of this invention, their prodrugs and the salts of such compounds and prodrugs, by virtue of their pharmacologic action, are useful for the prevention, arrestment and/or regression of hypertension and its associated disease states. These include cardiovascular disorders (e.g., angina, cardiac ischemia and myocardial infarction) and other associated complications e.g., diabetic nephropathy.

The disease/conditions that can be treated in accordance with the present invention include, but are not limited to, cardiovascular conditions, renal conditions, liver conditions, vascular conditions, inflammatory conditions, pain, retinopathy, neuropathy (such as peripheral neuropathy), insulinopathy, edema, endothelial dysfunction, baroreceptor dysfunction and the like.

Cardiovascular conditions include, but are not limited to, hypertension, heart failure (such as congestive heart failure), diastolic dysfunction (such as left ventricular diastolic dysfunction, diastolic heart failure, and impaired diastolic filling), systolic dysfunction (such as systolic heart failure), arrhythmia, ischemia, hypertrophic cardiomyopathy, sudden cardiac death, myocardial and vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, fibrinoid necrosis of coronary arteries, stroke, and the like.

Renal conditions include, but are not limited to, glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, macroalbuminuria, renal arteriopathy, ischemic lesions, thrombotic lesions, global fibrinoid necrosis, focal thrombosis of glomerular capillaries, swelling and proliferation of intracapillary (endothelial and mesangial) and/or extracapillary cells (crescents), expansion of reticulated mesangial matrix with or without significant hypercellularity, malignant nephrosclerosis (such as ischemic retraction, thrombonecrosis of capillary tufts, arteriolar fibrinoid necrosis, and thrombotic microangiopathic lesions affecting glomeruli and microvessels), and the like.

Liver conditions include, but are not limited to, liver cirrhosis, liver ascites, hepatic congestion, and the like.

Vascular conditions include, but are not limited to, thrombotic vascular disease (such as mural fibrinoid necrosis, extravasation and fragmentation of red blood cells, and luminal and/or mural thrombosis), proliferative arteriopathy (such as swollen myointimal cells surrounded by mucinous extracellular matrix and nodular thickening), atherosclerosis, decreased vascular compliance (such as stiffness, reduced ventricular compliance and reduced vascular compliance), endothelial dysfunction, and the like.

Inflammatory conditions include, but are not limited to, arthritis (for example, osteoarthritis), inflammatory airways diseases (for example, chronic obstructive pulmonary disease (COPD)), and the like.

Pain includes, but is not limited to, acute pain, chronic pain (for example, arthralgia), and the like.

Edema includes, but is not limited to, peripheral tissue edema, hepatic congestion, splenic congestion, liver ascites, respiratory or lung congestion, and the like.

Insulinopathies include, but are not limited to, insulin resistance, Type I diabetes mellitus, Type II diabetes mellitus, glucose sensitivity, pre-diabetic state, syndrome X, and the like.

In one embodiment, the condition is selected from the group consisting of cardiovascular conditions, renal conditions, and liver conditions.

In another embodiment, the condition is a cardiovascular condition.

In another embodiment, the condition is a cardiovascular condition selected from the group consisting of hypertension, heart failure (particularly heart failure post myocardial infarction), left ventricular hypertrophy, and stroke.

In another embodiment, the condition is hypertension.

In another embodiment, the condition is heart failure.

In another embodiment, the condition is left ventricular hypertrophy.

In another embodiment, the condition is stroke.

In another embodiment, the condition is a renal condition.

In another embodiment, the condition is diabetic nephropathy.

In another embodiment, the condition is Type II diabetes mellitus.

The compounds of Formula I can have improved solubility and selectivity across related nuclear hormone receptors including progesterone, androgen and glucocorticoid.

The utility of the Formula I compounds of the invention, their prodrugs and the salts of such compounds and prodrugs as medical agents in the treatment of the above described disease/conditions in mammals (e.g. humans, male or female) is demonstrated by the activity of the compounds of this invention in conventional in vitro and in vivo assays described below. The in vivo assays (with appropriate modifications within the skill in the art) may be used to determine the activity of other agents as well as the compounds of this invention. Such assays also provide a means whereby the activities of the Formula I compounds of this invention, their prodrugs and the salts of such compounds and prodrugs (or the other agents described herein) can be compared to each other and with the activities of other known compounds. The results of these comparisons are useful for determining dosage levels in mammals, including humans, for the treatment of such diseases.

The following protocols may of course be varied by those skilled in the art.

### Radioligand Binding Assay

To measure the affinity of test compound in the present invention for MR, and therefore have the capacity to modulate MR activity, radioligand displacement assays were performed. Test compound affinity was expressed as IC₅₀ value, defined as the concentration of test compound required to decrease [³H]aldosterone binding by 50%.

MR binding assays were performed in a final volume of 50 μL containing 1 nM of MR (GST-LBD fusion; expressed in SF9 insect cells), and 1 nM [³H]aldosterone (PerkinElmer, NET419) plus varying concentrations of test compound or vehicle.

Briefly, assays were prepared at 4° C. in 384-well plate (Costar, 3657) containing 1 μl of test compound in DMSO (or DMSO as vehicle). Assays were initiated by addition of 24 μL of 2 nM [³H]aldosterone followed by 25 μl_of 2 nM GST-MR in binding-wash buffer (50 mM HEPES (pH 7.5), 50 mM KCl, 2 mM EDTA, 10% glycerol and 5 mM DTT).

The mixture was incubated at 4° C. for 4 hrs, then was transferred to a 384-well glass fiber filtration plate (Millipore, MZFCN0W50) previously treated with 0.5% PEI. The mixture was suctioned dry with vacuum and immediately washed three times with 100 μL of 4° C. binding-wash buffer. The plates were allowed to air dry overnight at room temperature, 7 μL of Ready Safe Liquid Scintillant (Beckman, 141349) was added to each well, and the amount of receptor-ligand complex was determined by liquid scintillation counting using a 1450 Microbeta Trilux (Wallac).

Radioligand binding filtration format assays for progesterone receptor (PR) were performed in an identical manner as described for MR except 4 nM (final concentration) full length PR (Invitrogen, P2835) was substituted for MR and 1 nM (final concentration) [³H]progesterone (PerkinElmer, NET381) was substituted for radiolabeled aldosterone.

### Cell-Based Reporter Assay

To measure the ability of test compound in the present invention to modulate the activity of MR (agonize, antagonize, partially agonize, partially antagonize), bioassays were performed that which measured the modulation of target gene expression in cells transiently transfected with a plasmid containing the Gal4 DNA binding domain (DBD) fused to the LBD of MR and a plasmid containing the response element of Gal4 driving the luciferase reporter gene. An agonist of the receptor can bind to and activate the receptor LBD GAI4 DBD fusion, leading to activation of the luciferase reporter gene. An antagonist can compete for binding to the receptor LBD and decrease the transcriptional activity of the reporter gene. Measurement of luciferase activity allows quantitative determinations of the reporter transcription in the presence of either agonists alone or agonists and antagonists in combination.

Briefly, human liver cells (Huh7) were transfected using FuGENE™ 6 Transfection Reagent according to the manufacturer's instructions (Roche Molecular Biochemicals, 11814443001). Approximately 24 hours after transfection, the cells were harvested in phenol red-free RPMI1640 media containing 10% charcoal-and-dextran stripped serum (HyClone, SH30068.03), and plated in 45 μl at 7,500 cells per well in a CulturPlate™ 384-microplate (Perkin Elmer, 6007688). To test for receptor antagonism, cells were incubated for approximately 2 hours and treated with 5 μL of agonist aldosterone at EC₈₀ (concentration required for 80% of full activation for MR) plus test compound. For preparation of test sample, test compound was dissolved in DMSO, further diluted to various stock concentrations in DMSO and to ten-fold final concentrations in phenol red-free media plus 10% charcoal-and-dextran stripped serum containing aldosterone at ten-fold EC₈₀. The final concentration of DMSO in the test plate was 0.25%. Following an overnight incubation with compound, 25 μL of Steady-Glow™ lysis buffer with luciferase substrate (Promega Corporation, E2550) was added directly to the cells. After a 30-minute incubation to completely lyse the cells, the microplates were counted in an Envision™ Multilabel Reader (Perkin Elmer) in single photon counting mode. In antagonist mode, compound efficacy was expressed as IC₅₀ value, defined as the concentration of test compound required to decrease the EC₈₀ aldosterone signal by 50%. Examples 3, 6, 12 and 13 were tested in an analogous manner (minor variations) to the format described above. (See TABLE 1)

A cell-based reporter assay measuring the ability of test compound to modulate the activity of PR was performed in an identical manner as described for MR except cells were transfected with plasmid encoding the DNA binding domain of Gal4 fused to the LBD of PR rather than MR, and progesterone was used as agonist.

Glucocorticoid and androgen receptor assays were performed in a similar manner to MR, except the appropriate Gal4-LBDs were used, the assays were performed in 96-well density (Corning, 3596) by adding 30,000 cells to each well in a volume of 100 μL, test compound and agonist (dexamethasone and dihydrotestosterone, respectively) were added in a 3-fold concentrated stock in 50 μL volume, and Steady-Glow™ lysis buffer was added in 50 μL volume.

### Abbreviations

HEPES 4-(2-hydroxyethyl)-1-piperazineethanesulfonic acid
DTT dithiothreitol
EDTA ethylenediaminetetraacetic acid
GST glutathione S-transferase
LBD ligand binding domain
PEI polyethylene imine

| Example | MR IC50 (μM) |
|---|---|
| 1 | 0.0346 |
| 2 | 0.0092 |
| 3 | 0.011 |
| 4 | 0.0042 |
| 5 | 4.45 |
| 6 | 0.009 |
| 7 | 0.0179 |
| 8 | 0.0043 |
| 9 | 1.310 |
| 10 | 0.235 |
| 11 | 0.018 |
| 12 | 0.01 |
| 13 | 2 |
| 14 | >2.26 |
| 15 | 0.0214 |
| 16 | 0.0134 |
| 17 | 0.0084 |
| 18 | 0.0019 |
| 19 | 0.0108 |
| 20 | 0.0197 |
| 21 | 0.105 |
| 22 | 0.0135 |
| 23 | 0.113 |
| 24 | 0.0250 |
| 25 | 0.0484 |
| 26 | 0.0853 |
| 27 | 0.0052 |
| 28 | 0.0976 |
| 29 | 0.0306 |
| 30 | 0.0097 |
| 31 | 0.0112 |
| 32 | Not tested |
| 33 | 0.0076 |
| 34 | 0.264 |
| 35 | 0.0089 |
| 36 | 0.225 |
| 37 | Not tested |
| 38 | 0.0822 |
| 39 | 0.0070 |
| 40 | 0.0027 |
| 41 | 0.0091 |
| 42 | 0.005 |
| 43 | 0.13 |
| 44 | 0.0024 |
| 45 | 0.0206 |
| 46 | 0.042 |
| 47 | 0.012 |
| 48 | Not tested |
| 49 | 0.0028 |
| 50 | Not tested |
| 51 | 0.0082 |
| 52 | 0.0623 |
| 53 | Not tested |
| 54 | 0.0957 |
| 55 | 0.0039 |
| 56 | 0.0178 |
| 57 | 0.503 |
| 58 | 0.0033 |
| 59 | 0.0037 |
| 60 | 0.0094 |
| 61 | 0.208 |
| 62 | 0.0923 |
| 63 | 0.0022 |
| 64 | 0.01 |
| 65 | 0.0024 |
| 66 | 0.255 |
| 67 | 0.17 |
| 68 | 0.0041 |
| 69 | Not tested |
| 70 | 0.147 |

### Colonic ENaCgamma Expression Assay

The effect of a test compound can also be evaluated for potential therapeutic applications by a functional assay, in which the test compound blocks in vivo expression of a surrogate protein marker for mineralocorticoid receptor activation. In this assay aldosterone induced expression of colonic ENaCgamma is measured. Male Sprague-Dawley rats (225-250 g) (Harlan Sprague-Dawley Industries, Indianapolis, Ind.) were used in this assay. All animals were housed in a room with ambient temperature of 22±1° C. on a 12 hour light/dark cycle. Animals were allowed one week to acclimate and had free access to Teklad 22/5 rodent chow (Harlan Teklad, Madison, Wis.) and tap water ad libitum until the initiation of the study.

The rats were initially anesthetized with 5% Isoflurane (AErrane; Baxter, Inc., Deerfield, Ill.) delivered in 100% O2 (USP Medical Grade, Airgas-Mid America, Bowling Green, Ky.) using a VMS anesthesia instrument (Matrix Medical, Inc., Orchard Park, N.Y.) Once anesthetized, 1-2% Isoflurane was used to maintain anesthesia. The surgery site was shaved, scrubbed with Dial 4% CHG surgical scrub (Dial Corp., Phoenix, Ariz.), and sprayed with Betadine Aerosol topical antiseptic/bactericide spray (Perdue Frederick Co., Stamford, Conn.). and a bilateral adrenalectomy (ADX) was performed via the dorsal approach. The muscle layer was closed with 4-0 vicryl and skin wounds closed with surgical staples. The analgesic, Marcaine (0.25%) (Abbot Laboratories, Chicago, Ill.) was injected (0.1 mL, s.c.) at the incision site. Post-operative care included monitoring of the animals, which were placed on thermogenic heating pads during recovery from anesthesia until sternal recumbency and alertness were obtained. Animals were inspected daily for signs of distress and infection at the surgical site. ADX rats were given 0.9% NaCl in the drinking water to compensate the sodium deficiency induced by the ADX.

After 3 days of recovery from surgery, and following an overnight fast, rats were randomly assigned into five groups (n=5-9), including three treatment groups, one control group and one vehicle group. The vehicle and control groups were dosed with solution vehicle (10% EtOH, 70% PEG 400, 20% PBS); the rats in the treatment groups were dosed orally with test compounds at 1 mg/kg, dissolved in the solution vehicle. Aldosterone (5 ug/kg, Sigma, St. Louis, Mo.) was given to all treatment groups and the control group at 30 minutes post-dose. Blood and distal colon were collected at 2 hours post-dose. The rats were sacrificed with CO2 and animals were exsanguinated using an 18-gauge needle inserted into the heart. The distal colon was extracted and immediately placed in liquid N2 for later ENaCγ level determination. Blood was centrifuged for 15 minutes at 3000 rpm, 4° C. and serum collected and frozen at −80° C. until further analysis.

Frozen distal colon was powdered, lysed in Qiagen RLT buffer with chloroform, and the aqueous layer combined with 70% ethanol and purified over the Qiagen 96-well RNeasy system (Qiagen Inc, Valencia, Calif.). 5 ul reactions were prepared with the Bioimaek 2000 and Fx instruments, and Q-RT-PCR was performed using Qiagen one-step reagents. Thermocycling and data collection were performed on an ABI 7900 (Applied Biosystems, Foster City, Calif.). The comparative CT (threshold cycle) method of calculation was used for determining relative expression of mineralocorticoid receptor target genes; cyclophilin was used to normalize expression.

### Dahl SS Rat Blood Pressure Assay

The effect of a test compound on systemic blood pressure and microalbuminuria (urinary albumin creatinine ratio) can be evaluated in vivo, using a salt-dependent animal model of hypertension. Male Dahl salt-sensitive rats (225-250 g) are used in this assay. Animals are housed and acclimated under the same conditions stated in the colonic ENaCgamma assay above.

All animals are instrumented with radiotelemetry units (Data Sciences Inc., St. Paul, Minn.) for conscious, unrestricted SBP measurements. The rats are anesthetized with Isoflurane delivered in 100% O2 and a laparotomy is performed via midline incision using aseptic techniques. A radiotelemetry probe-flow catheter is inserted in the abdominal aorta between the renal arteries and the bifurcation of the iliac arteries and secured to the psoas muscle. The transmitter is sewn into the muscle layer, upon closure. The rats are given analgesics and provided post operative care.

After 5-7 days of recovery from surgery, baseline SBP is measured and all animals is then randomized to various treatment groups and compounds are continued for 21 days. All animals are placed on Teklad 92034 4% NaCl rodent chow (Harlan Teklad), which was maintained for 21 days. All compounds are dissolved in the appropriate vehicle. The vehicle group received vehicle and the compound treated groups are dosed at various concentrations with the compounds daily, via gavage. Compounds are also administered to the treated groups using an ad mixture incorporated into the 4% NaCl rodent chow at various concentrations (Research Diets, Inc., New Brunswick, N.J.).

Radiotelemetrized arterial SBP is measured with the DATAQUEST A.R.T. Version 3.0—Gold software (Data Sciences International, St. Paul, Minn.). The values represent the average of all data points collected from each animal, every minute for over a 24-hour period (6:00 a.m. to 6:00 a.m. the following day). SBP data is collected continuously over the course of the entire study (days 1-21).

Twenty-four hours prior to the termination of the study, animals are placed in metabolism caging and urine is collected at 24 hours. Animals are not fasted for the 24-hour period. After 21 days of treatment, animals are weighed with a Mettler PM6000 balance (Mettler-Toledo, Inc., Hightstown, N.J.) and anesthetized. Animals are exsanguinated and samples collected. Plasma and urine chemistries (e.g., albumin, creatinine and electrolytes) are analyzed according to standard procedures.

### SHR Blood Pressure Assay

The effect of a test compound on systemic blood pressure can be evaluated in vivo, using an salt-independent animal model of hypertension. Spontaneously hypertensive rats (SHR) (250-270 g) from Charles River Laboratories (Wilmington, Mass.) were used in this assay. Animals were housed and acclimated under the same conditions stated in the Dahl salt-sensitive rats above. Rats were pair housed under a 12-h light/dark cycle with free access to water and normal Purina rat chow (Purina Mills, Richmond, Ind.)

SHR rats were also implanted with radiotelemetry units (Data Sciences Inc., St. Paul, Minn.) for conscious, unrestricted SBP measurements using the same aseptic surgical techniques as those in the Dahl salt-sensitive rats above. After 5-7 days of recovery from surgery, baseline SBP were measured for 24 hours and all animals were then randomized to various vehicle and treatment groups. All animals were conscious and had access to normal rodent chow and water ad libitum while blood pressure was monitored continuously. All compounds were dissolved in the appropriate vehicle. The vehicle group received vehicle and the compound treated groups were dosed at various concentrations and frequencies with the compounds daily, via gavage, for 14 days.

Radiotelemetrized arterial SBP was measured with the DATAQUEST A.R.T. Version 3.0—Gold software (Data Sciences International, St. Paul, Minn.). The values represent the average of all data points collected from each animal, every minute for over a 24-hour period (6:00 a.m. to 6:00 a.m. the following day). SBP data is collected continuously over the course of the entire study (days 1-21).

Twenty-four hours prior to the termination of the study, and after 14 days of treatment, animals were weighed with a Mettler PM6000 balance (Mettler-Toledo, Inc., Hightstown, N.J.) and anesthetized. Animals were then exsanguinated and plasma samples collected. Plasma chemistries (e.g., Alodosterone and electrolytes) were analyzed according to standard procedures.

Administration of the compounds of this invention can be via any method which delivers a compound of this invention systemically and/or locally. These methods include oral routes, parenteral, intraduodenal routes, etc. Generally, the compounds of this invention are administered orally, but parenteral administration (e.g., intravenous, intramuscular, subcutaneous or intramedullary) may be utilized, for example, where oral administration is inappropriate for the target or where the patient is unable to ingest the drug.

For administration to human patients, oral daily dose of the compounds herein may be in the range 1 mg to 500 mg depending, of course, on the mode of administration. An oral daily dose is in the range of 3 mg to 250 mg may be used. A further oral daily dose is in the range of 5 mg to 180 mg. The total daily dose may be administered in single or divided doses and may, at the physician's discretion, fall outside of the typical ranges given herein.

For convenience the compounds of the present invention can be administered in a unit dosage form. If desired, multiple doses per day of the unit dosage form can be used to increase the total daily dose. The unit dosage form, for example, may be a tablet or capsule containing about 0.01, 0.05, 0.1, 0.5, 1, 5, 10, 15, 20, 25, 30, 35, 40, 45, 50, 55, 60, 65, 70, 75, 80, 85, 90, 95, 100, 125, 150, 175, 200, 250 or 500 mg of the compound of the present invention. In one embodiment, the unit dosage form contains from about 0.01 mg to about 500 mg of the compound of the present invention. In another embodiment, the unit dosage form contains from about 0.05 mg to about 250 mg of the compound of the present invention. In another embodiment, the unit dosage form contains from about 0.1 mg to about 200 mg of the compound of the present invention. In another embodiment, the unit dosage form contains from about 0.5 mg to about 150 mg of the compound of the present invention.

These compounds may also be administered to animals other than humans for example, for the indications detailed above. The precise dosage administered of each active ingredient will vary depending upon any number of factors, including but not limited to, the type of animal and type of disease state being treated, the age of the animal, and the route(s) of administration.

A dosage of the combination pharmaceutical agents to be used in conjunction with the Formula I compounds is used that is effective for the indication being treated. Such dosages can be determined by standard assays such as those referenced above and provided herein. The combination agents may be administered simultaneously or sequentially in any order.

These dosages are based on an average human subject having a weight of about 60 kg to 70 kg. The physician will readily be able to determine doses for subjects whose weight falls outside this range, such as infants and the elderly.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

The present invention further comprises use of a compound of Formulae I or II for use as a medicament (such as a unit dosage tablet or unit dosage capsule). In another embodiment, the present invention comprises the use of a compound of Formulae I or II for the manufacture of a medicament (such as a unit dosage tablet or unit dosage capsule) to treat one or more of the conditions previously identified in the above sections discussing methods of treatment. In one embodiment, the condition is hypertension. In another embodiment the condition is diabetic nephropathy.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The compounds described herein may be administered as a formulation comprising a pharmaceutically effective amount of a compound of Formula I, in association with one or more pharmaceutically acceptable excipients. The term “carrier” or “excipient” herein means any substance, not itself a therapeutic agent, used as a diluent, adjuvant, or vehicle for delivery of a therapeutic agent to a subject or added to a pharmaceutical composition to improve its handling or storage properties or to permit or facilitate formation of a solid dosage form such a tablet, capsule, or a solution or suspension suitable for oral parenteral, intradermal, subcutaneous, or topical application. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutical acceptable materials. Examples of excipients and their use may be found in Remington's Pharmaceutical Sciences, 20th Edition (Lippincott Williams & Wilkins, 2000).The choice of excipient will to a large extent depend on factors such as the particular mode of administration, the effect of the excipient on solubility and stability, and the nature of the dosage form.

The compounds herein may be formulated for oral, buccal, intranasal, parenteral (e.g., intravenous, intramuscular or subcutaneous) or rectal administration or in a form suitable for administration by inhalation. The compounds of the invention may also be formulated for sustained delivery.

Methods of preparing various pharmaceutical compositions with a certain amount of active ingredient are known, or will be apparent in light of this disclosure, to those skilled in this art. For examples of methods of preparing pharmaceutical compositions see Remington's Pharmaceutical Sciences, 20th Edition (Lippincott Williams & Wilkins, 2000).

Pharmaceutical compositions according to the invention may contain 0.1%-95% of the compound(s) of this invention, preferably 1%-70%. In any event, the composition or Formulation to be administered will contain a quantity of a compound(s) according to the invention in an amount effective to treat the disease/condition of the subject being treated, e.g., atherosclerosis.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of Formula I a prodrug thereof or a salt of such compound or prodrug and a second compound as described above. The kit comprises means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of Formula I compound can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compounds of this invention either alone or in combination with each other or other compounds generally will be administered in a convenient formulation. The following formulation examples only are illustrative and are not intended to limit the scope of the present invention.

In the formulations which follow, “active ingredient” means a compound of this invention.

### General Experimental Procedures

All chemicals, reagents and solvents were purchased from commercial sources when available and used without further purification. Proton nuclear magnetic spectroscopy (1H-NMR) was recorded with 400 and 500 MHz Varian spectrometers. Chemical shifts are expressed in parts per million downfield from tetramethylsilane. The peak shapes are denoted as follows: s, singlet; d, doublet; t, triplet; q, quartet; m, multiplet; bs, broad singlet. Mass spectrometry (MS) was performed via atmospheric pressure chemical ionization (APCI) or electron scatter (ES) ionization sources. Silica gel chromatography was performed primarily using a medium pressure Biotage or ISCO systems using columns pre-packaged by various commercial vendors including Biotage and ISCO. Preparative scale separations were performed using high pressure liquid chromatography (HPLC) or supercritical fluid chromatography (SFC). Microanalyses were performed by Quantitative Technologies Inc. and were within 0.4% of the calculated values. The terms “concentrated” and “evaporated” refer to the removal of solvent at reduced pressure on a rotary evaporator with a bath temperature less than 60° C. The abbreviation “min” and “h” stand for “minutes” and “hours” respectively.

### EXAMPLES

Preparation 1: ethyl 3-cyclopentylacrylate

Sodium ethoxide 21 wt % in ethanol (600 mL, 1.6 mol) was added dropwise over 1 hour to a 0° C. solution of triethyl phosphonoacetate (343 g, 1.5 mol) in 2-methyltetrahydrofuran (3.1 L). The reaction was allowed to stir for 30 min and cyclopentancarboxaldehyde (163 mL, 1.5 mol) was added dropwise over 1 h. The reaction was then allowed to warm to room temperature for 16 h. The reaction mixture was filtered through celite to remove insolubles. The filtrate was extracted 3 times with water (750 mL), 3 times with saturated aqueous sodium bicarbonate (750 mL), and 2 times with saturated aqueous ammonium chloride (500 mL). The organic layer was dried over sodium sulfate, filtered and concentrated to an orange oil. The crude oil was purified via silica chromatography using 5% ethyl acetate in heptane to yield the title compound (218.6 g, 83%) as colorless oil. 1H NMR (400 MHz, DMSO-d₆) δ ppm 1.12 (1H, d, J=5.9 Hz), 1.21 (1H, m), 1.20 (2H, t, J=7.0 Hz), 1.34 (1H, ddd, J=12.3, 5.9, 2.1 Hz), 1.56 (1H, dt, J=7.3, 3.6 Hz), 1.64 (1H, ddd, J=8.8, 4.7, 4.5 Hz), 1.80 (2H, m), 1.93 (1H, m), 2.60 (1H, m, J=8.0, 8.0, 8.0, 8.0, 7.8 Hz), 3.31 (1H, s), 4.10 (2H, q, J=7.0 Hz), 5.82 (1H, d, J=15.6 Hz), 6.86 (1H, dd, J=15.6, 7.8 Hz)

Preparation 2: 4-(3-chloro-5-cyclopentyl-4,5-dihydro-1H-pyrazol-1-yl)-2-methylbenzonitrile
Step 1: 4-(5-cyclopentyl-3-oxopyrazolidin-1-yl)-2-methylbenzonitrile

To a solution of ethyl 3-cyclopentylacrylate (Preparation 1, 150.0 g, 0.89 mol) and 4-hydrazinyl-2-methylbenzonitrile hydrochloride (from WO 2008/053300, 157.5 g, 1.07 mol) in tetrahydrofuran (20 L) was added potassium t-butoxide (208.4 g, 1.78 mol) and the reaction then heated to reflux for 3 h. The reaction was cooled to room temperature and was quenched by adding 1N HCl (1.2 L, to pH=2). The mixture was stirred for 2 h and phase separated. The aqueous phase was extracted twice with ethyl acetate (500 mL). The combined organic layers were washed 3 times with water (500 mL) until the pH was neutral (˜7), washed with brine, dried over sodium sulfate and filtered. The organic layer was concentrated to a solid. The material was triturated by diluting in a 2:1 solution of methyl tert-butylether/heptane and heating to reflux. The slurry was cooled to room temperature for 2 h. The solid was filtered to yield the title compound (190.3 g, 79%). 1H NMR (400 MHz, DMSO-d₆) δ ppm 1.13-1.30 (m, 1H) 1.35-1.82 (m, 7H) 2.02 (d, J=16.80 Hz, 1H) 2.07-2.17 (m, 1H) 2.42 (s, 3H) 2.82 (dd, J=16.51, 8.30 Hz, 1H) 4.07 (t, J=8.11 Hz, 1H) 6.86 (d, J=8.60 Hz, 1H) 6.93 (s, 1H) 7.60 (d, J=8.60 Hz, 1H) 10.27 (s, 1H).

Step 2: 4-(3-chloro-5-cyclopentyl-4,5-dihydro-1H-pyrazol-1-yl)-2-methylbenzonitrile

A mixture of 4-(5-cyclopentyl-3-oxopyrazolidin-1-yl)-2-methylbenzonitrile (250 g, 0.84 mol) in acetonitrile (11 L) was treated with phosphoryl chloride (85 mL. 0.92 mol) and heated to 80° C. for 2 h. The reaction was stirred overnight at room temperature. The reaction was concentrated to dark brown solid that were dissolved in dichloromethane and washed with saturated sodium bicarbonate, brine, dried over sodium sulfate and filtered. The organic layer was concentrated. The residue was purified by silica gel column chromatography eluting with a gradient of 5%-40% ethyl acetate/heptane to yield the title compound (214 g, 89%) as a solid. ¹H NMR (400 MHz, CHLOROFORM-d) δ ppm 1.15-1.25 (m, 2H) 1.48-1.72 (m, 5H) 1.73-1.86 (m, 1H) 2.43-2.58 (m, 4 H) 2.87 (dd, J=17.98, 4.69 Hz, 1H) 3.31 (dd, J=17.98, 11.53 Hz, 1H) 4.60 (dt, J=11.48, 4.42 Hz, 1H) 6.79 (dd, J=8.60, 2.34 Hz, 1H) 6.94 (d, J=2.34 Hz, 1H) 7.44 (d, J=8.79 Hz, 1H).

Preparation 3: (R)-4-(3-chloro-5-cyclopentyl-4,5-dihydro-1H-pyrazol-1-yl)-2-methylbenzonitrile

The title compound was prepared from 4-(3-chloro-5-cyclopentyl-4,5-dihydro-1H-pyrazol-1-yl)-2-methylbenzonitrile (Preparation 2) using chiral SFC; Column AD-H 30×250 mm column, 10/90 isopropanol/carbon dioxide, 70 mL/min. Second eluting peak: chiral HPLC t_{R}=1.8 min (Chiralpak AD-H 30×250 mm column, 20/80 isopropanol/carbon dioxide). 1H NMR (400 MHz, DMSO-d₆) δ ppm 1.18 (1H, m), 1.15 (1H, d, J=9.8 Hz), 1.40 (1H, dd, J=17.2, 9.4 Hz), 1.41 (1H, m), 1.48 (1H, d, J=7.4 Hz), 1.59 (2 H, dd, J=8.8, 3.3 Hz), 1.55 (1H, m), 1.71 (1H, dd, J=8.0, 4.1 Hz), 2.43 (3H, m), 2.95 (1 H, dd, J=18.4, 4.3 Hz), 3.32 (1H, s), 3.48 (1H, dd, J=18.4, 11.3 Hz), 4.78 (1H, dt, J=11.3, 4.3 Hz), 7.00 (1H, d), 7.52 (1H, d).

The absolute stereochemistry for the above compound was determined in part by assessment of the absolute stereochemistry for the Preparation 7 intermediate as determined below (single crystal X-ray analysis) which is an analogous intermediate to the intermediate of Preparation 3.

Preparation 4: 2-chloro-4-(3-chloro-5-cyclopentyl-4,5-dihydro-1H-pyrazol-1-yl)benzonitrile
Step 1: 2-chloro-4-(5-cyclopentyl-3-oxopyrazolidin-1-yl)benzonitrile

To a solution of ethyl 3-cyclopentylacrylate (Preparation 1, 10.0 g, 59.4 mmol) and 2-chloro-4-hydrazinylbenzonitrile hydrochloride (from WO 2008/053300, 9.96 g, 59.4 mmol) in ethanol (120 mL) was added dropwise 21% sodium ethoxide solution in ethanol (55.5 mL) and the mixture was heated to 85° C. overnight. The reaction was cooled to 15° C. and was quenched by adding 1N HCl (˜20 mL, to pH ˜4-5), which gave a yellow precipitate. The mixture was diluted with water (500 mL) and the solid was collected by filtration. The solid was further rinsed with water, isolated and dried in oven vacuum overnight to yield the title compound (9.0 g, 52.3%). 1H NMR (400 MHz, DMSO-d₆) δ ppm 1.20 (1H, d, J=9.0 Hz), 1.38 (1H, br. s.), 1.54 (2H, td, J=7.4, 3.5 Hz), 1.66 (1H, t, J=7.4 Hz), 1.65 (1H, br. s.), 1.75 (2H, dd, J=11.5, 7.2 Hz), 2.08 (1H, d, J=16.8 Hz), 2.92 (1H, dd, J=16.6, 8.4 Hz), 3.32 (1H, s), 4.24 (1H, t, J=8.0 Hz), 6.97 (1 H, dd, J=9.0, 2.0 Hz), 7.09 (1H, d, J=2.3 Hz), 7.77 (1H, d, J=8.6 Hz), 10.46 (1H, s).

Step 2: 2-chloro-4-(3-chloro-5-cyclopentyl-4,5-dihydro-1H-pyrazol-1-yl)benzonitrile

The title compound was prepared by the method used for Preparation 2, Step 2 from 2-chloro-4-(5-cyclopentyl-3-oxopyrazolidin-1-yl)benzonitrile (9.0 g, 31.1 mmol). 8.50 g isolated (88.8%) as a solid. 1H NMR (400 MHz, DMSO-d₆) δ ppm 1.12 (2H, m), 1.42 (2H, m), 1.57 (2H, m), 1.71 (1H, dt, J=11.6, 7.7 Hz), 2.39 (1H, td, J=6.9, 3.7 Hz), 2.99 (1 H, dd, J=18.4, 3.9 Hz), 3.34 (1H, br. s.), 3.51 (1H, dd, J=18.4, 11.3 Hz), 4.85 (1H, dt, J=11.3, 4.3 Hz), 7.03 (1H, dd, J=9.0, 2.3 Hz), 7.18 (1H, d, J=2.3 Hz), 7.70 (1H, m).

Preparation 5: (R)-2-chloro-4-(3-chloro-5-cyclopentyl-4,5-dihydro-1H-pyrazol-1-yl)benzonitrile

The title compound was prepared from 2-chloro-4-(3-chloro-5-cyclopentyl-4,5-dihydropyrazol-1-yl)benzonitrile prepared in Preparation 4 using chiral SFC; Column: AD-H 50×250 mm, 20% methanol/carbon dioxide, 220 mL/min. Second eluting peak: chiral HPLC t_{R}=3.88 min (Chiralpak AD-H 30×250 mm column, 20% methanol/carbon dioxide). 1H NMR (400 MHz, DMSO-d₆) δ ppm 1.03-1.22 (m, 2H) 1.33-1.65 (m, 5H) 1.66-1.78 (m, 1H) 2.32-2.46 (m, 1H) 2.99 (dd, J=18.53, 4.03 Hz, 1H) 3.51 (dd, J=18.26, 11.28 Hz, 1H) 4.85 (dt, J=11.41, 4.16, 4.03 Hz, 1H) 7.03 (dd, J=8.86, 2.42 Hz, 1H) 7.18 (d, J=2.42 Hz, 1H) 7.70 (d, J=8.59 Hz, 1H).

Preparation 6: 4-(3-chloro-5-cyclopentyl-4,5-dihydro-1H-pyrazol-1-yl)-2-methoxybenzonitrile
Step 1: 2-(2-(4-cyano-3-methoxyphenyl)hydrazono)acetic acid

To a solution of glyoxylic acid (1.22 g, 13.3 mmol) in water (200 mL) was added 4-hydrazinyl-2-methoxybenzonitrile (WO 2008/053300, 2.50 g, 13.3 mmol). The mixture was stirred for 16 h. The reaction mixture was filtered and washed with two portions of water and air dried for 48 hours to give 2.74 g (100%) of the title compound as a solid. 1H NMR (400 MHz, DMSO-d₆) δ ppm 3.37 (s, 1H), 3.89 (s, 3H), 6.72 (dd, J=8.46, 1.75 Hz, 1H), 6.85 (d, J=1.61 Hz, 1H), 7.23 (s, 1H), 7.56 (d, J=8.59 Hz, 1H), 11.55 (s, 1H).

Step 2: 4-(3-chloro-5-cyclopentyl-4,5-dihydro-1H-pyrazol-1-yl)-2-methoxybenzonitrile

2-(2-(4-cyano-3-methoxyphenyl)hydrazono)acetic acid (2.74 g, 12.5 mmol), N-chlorosuccinimide (3.44 g, 25.8 mmol), sodium bicarbonate (2.16 g, 25.7 mmol), vinylcyclopentane (3.0 g, 31 mmol) and ethyl acetate (150 mL) were combined and stirred for 16 h. The reaction was heated to 80° C. for 7 h and then allowed to cool to ambient temperature. To the reaction was added water (150 mL). The phases were cut and the organic phase was concentrated. The residue was purified by silica gel chromatography with a gradient of 0%-25% ethyl acetate/heptane to give of the title compound (1.0 g, 26%) as a solid. 1H NMR (400 MHz, CHLOROFORM-d) δ ppm 1.45-1.88 (m, 6H) 2.44-2.61 (m, 1H) 2.88 (dd, J=17.99, 4.30 Hz, 1H) 3.33 (dd, J=17.99, 11.55 Hz, 1H) 3.92 (s, 3H) 3.92-3.95 (m, 2H) 4.54-4.68 (m, 1H) 6.37 (dd, J=8.73, 2.01 Hz, 1H) 6.72-6.77 (m, 1H) 7.34-7.42 (m, 1H).

Preparation 7: (R)-6-(3-chloro-5-cyclopentyl-4,5-dihydro-1H-pyrazol-1-yl)-2-methylnicotinonitrile
Step 1: 5-cyclopentylpyrazolidin-3-one

Ethyl 3-cyclopentylacrylate (Preparation 1, 450 g, 2.67 mol) was dissolved in ethanol (8.78 L) and added dropwise to a solution of hydrazine hydrate (129 mL, 133g, 2.67 mol) in ethanol (8.78 L). The solution was stirred at ambient temperature for 1 h then heated to reflux for 48 h. The reaction was then concentrated to afford a gummy yellow solid which was diluted with hexanes (1 L) and stirred for 16 h at room temperature. The resulting slurry was diluted with diethyl ether (1 L) and stirred at room temperature for 1 h. The solid was filtered off to give the title compound (260.9 g, 63%) as a beige solid. 1H NMR (400 MHz, DMSO-d₆) δ ppm 1.08-1.18 (m, 1H) 1.21-1.31 (m, 1H) 1.41-1.60 (m, 4H) 1.60-1.71 (m, 2H) 1.88 (td, J=16.38, 8.06 Hz, 1H) 2.00 (dd, J=15.84, 8.32 Hz, 1H) 2.29 (dd, J=15.84, 7.25 Hz, 1H) 3.15 (ddd, J=16.58, 8.59, 8.39 Hz, 1H) 5.08 (d, J=8.86 Hz, 1H) 8.91 (br. s., 1H).

Step 2: 6-(5-cyclopentyl-3-oxopyrazolidin-1-yl)-2-methylnicotinonitrile

In a microwave reaction vessel were combined 5-cyclopentylpyrazolidin-3-one (11 g, 72.1 mmol), 6-chloro-2-methylnicotinonitrile (10 g, 65.4 mmol) and water (35 mL). The mixture was heated to 150° C. for 30 min in a microwave reactor. The reaction was cooled to room temperature and the resulting solid was isolated by vacuum filtration, rinsed with water (75 mL) and dried to give the title compound as a light brown solid (15.0 g, 87%). 1H NMR (400 MHz, DMSO-d₆) δ ppm 1.24 (dd, J=12.30, 7.23 Hz, 1H) 1.38-1.65 (m, 6H) 1.65-1.76 (m, 1H) 2.03-2.19 (m, 2H) 2.48 (s, 3H) 2.89 (dd, J=16.80, 8.98 Hz, 1H) 4.74 (t, J=8.01 Hz, 1H) 6.67 (d, J=8.59 Hz, 1H) 7.89 (d, J=8.98 Hz, 1H) 10.61 (br. s., 1H).

Step 3: 6-(3-chloro-5-cyclopentyl-4,5-dihydro-1H-pyrazol-1-yl)-2-methylnicotinonitrile

The title compound was prepared by the method used for Preparation 2, Step 2 from 6-(5-cyclopentyl-3-oxopyrazolidin-1-yl)-2-methylnicotinonitrile (140 g, 518 mmol). 131.4 g of the title compound was isolated (79%) as a rose colored solid. 1H NMR (400 MHz, DMSO-d₆) δ ppm 1.20 (2H, m), 1.51 (1H, m), 1.45 (2H, dd, J=10.7, 6.8 Hz), 1.57 (1H, m), 1.60 (2H, dd, J=8.4, 5.7 Hz), 2.45 (3H, s), 2.69 (1H, ddd, J=4.9, 2.3, 2.1 Hz), 2.96 (1H, dd, J=18.4, 4.7 Hz), 3.48 (1H, dd, J=18.4, 11.7 Hz), 4.93 (1H, ddd, J=11.5, 4.9, 4.7 Hz), 6.94 (1H, d, J=8.6 Hz), 7.82 (1H, d, J=8.6 Hz).

Step 4: (R)-6-(3-chloro-5-cyclopentyl-4,5-dihydro-1H-pyrazol-1-yl)-2-methylnicotinonitrile

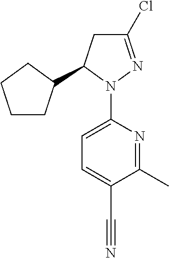

The title compound was prepared from 6-(3-chloro-5-cyclopentyl-4,5-dihydro-1H-pyrazol-1-yl)-2-methylnicotinonitrile using chiral SFC (Chiralpak AD-H 30×250 mm column, 10% isopropanol/carbon dioxide, 70 mL/min First eluting peak: chiral HPLC t_{R}⁼1.76 (Chiralpak AD-H, 20% isopropanol/carbon dioxide). 1H NMR (400 MHz, DMSO-d₆) δ ppm 1.08-1.26 (m, 2H) 1.35-1.67 (m, 6H) 2.47 (s, 3H) 2.60-2.72 (m, 1H) 2.92 (dd, J=18.44, 4.78 Hz, 1H) 3.44 (dd, J=18.44, 11.41 Hz, 1H) 4.86-4.94 (m, 1H) 6.91 (d, 1H) 7.79 (s, 1H).

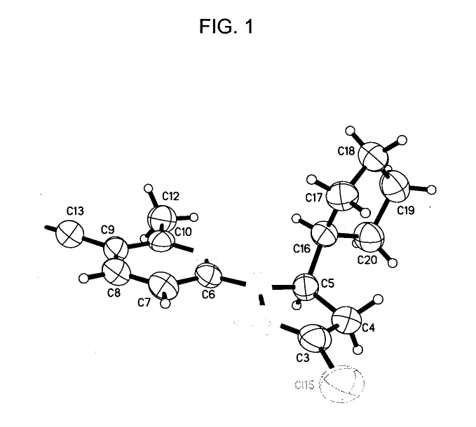

Single Crystal X-Ray Analysis for (R)-6-(3-chloro-5-cyclopentyl-4,5-dihydro-1H-pyrazol-1-yl)-2-methylnicotinonitrile (FIG. 1): A representative crystal was surveyed and a 1A data set (maximum sin Θ/λ=0.5) was collected on a Bruker APEX II/R diffractometer. Friedel pairs were collected in order to facilitate the determination of the absolute configuration. Atomic scattering factors were taken from International Tables for Crystallography, Vol. C, pp. 219, 500, Kluwer Academic Publishers, 1992. All crystallographic calculations were facilitated by the SHELXTL (Version 5.1, Bruker AXS, 1997) system. All diffractometer data were collected at room temperature. Pertinent crystal, data collection, and refinement are summarized in Table 2.

A trial structure was obtained by direct methods. This trial structure refined routinely. Hydrogen positions were calculated wherever possible. The methyl hydrogens were located by difference Fourier techniques and then idealized. The hydrogen parameters were added to the structure factor calculations but were not refined. The shifts calculated in the final cycles of least squares refinement were all less than 0.1 of the corresponding standard deviations. The final R-index was 3.56%. A final difference Fourier revealed no missing or misplaced electron density.

The refined structure was plotted using the SHELXTL plotting package (FIG. 1). The absolute configuration was determined by the method of Flack (Acta Crystallogr., A39, 876, 1983). Coordinates, anisotropic temperature factors, distances and angles are available as supplementary material (Tables 2-6).

TABLE 2
Crystal data and structure refinement for (R)-6-(3-chloro-5-cyclopentyl-
4,5-dihydro-1H-pyrazol-1-yl)-2-methylnicotinonitrile

| | |
|---|---|
| Identification code | I731 |
| Empirical formula | C₁₅H₁₇N₄Cl |
| Formula weight | 288.78 |
| Temperature | 296(2) K |
| Wavelength | 1.54178 Å |
| Crystal system | Orthorhombic |
| Space group | P2(1)2(1)2(1) |

| | | |
|---|---|---|
| Unit cell dimensions | a = 6.1007(4) Å | α = 90°. |
| | b = 11.6792(6) Å | β = 90°. |
| | c = 20.4798(10) Å | γ = 90°. |
| Volume | 1459.21(14) Å³ | |
| Z | 4 | |
| Density (calculated) | 1.314 Mg/m³ | |
| Absorption coefficient | 2.273 mm⁻¹ | |
| F(000) | 608 | |
| Crystal size | 0.18 × 0.12 × 0.06 mm³ | |
| Theta range for data collection | 4.36 to 50.08°. | |
| Reflections collected | 4668 | |
| Independent reflections | 1303 [R(int) = 0.0355] | |
| Completeness to theta = 50.08° | 90.6% | |
| Absorption correction | Empirical Absorption Correction | |
| Max. and min. transmission | 0.8757 and 0.6851 | |
| Refinement method | Full-matrix least-squares on F² | |
| Data/restraints/parameters | 1303/0/182 | |
| Goodness-of-fit on F² | 1.085 | |
| Final R indices [I > 2sigma(I)] | R1 = 0.0356, wR2 = 0.0912 | |
| Absolute structure parameter | 0.03(3) | |
| Extinction coefficient | 0.0005(4) | |
| Largest diff. peak and hole | 0.152 and −0.142 e.Å⁻³ | |

TABLE 3
Atomic coordinates (×10⁴) and equivalent isotropic displacement
parameters (Å² × 10³) for (R)-6-(3-chloro-5-cyclopentyl-
4,5-dihydro-1H-pyrazol-1-yl)-2-methylnicotinonitrile. U(eq) is defined
as one third of the trace of the orthogonalized Uᵢⱼtensor.

| | x | y | z | U(eq) |
|---|---|---|---|---|
| N(1) | 600(5) | 4453(2) | 9317(1) | 55(1) |
| N(2) | 1992(5) | 4033(2) | 8832(1) | 59(1) |
| C(3) | 1032(7) | 4160(3) | 8293(2) | 62(1) |
| C(4) | −1172(7) | 4693(3) | 8315(2) | 65(1) |
| C(5) | −1537(6) | 4849(3) | 9055(1) | 51(1) |
| C(6) | 1020(7) | 4136(3) | 9949(2) | 46(1) |
| C(7) | 2960(7) | 3572(3) | 10117(2) | 58(1) |
| C(8) | 3287(7) | 3312(3) | 10755(2) | 62(1) |
| C(9) | 1746(6) | 3592(3) | 11220(2) | 52(1) |
| C(10) | −148(6) | 4152(3) | 11019(2) | 51(1) |
| N(11) | −535(5) | 4400(2) | 10390(1) | 51(1) |
| C(12) | −1908(7) | 4489(3) | 11495(2) | 68(1) |
| C(13) | 2103(7) | 3352(3) | 11898(2) | 67(1) |
| N(14) | 2425(7) | 3139(3) | 12433(2) | 97(1) |
| Cl(15) | 2264(2) | 3748(1) | 7587(1) | 98(1) |
| C(16) | −2143(6) | 6066(2) | 9260(1) | 49(1) |
| C(17) | −569(7) | 7018(3) | 9076(2) | 62(1) |
| C(18) | −1963(8) | 8096(3) | 9150(2) | 73(1) |
| C(19) | −4290(8) | 7753(3) | 8995(2) | 73(1) |
| C(20) | −4350(7) | 6445(3) | 8978(2) | 70(1) |

TABLE 4
Bond Lengths [Å] and angles [°] for (R)-6-(3-chloro-5-cyclopentyl-
4,5-dihydro-1H-pyrazol-1-yl)-2-methylnicotinonitrile.

| | | | |
|---|---|---|---|
| N(1)—C(6) | 1.371(4) | C(8)—C(9) | 1.379(5) |
| N(1)—N(2) | 1.396(4) | C(9)—C(10) | 1.390(5) |
| N(1)—C(5) | 1.483(5) | C(9)—C(13) | 1.431(5) |
| N(2)—C(3) | 1.259(4) | C(10)—N(11) | 1.341(4) |
| C(3)—C(4) | 1.482(6) | C(10)—C(12) | 1.504(5) |
| C(3)—Cl(15) | 1.699(4) | C(13)—N(14) | 1.141(4) |
| C(4)—C(5) | 1.543(4) | C(16)—C(17) | 1.517(5) |
| C(5)—C(16) | 1.527(4) | C(16)—C(20) | 1.531(5) |
| C(6)—N(11) | 1.345(4) | C(17)—C(18) | 1.527(5) |
| C(6)—C(7) | 1.398(5) | C(18)—C(19) | 1.508(6) |
| C(7)—C(8) | 1.356(4) | C(19)—C(20) | 1.529(5) |
| C(6)—N(1)—N(2) | 117.6(3) | C(8)—C(9)—C(10) | 118.2(3) |
| C(6)—N(1)—C(5) | 126.2(3) | C(8)—C(9)—C(13) | 121.3(4) |
| N(2)—N(1)—C(5) | 112.8(2) | C(10)—C(9)—C(13) | 120.4(3) |
| C(3)—N(2)—N(1) | 107.4(3) | N(11)—C(10)—C(9) | 122.2(3) |
| N(2)—C(3)—C(4) | 116.4(3) | N(11)—C(10)—C(12) | 116.1(3) |
| N(2)—C(3)—Cl(15) | 120.5(3) | C(9)—C(10)—C(12) | 121.6(3) |
| C(4)—C(3)—Cl(15) | 123.1(3) | C(10)—N(11)—C(6) | 118.1(3) |
| C(3)—C(4)—C(5) | 102.1(3) | N(14)—C(13)—C(9) | 178.2(4) |
| N(1)—C(5)—C(16) | 113.8(3) | C(17)—C(16)—C(5) | 117.4(3) |
| N(1)—C(5)—C(4) | 101.1(3) | C(17)—C(16)—C(20) | 104.5(3) |
| C(16)—C(5)—C(4) | 114.5(3) | C(5)—C(16)—C(20) | 112.2(3) |
| N(11)—C(6)—N(1) | 116.1(3) | C(16)—C(17)—C(18) | 103.1(3) |
| N(11)—C(6)—C(7) | 122.7(3) | C(19)—C(18)—C(17) | 106.5(3) |
| N(1)—C(6)—C(7) | 121.2(3) | C(18)—C(19)—C(20) | 107.0(3) |
| C(8)—C(7)—C(6) | 117.8(3) | C(19)—C(20)—C(16) | 105.0(3) |
| C(7)—C(8)—C(9) | 120.9(4) | | |

Symmetry transformations used to generate equivalent atoms:

TABLE 5
Anisotropic displacement parameters (Å² × 10³) for (R)-6-(3-chloro-5-
cyclopentyl-4,5-dihydro-1H-pyrazol-1-yl)-2-methylnicotinonitrile.
The anisotropic displacement factor exponent takes the
form: −2π²[h² a*²U₁₁ + . . . + 2 h k a* b* U₁₂]

| | U₁₁ | U₂₂ | U₃₃ | U₂₃ | U₁₃ | U₁₂ |
|---|---|---|---|---|---|---|
| N(1) | 63(2) | 55(2) | 48(2) | 3(1) | 4(2) | 9(2) |
| N(2) | 69(2) | 49(2) | 58(2) | 3(1) | 18(2) | 3(2) |
| C(3) | 83(3) | 45(2) | 57(2) | 1(2) | 9(2) | −7(2) |
| C(4) | 79(3) | 52(2) | 64(2) | −6(2) | −9(2) | 8(2) |
| C(5) | 52(3) | 50(2) | 51(2) | 4(2) | −5(2) | −2(2) |
| C(6) | 51(3) | 37(2) | 51(2) | 4(1) | 4(2) | −4(2) |
| C(7) | 59(3) | 49(2) | 66(2) | −2(2) | 3(2) | 9(2) |
| C(8) | 70(3) | 44(2) | 71(3) | 1(2) | −10(2) | 3(2) |
| C(9) | 55(3) | 43(2) | 57(2) | 4(2) | −10(2) | −3(2) |
| C(10) | 65(3) | 38(2) | 50(2) | −1(2) | −2(2) | −13(2) |
| N(11) | 57(2) | 41(1) | 53(2) | 1(1) | 0(2) | −4(2) |
| C(12) | 82(3) | 65(2) | 58(2) | 1(2) | 6(2) | −4(2) |
| C(13) | 77(3) | 54(2) | 71(3) | 2(2) | −13(2) | 0(2) |
| N(14) | 119(3) | 104(2) | 67(2) | 12(2) | −24(2) | 10(2) |
| Cl(15) | 136(1) | 89(1) | 70(1) | −9(1) | 35(1) | 1(1) |
| C(16) | 48(3) | 46(2) | 53(2) | 1(1) | 1(2) | 2(2) |
| C(17) | 58(3) | 62(2) | 64(2) | −6(2) | 4(2) | 1(2) |
| C(18) | 98(4) | 53(2) | 68(2) | 3(2) | −3(2) | 4(2) |
| C(19) | 77(3) | 66(2) | 75(3) | 7(2) | 4(2) | 11(2) |
| C(20) | 61(3) | 65(2) | 85(3) | −1(2) | −7(2) | 10(2) |

TABLE 6
Hydrogen coordinates (×10⁴) and isotropic displacement
parameters (Å² × 10³) for (R)-6-(3-chloro-5-cyclopentyl-
4,5-dihydro-1H-pyrazol-1-yl)-2-methylnicotinonitrile.

| | x | y | z | U(eq) |
|---|---|---|---|---|
| H(4A) | −2273 | 4194 | 8126 | 80 |
| H(4B) | −1189 | 5422 | 8088 | 80 |
| H(5A) | −2690 | 4323 | 9200 | 80 |
| H(7A) | 3993 | 3381 | 9801 | 80 |
| H(8A) | 4568 | 2940 | 10880 | 80 |
| H(12A) | −3080 | 4862 | 11266 | 80 |
| H(12B) | −1308 | 5004 | 11814 | 80 |
| H(12C) | −2460 | 3818 | 11710 | 80 |
| H(16A) | −2281 | 6070 | 9737 | 80 |
| H(17A) | −56 | 6930 | 8630 | 80 |
| H(17B) | 684 | 7035 | 9367 | 80 |
| H(18A) | −1864 | 8389 | 9592 | 80 |
| H(18B) | −1468 | 8686 | 8851 | 80 |
| H(19A) | −4729 | 8064 | 8575 | 80 |
| H(19B) | −5281 | 8041 | 9326 | 80 |
| H(20A) | −4523 | 6170 | 8534 | 80 |
| H(20B) | −5551 | 6155 | 9240 | 80 |

Preparation 8: methyl 6-(3-cyclopentylacryloyl)-2-methoxynicotinate
Step 1: methyl 6-acetyl-2-methoxynicotinate

To a stirred solution of 6-acetyl-2-hydroxynicotinic acid (Tetrahedron (1990), 46 (23), 7693, 2.00 g, 11 mmol) in N,N-dimethylformamide (30 mL) was added cesium carbonate (10.80 g, 33.1 mmol) followed by iodomethane (3.46 g, 24.3 mmol). The resulting mixture was then stirred at room temperature under nitrogen for 16 h. The mixture was filtered and washed with ethyl acetate. The filtrate was concentrated and purified by silica gel column chromatography eluting with a gradient of 5%-25% ethyl acetate/heptane to obtain the title compound (1.2 g, 52%) as a solid. 1H NMR (500 MHz, CHLOROFORM-d) δ ppm 1.27 (1H, t, J=7.2 Hz), 1.57 (4H, s), 2.71 (2H, s), 3.94 (2H, s), 4.12 (2H, s).

Step 2: methyl 6-(3-cyclopentylacryloyl)-2-methoxynicotinate

To a solution of methyl 6-acetyl-2-methoxynicotinate (1.2 g, 5.74 mmol) and cyclopentanecarboxaldehyde (1.22 mL, 11.5 mmol) in methanol (30 mL) at 0° C. under nitrogen was added pyrrolidine (0.580 mL, 6.88 mmol). After 10 min, the reaction was allowed to warm up to room temperature and stirred for 2 h. The mixture was then poured into water and extracted with ethyl acetate. The organic layer was washed with brine, dried over magnesium sulfate, concentrated, and purified by silica gel column chromatography eluting with a gradient of 0%-20% ethyl acetate/heptane to obtain the title compound as a solid (0.38 g, 69%). 1H NMR (500 MHz, CHLOROFORM-d) δ ppm 1.27 (1H, t, J=7.1 Hz), 1.50 (1H, m), 1.51 (1H, dd, J=7.4, 5.0 Hz), 1.58 (3H, s), 1.67 (1H, td, J=7.6, 3.2 Hz), 1.75 (1H, m), 1.92 (1H, m), 2.77 (1H, m, J=8.1 Hz), 3.94 (3 H, s), 4.14 (2H, s), 7.23 (1H, dd, J=15.6, 8.3 Hz), 7.48 (1H, m), 7.75 (1H, d, J=7.8 Hz), 8.28 (1H, d, J=7.6 Hz).

Preparation 9: methyl 6-(3-cyclopentylacryloyl)nicotinate

The title compound was prepared by the method used for Preparation 8, Step 2 from methyl 6-acetylnicotinate (WO 2008/053300, 781 mg, 4.36 mmol) and cyclopentanecarboxaldehyde (449 mg, 4.58 mmol). 240 mg of the title compound was isolated (21%) as a solid.

Preparation 10: methyl 2-methoxy-6-(4,4,5,5-tetramethyl-1,3,2-dioxaborolan-2-yl)nicotinate
Step 1: methyl 2,6-dichloronicotinate

To a solution of 2,6-dichloronicotinic acid (130 g, 0.67 mol) in methanol (907 mL) was added sulfuric acid (18.76M, 22 g, 0.22 mol) and the mixture was refluxed for 20 h. The reaction was cooled to room temperature and poured slowly into a solution of sodium bicarbonate (42 g, 0.5 mol) in water (1 L). The mixture was concentrated to remove the methanol. The residue was diluted with ethyl acetate (1 L) and extracted with water (500 mL). The organic layer was washed with 5% aqueous sodium chloride (150 mL). The organic layer was concentrated to an oil. Heptane (150 mL) was added and the mixture was concentrated. To the residue was added heptane (580 mL) and the mixture was heated to 70° C. The solution was cooled to room temperature and stirred for 2 h. The resulting solid was collected by filtration and dried to yield the title compound as a white pale yellow solid (105 g, 75%). 1H NMR (400 MHz, DMSO-d₆) δ ppm 2.52 (1H, d, J=1.8 Hz), 3.89 (3H, s), 7.72 (1H, d, J=8.0 Hz).

Step 2: methyl 6-chloro-2-methoxynicotinate

To a solution of 2,6-dichloro-nicotinic acid methyl ester (105 g, 0.5 mol) in dichloromethane (523 mL) at −5° C. was added sodium methoxide (34.25 g, 0.63 mol) in one portion. The reaction was allowed to warm to room temperature over 4 hours. Additional sodium methoxide (5.48 g, 0.10 mol) was added and the mixture was stirred for 12 h. To the reaction was added saturated aqueous sodium bicarbonate (300 mL), water (400 mL) and dichloromethane (300 mL). The layers were separated and the aqueous layer was washed with dichloromethane (200 mL). The combined organic layers were washed with water (500 mL), dried with magnesium sulfate and filtered through celite. The filtrate was concentrated to an oil that solidified to yield the title compound (95 g, 94%). 1H NMR (500 MHz, DMSO-d₆) δ ppm 3.81 (3H, s), 3.93 (3H, s), 7.21 (1H, d, J=7.8 Hz), 8.18 (1H, d, J=7.8 Hz).

Step 3: Preparation of methyl 2-methoxy-6-(4,4,5,5-tetramethyl-1,3,2-dioxaborolan-2-yl)nicotinate

A mixture of methyl 6-chloro-2-methoxynicotinate (94 g, 0.47 mol), bis(pinacolato) diboron (130 g, 0.51 mol), potassium acetate (137 g, 1.4 mol) and 1,2-dimethoxyethane (705 mL) was sparged for 30 min with rapid nitrogen bubbling. [1,1′-bis(diphenylphosphino)ferrocene]palladium (II) chloride (19 g, 23.3 mmol) was added and the mixture was heated to reflux under nitrogen for 12 h. The mixture was diluted with ethyl acetate (150 mL) and concentrated to dryness. To the residue was added ethyl acetate (750 mL), aqueous saturated sodium bicarbonate (300 mL) and water (200 mL). The layers were separated and the organic layer was washed with water (500 mL) and concentrated. To the residue was added heptane (250 mL) and the mixture was concentrated. The residue was stirred in heptane (750 mL) at 78° C. for 15 min. The liquid was decanted and filtered through celite. To the residue was added heptane (500 mL) and the mixture was stirred at 78° C. for 30 min. The liquid was decanted and filtered through celite. The combined filtrate was stirred while cooling to room temperature while solid precipitated. The mixture was cooled to 0° C. for 30 min. The solid was filtered and dried to yield the title compound (95 g, 70%) as a tan solid. 1H NMR (400 MHz, DMSO-d₆) δ ppm 1.31 (11H, s), 2.52 (1H, m), 3.32 (1H, s), 3.81 (3 H, s), 3.93 (3H, s), 7.46 (1H, s).

Preparation 11: methyl 6-chloro-4-methoxynicotinate

To a mixture of tert-butanol (10 mL) and (trimethylsilyl)diazomethane (1.05 g, 9.16 mmol) in hexane (4.6 mL) at 0° C. under nitrogen was added 6-chloro-4-hydroxy-nicotinic acid (0.53 g, 3.05 mmol). After 5 min, the resulting mixture was warmed up to room temperature and stirred for 16 h. Additional (trimethylsilyl) diazomethane in hexanes (3.1 mL) was added and the agitation was continued for 16 h. 2.0 M (trimethylsilyl) diazomethane in ether (4.6 mL) and tert-butanol (50 mL) were added to the mixture and the agitation was continued for 16 h. The reaction mixture was concentrated and the residue was diluted with ethyl acetate, washed with sodium bicarbonate and brine, dried over magnesium sulfate, and concentrated. The residue was purified by silica gel chromatography using a gradient of 5%-30% ethyl acetate/heptane to the title compound as an off-white solid (0.324 g, 52.6%). 1H NMR (400 MHz, DMSO-d₆) δ ppm 3.81 (s, 3H) 3.95 (s, 3H) 7.37 (s, 1H) 8.56 (s, 1H).

Preparation 12: 3-methyl-6-(4,4,5,5-tetramethyl-1,3,2-dioxaborolan-2-yl)quinazolin-4(3H)-one
Step 1: 6-bromo-3-methylquinazolin-4(3H)-one

A mixture of 2-amino-5-bromobenzoic acid (250 g, 1.25 mol) and N-methylformamide (1.3 L) was refluxed in a steel bomb reactor for 6.5 h and cooled to room temperature. The mixture was poured over crushed ice, stirred for 2 h and filtered to give 6-bromo-3-methylquinazolin-4(3H)-one (180 g, 63%) as a brown solid. 1H NMR (500 MHz, METHANOL-d₄) δ ppm 3.36 (1H, m), 3.58 (3H, s), 7.58 (1H, d, J=8.5 Hz), 7.90 (1H, dd, J=8.7, 2.3 Hz), 8.30 (1H, m).

Step 2: 3-methyl-6-(4,4,5,5-tetramethyl-1,3,2-dioxaborolan-2-yl)quinazolin-4(3H)-one

A mixture of 6-bromo-3-methylquinazolin-4(3H)-one (2 g, 8.3 mmol), bis(pinacolato)diborane (2.43 g, 9.57 mmol), dichloro[1,1,'-bis(diphenylphosphino) ferrocene]palladium (II) dichloromethane adduct (0.35 g, 0.11 mmol), 1,1,′-bis(diphenylphosphino)ferrocene (0.09 g, 0.16 mmol), potassium acetate (2.5 g, 25.5 mmol) and N,N-dimethylsulfoxide (30 mL) was heated at 110° C. for 7 h under nitrogen atmosphere. The mixture was cooled to room temperature and diluted with ethyl acetate. Water (15 mL) was added and the layers were separated. The organic layer was washed 3 times with water (15 mL), washed with brine and dried over sodium sulfate. The organic layer was and concentrated to a black solid. The solid was stirred in a hexane/diethyl ether mixture to give the title compound (2.2 g, 93%) as fine grey powder. 1H NMR (400 MHz, CHLOROFORM-d) δ ppm: 8.77 (d, 1H, J=1.0 Hz), 8.11 (dd, 1H, J=8.2, 1.6 Hz), 8.04 (s, 1H), 7.64 (dd, 1H, J=8.2, 0.4 Hz), 3.57 (s, 3H), 1.34 (s, 12H).

Preparation 13: 3-oxo-3,4-dihydro-2H-benzo[b][1,4]oxazin-6-ylboronic acid
Step 1: 6-(4,4,5,5-tetramethyl-1,3,2-dioxaborolan-2-yl)-2H-benzo[b][1,4]oxazin-3(4H)-one

A mixture of 6-Bromo-4H-benzo[1,4]oxazin-3-one (100 mg, 0.439 mmol), 4,4,5,5,4′,4′,5′,5′-Octamethyl-[2,2′]bi[[1,3,2]dioxaborolanyl] (123 mg, 0.483 mmol), potassium acetate (159 mg, 1.54 mmol), 1,1-bis(diphenylphosphino)ferrocene (12.2 mg, 0.022 mmol) in dioxane (4 mL). The mixture was degassed with nitrogen for approximately 20 minutes. [1,1-bis(diphenylphosphino) ferrocene]dichloropalladium (II) (18.0 mg, 0.220 mmol) was added followed by additional 5 minutes of degassing. The mixture was heated to 100° C. for 16 h. The reaction mixture was cooled to room temperature, filtered through celite and concentrated. The residue was diluted with ethyl acetate and washed with water and brine. The organic layer was dried over sodium sulfate, filtered and concentrated. The residue was purified by silica gel column chromatography eluting with a gradient of 25%-50% ethyl acetate/heptane. The title compound was obtained as a white solid (66 mg, 55%). 1H NMR (400 MHz, METHANOL-d₄) δ ppm 1.33 (12H, s), 4.60 (2H, s), 6.93 (1H, d, J=8.3 Hz), 7.26 (1H, s), 7.36 (1H, d, J=7.9 Hz).

Step 2: 3-oxo-3,4-dihydro-2H-benzo[b][1,4]oxazin-6-ylboronic acid

A mixture of 6-(4,4,5,5-tetramethyl-1,3,2-dioxaborolan-2-yl)-2H-benzo[b][1,4]oxazin-3(4H)-one (500 mg, 1.82 mmol), polymer supported phenyl boronic acid (2200 mg, 6.4 mmol), 1M aqueous hydrochloric acid (132 mg, 3.63 mmol) in acetonitrile (12 mL) was stirred at room temperature 16 h. The reaction mixture was filtered and concentrated to yield the title compound as a solid (232 mg, 66%). 1H NMR (400 MHz, METHANOL-d₄) δ ppm 4.79 (2H, s), 6.94 (1H, d, J=8.0 Hz), 7.18 (1H, d, J=1.2 Hz), 7.27 (1H, dd, J=8.0, 1.4 Hz).

Preparation 14: 2-methyl-4-(4,4,5,5-tetramethyl-1,3,2-dioxaborolan-2-yl)benzonitrile

A mixture of dichloro[1,1,'-bis(diphenylphosphino)ferrocene]palladium (II) dichloromethane adduct (2.12 g, 2.55 mmol), potassium acetate (7.66 g, 76.5 mmol), and bis(pinacolato) diboron (7.12 g, 28.1 mmol) was flushed with nitrogen. 1,2-dimethoxyethane (130 mL) and 4-bromo-2-methylbenzonitrile (5.00 g, 25.5 mmol) were added. The reaction was stirred at 80° C. for 5 h. The reaction was cooled to room temperature and filtered through celite. The filtrate was diluted with ethyl acetate and washed with water. The organic layer was washed with brine, dried over magnesium sulfate and filtered. Silica gel was added and the mixture was concentrated. The crude material was purified by silica column chromatography eluting with a gradient of 0%-45% ethyl acetate/heptane to give the title compound (5.07 g, 82%) as a white solid. 1H NMR (500 MHz, DMSO-d₆) δ ppm 7.70 (m, 2H), 7.57 (d, 1H), 2.45 (s, 3H), 1.26 (s, 12H).

Preparation 15: 2-methyl-4-(4,4,5,5-tetramethyl-1,3,2-dioxaborolan-2-yl)benzamide
Step 1: 4-bromo-2-methylbenzamide

To a solution of 4-Bromo-2-methylbenzonitrile (3.00 g, 15.3 mmol) in ethanol (9 mL) was added 10% aqueous potassium hydroxide (8.60 mL, 15.3 mmol). The reaction was heated to 80° C. for 16 h. The reaction was cooled to 4° C. and the precipitated solid was filtered and washed with water to give the title compound as a white solid (2.45 g, 75%). 1H NMR (500 MHz, DMSO-d₆) δ ppm 7.71 (bs, 1H), 7.40 (m, 3H), 7.26 (d, 1H), 2.31 (s, 3H).

Step 2: 2-methyl-4-(4,4,5,5-tetramethyl-1,3,2-dioxaborolan-2-yl)benzamide

The title compound was prepared by the method used for Preparation 14 from 4-bromo-2-methylbenzamide (1.00 g, 4.671 mmol). 904 mg of the title compound was isolated as a tan solid (75%). 1H NMR (500 MHz, DMSO-d₆) δ ppm 7.71 (s, 1H), 7.48 (m, 2H), 7.39 (s, 1H), 7.34 (d, 2H), 2.35 (s, 3H), 1.29 (s, 12H).

Preparation 16: 2-methoxy-4-(4,4,5,5-tetramethyl-1,3,2-dioxaborolan-2-yl)benzonitrile
Step 1: 4-bromo-2-methoxybenzonitrile

A mixture of 4-bromo-2-fluorobenzonitrile (5.00 g, 25 mmol), methanol (10.0 mL, 240 mmol), and potassium carbonate (10.6 g, 75.0 mmol) in N,N-dimethylformamide (50 mL) was stirred under nitrogen at 55° C. for 16 h. The reaction was diluted with diethyl ether and water. The layers were separated. The organic layer was washed with water and brine, dried over magnesium sulfate, filtered, and concentrated to the title compound as a white solid (5.03 g, 95%). ¹H NMR (DMSO-d₆) δ ppm 7.68 (d, 1H), 7.51 (s, 1H), 7.32 (d, 1H), 3.90 (s, 3H).

Step 2: 2-methoxy-4-(4,4,5,5-tetramethyl-1,3,2-dioxaborolan-2-yl)benzonitrile

The title compound was prepared by the method used for Preparation 14 from 4-bromo-2-methoxybenzonitrile (1.00 g, 4.716 mmol). 888 mg of the title compound was isolated as a white solid (73%). 1H NMR (DMSO-d₆) δ ppm 7.73 (d, 1H), 7.35 (m, 2H), 3.94 (s, 3H), 1.31 (s, 12H).

Preparation 17: 2-methoxy-4-(4,4,5,5-tetramethyl-1,3,2-dioxaborolan-2-yl)benzamide
Step 1: 4-bromo-2-methoxybenzamide

To a solution of 4-bromo-2-methoxybenzonitrile (Preparation 16, Step 1, 2.00 g, 9.43 mmol) in ethanol (6 mL) was added 10% aqueous potassium hydroxide (5.30 mL, 943 mmol). The reaction was heated to 80° C. for 16 h. An additional amount of 10% aqueous potassium hydroxide (5.30 mL) was added to the reaction and the mixture was stirred for 8 h at 80° C. The reaction was cooled to room temperature and a solid precipitated. The mixture was filtered to yield the title compound as a white solid 4 (658 mg, 30%). ¹H NMR (500 MHz, DMSO-d₆) δ ppm 7.70 (d, 1H), 7.60 (bs, 2H), 7.54 (s, 1H), 7.22 (d, 1H), 3.90 (s, 3H).

Step 2: 2-methoxy-4-(4,4,5,5-tetramethyl-1,3,2-dioxaborolan-2-yl)benzamide

The title compound was prepared by the method used for Preparation 14 from 4-bromo-2-methoxybenzamide (600 mg, 2.61 mmol). 549 mg of the title compound was isolated as a tan solid (76%). ¹H NMR (500 MHz, DMSO-d₆) δ ppm 7.75 (d, 1H), 7.63 (bs, 1H), 7.53 (bs, 1H), 7.26 (m, 2H), 3.87 (s, 3H), 1.28 (s, 12H).

Preparation 18: methyl 2-ethoxy-4-(4,4,5,5-tetramethyl-1,3,2-dioxaborolan-2-yl)benzoate
Step 1: Methyl 2-ethoxy-4-iodobenzoate

Methyl 4-iodosalicylate (5.00 g, 18.0 mmol) was dissolved in N,N-dimethylformamide (55 mL) and cooled to 0° C. Cesium carbonate (11.7 g, 36.0 mmol) and ethyl iodide (1.91 mL, 23.9 mmol) were added. The reaction was slowly warmed to room temperature while stirring for 16 h. The reaction was diluted with ethyl acetate and washed with water. The organic layer was washed with brine and dried over magnesium sulfate, filtered, and concentrated. The residue was purified by purified by silica gel column chromatography eluting with a gradient of 0%-20% ethyl acetate/heptane to obtain the title compound as a colorless oil. (5.40 g, 98%). ¹H NMR (400 MHz, DMSO-d₆) δ ppm 7.47 (s, 1H), 7.38 (s, 2H), 4.10 (q, 2H), 3.76 (s, 3H), 1.30 (t, 3H).

Step 2: methyl 2-ethoxy-4-(4,4,5,5-tetramethyl-1,3,2-dioxaborolan-2-yl)benzoate

The title compound was prepared by the method used for Preparation 14 from methyl 2-ethoxy-4-iodobenzoate (1.90 g, 6.21 mmol). 1.45 g of the title compound was isolated as a colorless liquid (76%). 1H NMR (400 MHz, DMSO-d₆) δ ppm 7.60 (d, 1H), 7.28 (m, 2H), 4.10 (q, 2H), 3.78 (s, 3H), 1.30 (m, 15H).

Preparation 19: methyl 3-methoxy-4-(4,4,5,5-tetramethyl-1,3,2-dioxaborolan-2-yl)benzoate

1,1′-Bis(diphenylphospino)ferrocene-palladium dichloride (157 mg, 0.204 mmol), methyl 4-bromo-3-methoxybenzoate (1000 mg, 4.08 mmol), bis(pinacolato)diboron (1140 mg, 4.49 mmol), potassium acetate (843 mg, 8.16 mmol) and dioxane (3 mL) were combined in a microwave vial and bubbled with nitrogen for 5 min. The vial was sealed and heated to 100° C. in a microwave reactor for 60 min. The reaction was cooled to room temperature, filtered through celite and partitioned between ethyl acetate (10 mL) and water (10 mL). The phases were separated. The organic phase was dried over magnesium sulfate, filtered and concentrated to give an off-white solid. The solid was purified by silica gel column chromatography eluting with a gradient of 0%-50% ethyl acetate/heptane to obtain the title compound (600 mg, 50%) 1H NMR (400 MHz, DMSO-d₆) δ ppm 1.26 (s, 12H) 3.78 (s, 3H) 3.84 (s, 3H) 7.41 (d, J=1.37 Hz, 1H) 7.50 (dd, J=7.61, 1.37 Hz, 1H) 7.62 (d, J=7.61 Hz, 1H).

Preparation 20: methyl 2-(methylsulfonyl)-5-(4,4,5,5-tetramethyl-1,3,2-dioxaborolan-2-yl)benzoate
Step 1: methyl 5-bromo-2-(methylsulfonyl)benzoate

Methyl 5-bromo-2-(methylthio)benzoate (1.44 g, 5.53 mmol) was dissolved in methanol (50 mL) cooled to 0° C. To this was added a mixture of potassium peroxymonosulfate (10.4 g, 16.6 mmol) in water (50 mL). The reaction was warmed up to room temperature over 16 h. The mixture was poured into ethyl acetate and the layers were separated. The organic layer was dried over magnesium sulfate then concentrated to obtain the title compound (2.5 g, 51.4%) as a solid. 1H NMR (500 MHz, DMSO-d₆) δ ppm 3.36 (3 H, s), 3.87 (3H, s), 7.93 (1H, d, J=8.3 Hz), 8.05 (1H, m), 8.03 (1H, t, J=2.2 Hz).

Step 2: methyl 2-(methylsulfonyl)-5-(4,4,5,5-tetramethyl-1,3,2-dioxaborolan-2-yl)benzoate

To a mixture of methyl 5-bromo-2-(methylsulfonyl)benzoate (400 mg, 1.36 mmol), bis(pinacolato)diborane (416 mg, 1.64 mmol) in 1,4-dioxane (30 mL) was added 1,1′-bis(diphenylphospino)ferrocene-palladium dichloride (55.5 mg, 0.068 mmol) and potassium acetate (402 mg, 4.10 mmol). The reaction mixture was heated to 100° C. for 2 h. The reaction mixture was cooled to room temperature and filtered. The filtrate was concentrated and purified by silica gel column chromatography eluting with a gradient of 0%-100% ethyl acetate/heptane to obtain the title compound (0.200 g, 43.1%) as a solid. 1H NMR (500 MHz, CHLOROFORM-d) δ ppm 1.37 (12H, s), 3.35 (3H, s), 3.98 (3H, s), 8.09 (1H, m), 8.11 (2H, d, J=4.4 Hz).

Preparation 21: 2-methoxy-6-(4,4,5,5-tetramethyl-[1,3,2]dioxaborolan-2-yl)-nicotinamide
Step 1: 6-bromo-2-methoxy-nicotinic acid

A solution of 2,2,6,6-tetramethylpiperidine (0.766 g, 5.32 mmol) in tetrahydrofuran (5 mL) was cooled to −78° C. under nitrogen. 2.5M n-butyllithium in hexanes (2.34 mL, 0.375 g, 5.85 mmol) and the mixture was stirred at −78° C. for 30 min. To the reaction mixture was added a solution of 2-bromo-6-methoxypridine (1.00 g, 5.32 mmol) in tetrahydrofuran (5 mL) dropwise. The reaction was stirred at −78° C. for 1 h. After this time, an excess of dry ice was added to the reaction mixture and the reaction was allowed to warm to room temperature for 3 h. To the mixture was added water and ethyl acetate, the layers were separated. The aqueous layer was acidified to pH 4. The aqueous layer was extracted 3 times with ethyl acetate. The combined organic layers were washed with brine, dried over magnesium sulfate, filtered, and concentrated to an off-white solid (0.530 g, 42.9%) 1H NMR (500 MHz, DMSO-d₆) δ ppm 2.52 (2H, br. s.), 3.32 (1H, br. s.), 3.92 (1H, m), 3.90 (1H, d, J=2.9 Hz), 8.03 (1H, d, J=7.8 Hz).

Step 2: 6-bromo-2-methoxy-nicotinic acid methyl ester

To potassium carbonate (1.34 g, 9.48 mmol) in N,N-dimethylformamide (10 mL) was added 6-bromo-2-methoxy-nicotinic acid (1.10 g, 4.74 mmol) and methyl iodide (0.895 g, 6.31 mmol). The reaction was stirred for 16 h at room temperature. The reaction mixture was diluted with water and ethyl acetate, the layers were separated. The aqueous layer was washed with ethyl acetate 2 times. The combined organic layers were washed with brine and dried over magnesium sulfate, filtered and concentrated. The filtrate was concentrated and purified by silica gel column chromatography eluting with a gradient of 5%-10% ethyl acetate/heptane to obtain the title compound as a colorless oil (0.459 g, 40%). 1H NMR (500 MHz, DMSO-d₆) δ ppm 3.81 (3H, s), 3.93 (3 H, s), 7.36 (1H, d, J=7.8 Hz), 8.06 (1H, d, J=7.8 Hz).

Step 3: 6-bromo-2-methoxy-nicotinamide

6-bromo-2-methoxy-nicotinic acid methyl ester (0.45 g, 183 mmol) and ammonium hydroxide (5 mL) were combined in a sealed tube and heated to 70° C. for 3 h. The reaction was cooled to room temperature, filtered and rinsed with water to obtain the title compound as a white solid (0.278 g, 66%). 1H NMR (500 MHz, DMSO-d₆) δ ppm 3.96 (3H, s), 7.35 (1H, d, J=7.8 Hz), 7.68 (1H, br. s.), 7.76 (1H, br. s.), 8.04 (1H, d, J=7.8 Hz).

Step 4: 2-methoxy-6-(4,4,5,5-tetramethyl-[1,3,2]dioxaborolan-2-yl)-nicotinamide

The title compound was prepared by the method used for Preparation 14 from 6-bromo-2-methoxy-nicotinamide (0.270 g, 1.17 mmol). 0.325 g of the title compound isolated as a brown liquid (100%). 1H NMR (500 MHz, DMSO-d₆) δ ppm 1.16 (12H, s), 3.98 (3H, s), 7.51 (2H, br. s.), 7.63 (1H, d, J=7.3 Hz), 8.11 (1H, d, J=7.3 Hz).

Preparation 22: 6-(4,4,5,5-tetramethyl-1,3,2-dioxaborolan-2-yl)-3,4-dihydroquinolin-2(1H)-one

A mixture of the title compound was prepared by the method used for Preparation 19 from 6-bromo-3,4-dihydroquinolin-2(1H)-one (500 mg, 2.21 mmol). 316 mg of the title compound was isolated (52%) as a solid. 1H NMR (400 MHz, DMSO-d₆) δ ppm 1.27 (s, 12H) 2.44 (dd, J=8.78, 6.44 Hz, 2H) 2.87 (t, J=7.51 Hz, 2H) 6.84 (d, J=7.80 Hz, 1 H) 7.39-7.51 (m, 2H) 10.23 (s, 1H).

Preparation 23: 7-(4,4,5,5-tetramethyl-1,3,2-dioxaborolan-2-yl)-2H-benzo[b][1,4]oxazin-3(4H)-one

The title compound was prepared by the method used for Preparation 19 from 7-bromo-2H-benzo[b][1,4]oxazin-3(4H)-one (250 mg, 1.11 mmol). 291 mg of the title compound was isolated (95.6%) as a solid. 1H NMR (400 MHz, DMSO-d₆) δ ppm 1.27 (s, 12H) 4.57 (s, 2H) 6.89 (d, J=7.80 Hz, 1H) 7.13 (s, 1H) 7.26 (dd, J=7.71, 1.27 Hz, 1 H) 10.85 (s, 1H)

Preparation 24: 6-(4,4,5,5-tetramethyl-1,3,2-dioxaborolan-2-yl)-3,4-dihydroquinazolin-2(1H)-one
Step 1: 2-(aminomethyl)-4-bromoaniline

A solution of borane dimethyl sulfide complex (10 M, 10.2 ml) was added dropwise to a suspension of 2-amino-5-bromobenzonitrile (10 g, 0.102 mol) in tetrahydrofuran (400 mL) at 0° C. under nitrogen. The mixture was allowed to stir for 16 h at room temperature. After cooling to 0° C., methanol (15 mL) was added dropwise. The mixture was stirred for 30 min. Aqueous hydrochloric acid (2 M, 20 mL) was added. This resultant mixture was concentrated. The residue was triturated 3 times with diisopropyl ether (50 mL×3) to get a solid that was dried to give the title compound (4.6 g, 44.8%). 1H NMR (400 MHz, DMSO-d₆) δ ppm 7.19 (d, J=2.4 Hz, 1H), 7.05 (dd, J=8.4, 2.4 Hz, 1H), 6.55 (d, J=8.4 Hz, 1H), 5.25 (s, 2H), 3.56 (s, 2H).

Step 2: 6-bromo-3,4-dihydroquinazolin-2(1H)-one

To a solution of triphosgene (0.445 g, 1.5 mmol) in tetrahydrofuran (20 mL) was added triethylamine (0.454 g, 4.5 mmol) dropwise at 0° C. under nitrogen. After stirring for 30 min, a solution of 2-(aminomethyl)-4-bromoaniline (0.201 g, 1 mmol) in tetrahydrofuran (10 mL) was added dropwise. The mixture was allowed to stir for 16 h at room temperature. The mixture was diluted with water (15 mL) and the pH of the resultant mixture was adjusted to 8-9 by the addition of 1M aqueous sodium hydroxide. The mixture was extracted three times with ethyl acetate (30 mL). The combined organic layer was dried over sodium sulfate and concentrated. The residue was purified by re-crystallization from a mixture o dichloromethane and diethyl ether to give the title compound (0.13 g, 57.5%) as a yellow solid. 1H NMR (400 MHz, DMSO-d₆) δ ppm 9.12 (s, 1H), 7.29 (S, 1H), 7.27 (d, J=8.4 Hz, 1H), 6.87 (s, 1H), 6.70 (d, J=8.4 Hz, 1H), 4.28 (s, 2H).

Step 3: 6-4,4,5,5-tetramethyl-1,3,2-dioxaborolan-2-yl)-3,4-dihydroquinazolin-2(1H)-one

The title compound was prepared by the method used for Preparation 19 from 6-bromo-3,4-dihydroquinazolin-2(1H)-one (250 mg, 1.10 mmol). 244 mg of the title compound was isolated (80.9%) as a solid. 1H NMR (400 MHz, DMSO-d₆) δ ppm 1.26 (s, 12H) 4.32 (s, 2H) 6.74 (d, J=8.00 Hz, 1H) 6.87 (s, 1H) 7.33-7.46 (m, 2H) 9.18 (s, 1H).

Preparation 25: 4,4-dimethyl-6-(4,4,5,5-tetramethyl-1,3,2-dioxaborolane-2-yl)-1H-benzo[d][1,3]oxazin-2(4H)-one

The title compound was prepared by the method used for Preparation 19 from 6-bromo-4,4-dimethyl-1H-benzo[d][1,3]oxazin-2(4H)-one (490 mg, 1.91 mmol). 481 mg of the title compound was isolated (83%) as a solid. 1H NMR (400 MHz, DMSO-d₆) δ ppm 1.25 (s, 12H) 1.57 (s, 6H) 6.86 (d, J=7.80 Hz. 1H) 7.40-7.46 (m, 1H) 7.52 (dd, J=7.90, 1.27 Hz, 1H) 10.34 (s, 1H).

Preparation 26: 2-(4-bromo-2-methoxyphenyl)acetic acid
Step 1: Preparation of 2-(4-bromo-2-methoxyphenyl)acetonitrile

A slurry of potassium tert-butoxide (1.12 g, 9.76 mmol) in 1,2-dimethoxyethane (10 mL) was cooled to −40° C. under nitrogen. A solution of toluenesulfonylmethyl isocyanide (1.37 g, 6.98 mmol) in 1,2-dimethoxyethane (10 mL) was added dropwise over 20 min. The mixture was stirred at −40° C. for 10 min. 4-Bromo-2-methoxybenzaldehyde (1.50 g, 6.98 mmol) was added and the mixture stirred at −40° C. for 30 min. The reaction mixture was warmed to room temperature and methanol (20 mL) was added. The reaction mixture was refluxed for 1 h, cooled to room temperature and concentrated to give a brown semi-solid residue. Water (50 mL) and acetic acid were added (1.5 mL) to produce a neutral solution. Ethyl acetate (200 mL) was added and the layers were separated. The organic layer was washed with brine (10 mL), dried over magnesium sulfate, filtered and concentrated to an orange oil. The oil was purified by silica gel column chromatography eluting with a gradient of 0%-100% ethyl acetate/heptane to obtain the title compound (873 mg, 55%) as an off-white solid. 1H NMR (400 MHz, CHLOROFORM-d) δ ppm 3.57 (s, 2H) 3.80 (s, 3H) 6.97 (d, J=1.76 Hz, 1H) 7.01-7.08 (m, 1H) 7.16 (d, J=8.01 Hz, 1H).

Step 2: Preparation of 2-(4-bromo-2-methoxyphenyl)acetic acid

A mixture of 2-(4-bromo-2-methoxyphenyl)acetonitrile (873 mg, 3.86 mmol), water (5 mL), sodium hydroxide (463 mg, 11.6 mmol), and methanol (20 mL) was heated at 80° C. for 16 h. The reaction was cooled to room temperature and concentrated to give an off-white powder. The powder was suspended in water (100 mL) to give a milky solution and washed with diethyl ether (100 mL). The aqueous phase was then acidified to pH 1 with 1N aqueous hydrochloric acid and extracted with ethyl acetate (100 mL), dried over magnesium sulfate, filtered and concentrated to give of the title compound (440 mg, 47%) as a white solid. 1H NMR (400 MHz, METHANOL-d₄) δ ppm 3.52 (s, 2H) 3.66-3.78 (m, 3H) 6.94-7.11 (m, 3H).

Preparation 27: N-(6-chloropyridin-2-yl)acetamide

A mixture of 2,6-dichloropyridine (300 mg, 2.03 mmol), acetamide (145 mg, 2.43 mmol), cesium carbonate (674 mg, 2.03 mmol), 4,5-bis(diphenylphosphino)-9,9-dimethylxanthene (52.7 mg, 0.091 mmol), and tris(dibenzylideneacetone) dipalladium (63.1 mg, 0.061 mmol) in 1,4-dioxane (2.0 mL) was purged with nitrogen for 1 min. The reaction was sealed and heated to 100° C. for 16 h. The reaction mixture was cooled to room temperature filtered through celite. The filtrate was partitioned with ethyl acetate and water, separated. The organic layer was washed with brine, dried over magnesium sulfate and concentrated. The residue was purified by column chromatography using a gradient of 0-70% ethyl acetate/heptane, yielding the title compound as a white solid (224 mg, 65%). 1HNMR (DMSO-d₆, 500 MHz) δ ppm 10.77 (s, 1H), 8.04 (d, 1H), 7.82 (t, 1H), 7.18 (d, 1H), 2.08 (s, 3H).

Preparation 28: 2-chloro-6-isopropoxypyridine

Silver carbonate (1340 mg, 4.63 mmol) was added to a mixture of 6-chloropyridin-2-ol (500 mg, 0.386 mmol) and 2-bromopropane (0.435 mL, 4.63 mmol) in toluene (62 mL). The suspension was flushed with nitrogen for 2 min. The reaction vessel was sealed and heated at 80° C. for 16 h. The reaction mixture was cooled to room temperature, then filtered through celite and rinsed with ethyl acetate. The filtrate was concentrated to yield the title compound as a colorless oil (504 mg, 76%). ¹HNMR (DMSO-d₆, 500 MHz) δ ppm 7.72 (t, 1H), 7.03 (d, 1H), 6.75 (d, 1H), 5.15 (m, 1H), 1.28 (d, 6H).

Preparation 29: methyl 4-(3-cyclopentylacryloyl)-2-methoxybenzoate

Step 1; methyl 4-(1-ethoxyvinyl)-2-methoxybenzoate

A solution of methyl 4-bromo-2-methoxybenzoate (5.0 g, 20 mmol), tributyl(1-ethoxyvinyl)stannane (8.10 g, 22.4 mmol), bis(triphenylphosphine)palladium II chloride (0.438 g, 0.612 mmol), and N,N-dimethylformamide (50 mL), was stirred at 80° C. under nitrogen for 1 h. The reaction mixture was cooled to room temperature, diluted with diethyl ether (50 mL) and treated with a 10% aqueous potassium fluoride (50 mL). After stirring at room temperature for 1 h, the mixture was filtered. The solid was washed with diethyl ether. The filtrate was extracted 2 times with water (80 mL). The organic phase was dried over magnesium sulfate, filtered and concentrated. The residue was purified by silica gel column chromatography eluting with 30% ethyl acetate/heptane to yield the title compound (3.38 g, 70%) as a clear oil. 1H NMR (400 MHz, DMSO-d₆) δ ppm 1.44 (3H, t, J=7.0 Hz), 3.78 (3H, s), 3.80 (3H, s) 3.91 (2H, q), 4.30 (1H, d, J=2.9 Hz), 4.74 (1H, d, J=2.7 Hz), 7.24 (2H, m), 7.79 (1H, d, J=8.4 Hz).

Step 2: methyl 4-2-methoxybenzoate

Methyl 4-(1-ethoxyvinyl)-2-methoxybenzoate (3.38 g, 14.3 mmol) was dissolved in acetone (35.8 mL) and 1N aqueous hydrochloric acid (8.7 mL) was added. The mixture was stirred at room temperature for 1 h. The reaction mixture was diluted with water and extracted twice with diethyl ether (50 mL). The organic layers were combined and washed with aqueous saturated sodium bicarbonate. The organic phase was dried over magnesium sulfate and concentrated to the title compound (2.7 g, 91%) as a clear oil. 1H NMR (400 MHz, DMSO-d₆) δ ppm 2.63 (3H, s), 3.81 (3H, s), 3.90 (3H, s), 7.58 (s, 1H), 7.60 (1H, d), 7.78 (d, 1H).

Step 3: Methyl 4-(3-cyclopentylacryloyl)-2-methoxybenzoate

The title compound was prepared by the method used for Preparation 8, Step 2 from methyl 4-acetyl-2-methoxybenzoate (36.5 g, 175 mmol) and cyclopentanecarbaldehyde (36 mL, 337 mmol). 23.6 g of the title compound was isolated (47%) as a solid. 1H NMR (400 MHz, DMSO-d₆) δ ppm 1.44 (2H, ddd, J=8.2, 6.2, 6.0 Hz), 1.60 (2H, td, J=7.5, 3.1 Hz), 1.68 (1H, td, J=7.4, 3.2 Hz), 1.85 (1H, td, J=5.7, 4.2 Hz), 2.24 (1H, m), 2.76 (1H, m, J=8.0, 8.0, 8.0, 8.0, 8.0 Hz), 3.82 (3H, s), 3.90 (3H, s), 6.95 (1H, d, J=8.1 Hz), 6.99 (1H, d, J=8.1 Hz), 7.10 (1H, m), 7.59 (2H, m), 7.74 (1H, d, J=7.8 Hz).

Preparation 30: ethyl 2-ethoxy-6-(4,4,5,5-tetramethyl-1,3,2-dioxaborolan-2-yl)nicotinate
Step 1: ethyl 2,6-dichloronicotinate

To a solution of 2,6-dichloronicotinic acid (10 g, 52.08 mmol) in ethanol (50 mL) was added concentrated sulfuric acid (1.0 mL) and the mixture was heated to refluxed for 16 h. The reaction was concentrated. The solid residue was diluted with ethyl acetate (50 mL) and washed with water (50 mL), 1M aqueous sodium carbonate (50 mL) and saturated aqueous sodium chloride. The organic layer was dried over magnesium sulfate, filtered, and concentrated to give the title compound (7.38 g, 65%) as a light orange solid. 1H NMR (500 MHz, DMSO-d₆) δ ppm 1.32 (t, J=7.07 Hz, 3H) 4.35 (q, J=7.24 Hz, 2H) 7.72 (d, J=8.05 Hz, 1H) 8.31 (d, J=8.05 Hz, 1H).

Step 2: ethyl 6-chloro-2-ethoxynicotinate

To a solution of ethyl 2,6-dichloronicotinate (5.0 g, 22.72 mmol) in dichloromethane (25 mL) at 0° C. was added sodium ethoxide (2.12 g, 29.5 mmol) slowly. The reaction was stirred at 0° C. for 3 h and then warmed to ambient temperature over 16 h. The reaction was diluted with dichloromethane (20 mL) and water (20 mL) and the layers were separated. The aqueous layer was extracted an additional time with dichloromethane (20 mL). The organic layers were combined, washed with brine (20 mL), dried over magnesium sulfate, filtered, and concentrated to give the title compound (4.38 g, 84%) as a light yellow solid. 1H NMR (400 MHz, DMSO-d₆) δ ppm 1.22-1.40 (m, 6H) 4.27 (q, J=7.03 Hz, 2H) 4.37 (q, J=7.03 Hz, 2H) 7.18 (d, J=8.00 Hz, 1H) 8.15 (d, J=8.00 Hz, 1H).

Step 3: ethyl 2-ethoxy-6-(4,4,5,5-tetramethyl-1,3,2-dioxaborolan-2-yl)nicotinate

The title compound was prepared by the method used for Preparation 14 from ethyl 6-chloro-2-ethoxynicotinate (400 mg, 1.74 mmol). 559 mg isolated (100%). 1H NMR (500 MHz, DMSO-d₆) δ ppm 1.16 (s, 12H) 1.24-1.36 (m, 6H) 4.27 (q, J=7.07 Hz, 2H) 4.39 (q, J=7.07 Hz, 2H) 7.42 (d, J=7.32 Hz, 1H) 8.03 (d, J=7.32 Hz, 1H).

Preparation 31: methyl 2-ethoxy-6-(4,4,5,5-tetramethyl-1,3,2-dioxaborolan-2-yl)nicotinate
Step 1: methyl 6-chloro-2-ethoxynicotinate

To a solution of methyl 2,6-dichloronicotinate (preparation 10, step 1, 5.0 g, 24.3 mmol) in dichloromethane (25 mL) at 0° C. was added sodium ethoxide (2.26 g, 31.6 mmol) slowly. The reaction was stirred at 0° C. for 3 h. The reaction was diluted with water (25 mL) and the phases were separated. The organic layer was washed with saturated aqueous sodium chloride (10 mL) then dried over magnesium sulfate, filtered and concentrated to give a residue. The residue was purified by silica gel column chromatography eluting a gradient of 0%-5% ethyl acetate/heptane to yield the title compound (2.55 g, 49%) as a colorless oil. 1H NMR (400 MHz, DMSO-d₆) δ ppm 1.29 (t, J=7.13 Hz, 3H) 3.93 (s, 3H) 4.27 (q, J=7.09 Hz, 2H) 7.21 (d, J=7.81 Hz, 1H) 8.17 (d, J=8.00 Hz, 1H).

Step 2: methyl 2-ethoxy-6-(3,3,4,4-tetramethylcyclopentyl)nicotinate

The title compound was prepared by the method used for Preparation 14 from methyl 6-chloro-2-ethoxynicotinate (2.50 g, 9.3 mmol). 2.8 g isolated (100%). 1H NMR (500 MHz, DMSO-d₆) δ ppm 1.25-1.38 (m, 14H) 3.93 (s, 3H) 4.27 (q, J=7.07 Hz, 3H) 7.45 (d, J=7.56 Hz, 1H) 8.04 (d, J=7.32 Hz, 1H).

Preparation 32: 4-(3-chloro-5-(3,3-difluorocyclobutyl)-4,5-dihydro-1H-pyrazol-1-yl)-2-methylbenzonitrile
Step 1: ethyl 3-(3,3-difluorocyclobutyl)acrylate

To a solution of ethyl 3,3-difluorocyclobutanecarboxylate (1.94 g, 11.82 mmol) in dichloromethane (40 mL) at −78° C. was added diisobutyl aluminum hydride (13 mL of a 1.0M solution in hexanes, 13.0 mmol). The reaction was stirred at −78° C. for 45 min. Saturated aqueous ammonium chloride (40 mL) was added, and the resulting mixture stirred overnight at room temperature. The biphasic mixture was filtered through celite. The organic layer was dried over sodium sulfate, filtered and concentrated to give 3,3-difluorocyclobutanecarboxaldehyde which was used immediately without further purification.

To a suspension of sodium hydride, 60 wt % dispersion in mineral oil (487 mg, 12 mmol) in tetrahydrofuran at 0° C. was added triethyl phosphonoacetate (2.4 mL, 12.0 mmol) dropwise. After addition was complete, the mixture was allowed to warm to room temperature, and stirred until the suspension cleared (˜10 min.). 3,3-difluorocyclobutanecarboxaldehyde was added as a solution in tetrahydrofuran (10 mL). The resulting solution was stirred at room temperature 4 h. Diethyl ether (40 mL) was added, and the reaction was quenched by addition of saturated aqueous ammonium chloride (20 mL). The organic layer was dried over magnesium sulfate, filtered and concentrated. The crude residue was purified by silica gel chromatography eluting with a gradient of 0%-5% ethyl acetate in heptane to yield ethyl 3-(3,3-difluorocyclobutyl)acrylate (498 mg, 22%) as a yellow oil. 1H NMR (400 MHz, CHLOROFORM-d) δ ppm 1.30 (3H, t, J=7.1 Hz), 2.49 (2H, m), 2.83 (2H, m), 2.94 (1 H, m), 4.21 (1H, q, J=7.2 Hz), 5.85 (1H, dd, J=15.5, 1.3 Hz), 7.00 (1H, dd, J=15.5, 7.3 Hz).

Step 2: 4-(5-(3,3-difluorocyclobutyl)-3-oxopyrazolidin-1-yl)-2-methylbenzonitrile

To a solution of ethyl 3-(3,3-difluorocyclobutyl)acrylate (500 mg, 2.63 mmol) and 4-hydazinyl-2-methylbenzonitrile (471 mg, 3.20 mmol) in ethanol (8 mL) was added sodium ethoxide (2.0 mL of a 21 wt % solution in EtOH, 5.4 mmol) The mixture was stirred at reflux 1 h. The reaction was cooled to room temperature and diluted with water (20 mL). 1M aqueous hydrochloric acid was added to adjust the pH of the mixture to about 2 and the mixture extracted with ethyl acetate. The organic layer was dried over magnesium sulfate, filtered and concentrated to provide a red solid. The crude solid was purified by silica gel chromatography eluting with a gradient of 50%-100% ethyl acetate in heptane to yield 4-(5-(3,3-difluorocyclobutyl)-3-oxopyrazolidin-1-yl)-2-methylbenzonitrile (174 mg, 23%). 1H NMR (400 MHz, DMSO-d₆) δ ppm 1.98 (1H, d, J=16.8 Hz), 2.44 (3H, s), 2.41 (2H, m) 2.52 (1H, m), 2.67 (2H, m), 2.86 (1H, dd, J=16.8, 8.4 Hz), 4.28 (1H, t, J=8Hz), 6.92 (1H, dd, J=2.1, 8.6 Hz), 7.01 (1H, d, J=2.1 Hz), 7.65 (1H, d, J=8.6 Hz), 10.36 (1H, s).

Step 3: 4-(3-chloro-5-(3,3-difluorocyclobutyl)-4,5-dihydro-1H-pyrazol-1-yl)-2-methylbenzonitrile

The title compound was prepared by the method used for Preparation 2, Step 2 from 4-(5-(3,3-difluorocyclobutyl)-3-oxopyrazolidin-1-yl)-2-methylbenzonitrile (174 mg, 0.60 mmol). 131 mg isolated (71%) as a red oil. 1H NMR (400 MHz, CHLOROFORM-d) δ ppm 2.36 (2H, m), 2.50 (3H, s), 2.67 (2H, m), 2.87 (1H, dd, J=17.9, 4.1 Hz), 4.60 (1 H, m), 6.79 (1H, dd, J=8.6, 2.3 Hz), 6.95 (1H, d, J=2.3 Hz), 7.47 (1H, d, J=8.8 Hz).

### Example 1

### Methyl 6-(1-(4-cyano-3-methylphenyl)-5-cyclopentyl-4,5-dihydro-1H-pyrazol-3-yl)-2-methoxynicotinate

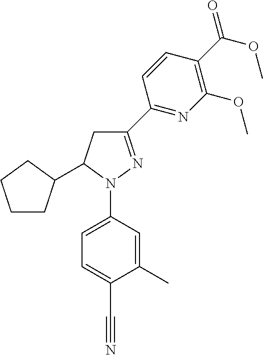

Method 1: To a solution of 4-(3-chloro-5-cyclopentyl-4,5-dihydro-1H-pyrazol-1-yl)-2-methylbenzonitrile (Preparation 2, 83 g, 288 mmol) and methyl 2-methoxy-6-(4,4,5,5-tetramethyl-1,3,2-dioxaborolan-2-yl)nicotinate (Preparation 10, 89 g, 303 mmol) in 1,2-dimethoxyethane (1.25 L) was added 1M sodium carbonate (723 mL, 692 mmol) The reaction mixture was sparged for 30 min with rapid nitrogen bubbling. Tetrakis(triphenyl phosphine)palladium (10.1 g, 8.65 mmol) was added and the reaction mixture was heated to 80° C. under nitrogen for 12 h. The reaction mixture was cooled to room temperature and concentrated. Ethyl acetate (500 mL) was added to the residue and the reaction was concentrated. To the residue was added ethyl acetate (2 L) and 5% aqueous sodium carbonate (1 L) and the mixture was heated to 50° C. The layers were separated and the organic layer was washed with brine (500 mL). The organic layer was concentrated and the residue was purified by silica gel column chromatography eluting with a gradient of 15%-70% ethyl acetate in heptane to give a yellow solid. To the solid was added heptane (500 mL) and the mixture was filtered to yield a yellow solid that was dried under vacuum for 3 h to yield the title compound (82 g, 68%). 1H NMR (500 MHz, DMSO-d₆) δ ppm 1.06 (1H, m), 1.30 (1H, dd, J=8.4, 3.0 Hz), 1.40 (1H, m), 1.49 (2H, m), 1.51 (1H, d, J=8.3 Hz), 1.77 (1H, dd, J=11.8, 4.3 Hz), 2.44 (3H, s), 2.53 (1H, m), 3.22 (1H, dd, J=18.5, 4.1 Hz), 3.33 (1H, m), 3.48 (1H, dd, J=18.3, 11.7 Hz), 3.82 (3H, s), 3.99 (3H, s), 4.91 (1H, dt, J=11.7, 4.1 Hz), 7.14 (1H, dd, J=8.5, 2.2 Hz), 7.25 (1H, d, J=1.7 Hz), 7.60 (1H, d, J=8.8 Hz), 7.71 (1H, d, J=8.8 Hz), 8.17 (1H, d, J=8.8 Hz).

### Example 2

### ((R)-methyl 6-(1-(4-cyano-3-methylphenyl)-5-cyclopentyl-4,5-dihydro-1H-pyrazol-3-yl)-2-methoxynicotinate

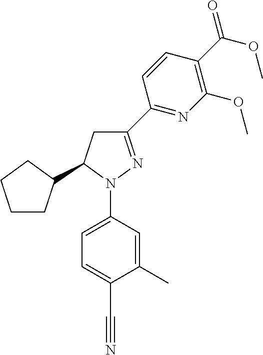

Method 1: The title compound was prepared from methyl 6-(1-(4-cyano-3-methylphenyl)-5-cyclopentyl-4,5-dihydro-1H-pyrazol-3-yl)-2-methoxynicotinate (Example 1) using chiral SFC. Column: AD-H, 21×250, Mobile phase: 65/35 carbon dioxide/methanol, 65 mL/min. First eluting peak: chiral SFC t_{R}=6.45 min (Chiralpak AD-H 4.6 mm×25 cm; 63/35 carbon dioxide/methanol). 1H NMR (500 MHz, DMSO-d₆) δ ppm 1.08 (1H, m), 1.29 (1H, br. s.), 1.40 (1H, m), 1.50 (1H, m), 1.51 (1H, d, J=6.8 Hz), 1.76 (1H, br. s.), 2.45 (3H, s), 2.52 (1H, br. s.), 3.22 (1H, dd, J=18.3, 4.1 Hz), 3.32 (3 H, s), 3.48 (1H, dd, J=18.4, 11.8 Hz), 3.82 (3H, s), 3.99 (3H, s), 4.91 (1H, dt, J=11.7, 4.1 Hz), 7.15 (1H, dd, J=8.5, 2.2 Hz), 7.26 (1H, d, J=1.7 Hz), 7.60 (1H, d, J=8.8 Hz), 7.71 (1H, d, J=8.1 Hz), 8.17 (1H, d, J=7.8 Hz).

Method 2: (R)-4-(3-chloro-5-cyclopentyl-4,5-dihydro-1H-pyrazol-1-yl)-2-methylbenzonitrile (Preparation 3, 595 mg, 2.07 mmol) and methyl 2-methoxy-6-(4,4,5,5-tetramethyl-1,3,2-dioxaborolan-2-yl)nicotinate (Preparation 10, 727 mg, 2.48 mmol) were combined in dimethoxyethane (12 mL) and 2M aqueous sodium carbonate (2.27 mL, 484 mg, 4.55 mmol) was added followed by tetrakis(triphenylphosphine)palladium (119 mg, 0.103 mmol) and the mixture was heated at 80° C. for 16 h. The reaction was cooled to room temperature, filtered through celite. To the filtrate was added ethyl acetate and water, the layers were separated. The organic layer was washed with brine, dried over magnesium sulfate, filtered and concentrated. The residue was purified by silica gel column chromatography eluting with a gradient of 0%-40% ethyl acetate/heptane to give the title compound (484 mg, 56%) as a yellow solid. 1H NMR (400 MHz, DMSO-d₆) δ ppm 1.09 (2H, t, J=6.9 Hz), 1.30 (1H, br. s.), 1.51 (2H, d, J=8.6 Hz), 1.76 (1H, br. s.), 2.45 (3H, s), 3.22 (1H, dd, J=18.5, 4.1 Hz), 3.31 (2H, s), 3.38 (1H, q, J=7.0 Hz), 3.48 (1H, dd, J=18.4, 11.8 Hz), 3.82 (3H, s), 3.99 (3H, s), 4.91 (1H, d, J=11.7 Hz), 7.15 (1H, dd, J=8.7, 2.0 Hz), 7.25 (1H, s), 7.60 (1H, d, J=8.8 Hz), 7.71 (1H, d, J=8.0 Hz), 8.17 (1H, d, J=8.0 Hz).

Method 3: (R)-4-(3-chloro-5-cyclopentyl-4,5-dihydro-1H-pyrazol-1-yl)-2-methylbenzonitrile (Preparation 3, 14.42 g, 50.11 mmol) and methyl 2-methoxy-6-(4,4,5,5-tetramethyl-1,3,2-dioxaborolan-2-yl)nicotinate (Preparation 10, 14.72 g, 50.22 mmol) were combined in tetrahydrofuran (150 mL) and 1M aqueous potassium carbonate (150 mL) and the mixture was purged with nitrogen for 15 min. Bis(triphenylphosphine)palladium(II) chloride (380 mg, 0.54 mmol) was added and the mixture was heated to reflux for 3.5 h. The reaction was cooled to room temperature. Ethyl acetate (150 mL) was added, the mixture was stirred and the layers were separated. The organic layer was washed with brine (100 mL) and filtered through celite. The filtrate was dried over magnesium sulfate filtered and concentrated. To the residue was added methyl t-butyl ether (200 mL). The mixture was stirred for 15 minutes and heptanes (200 mL) was added. The mixture was stirred at RT for 18 h. The mixture was filtered and the solids collected were rinsed with 50% methyl t-butyl ether in heptanes. The solid was dried under vacuum at 40° C. to give the title compound (16.29 g, 78%) as a yellow solid.

### Example 3

### 6-[1-(4-cyano-3-methyl-phenyl)-5-cyclopentyl-4,5-dihydro-1H-pyrazol-3-yl]-2-methoxy-nicotinic acid

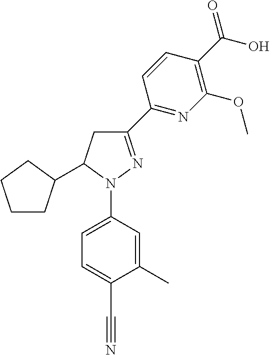

To a solution of methyl 6-(3-cyclopentylacryloyl)-2-methoxynicotinate (Preparation 8, 0.390 g, 1.35 mmol) and 4-hydrazino-2-methyl-benzonitrile (WO 2008/053300, 0.347 g, 1.89 mmol) in ethanol (15 mL) bubbled with nitrogen was added a solution of 21% sodium ethoxide in ethanol (1.51 mL, 4.04 mmol). The mixture was heated to 80° C. for 3 h. The mixture was cooled to room temperature, poured into a diluted hydrochloride acid solution, and extracted with ethyl acetate. The organic phase was washed with brine, dried over magnesium sulfate, and concentrated. The residue was purified by silica gel column chromatography eluting with a gradient of 0-20% methanol in dichloromethane to obtain the title compound (0.300 g, 55%) as a dark yellow solid. ¹H NMR (500 MHz, CHLOROFORM-d) δ ppm 0.89 (1H, s), 1.27 (4H, t, J=7.2 Hz), 1.57 (5 H, br. s.), 2.54 (3H, s), 3.27 (1H, m), 3.44 (1H, dd, J=18.2, 12.1 Hz), 4.13 (1H, q, J=7.2 Hz), 4.24 (3H, s), 7.03 (1H, dd, J=8.5, 2.2 Hz), 7.15 (1H, d, J=8.5 Hz), 7.50 (1 H, d, J=8.2 Hz), 7.87 (1H, d, J=8.1 Hz), 8.44 (1H, d, J=8.1 Hz).

### Example 4

### (R)-6-(1-(4-cyano-3-methylphenyl)-5-cyclopentyl-4,5-dihydro-1H-pyrazol-3-yl)-2-methoxynicotinic acid

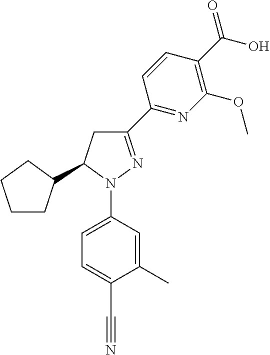

Method 1: The title compound was prepared from 6-[1-(4-Cyano-3-methyl-phenyl)-5-cyclopentyl-4,5-dihydro-1H-pyrazol-3-yl]-2-methoxy-nicotinic acid (Example 3) using chiral SFC. Column: AD-H, 30×250 mm, 50/50 carbon dioxide/methanol. Second eluting peak: chiral SFC t_{R}=4.810 min (Chiralcel AS-H, 75/25 carbon dioxide/methanol). The methanol solution containing the desired enantiomer was concentrated to dryness to yield a yellow solid. 1H NMR (500 MHz, CHLOROFORM-d) δ ppm 0.89 (1

H, s), 1.27 (4H, t, J=7.2 Hz), 1.57 (5H, br. s.), 2.54 (3H, s), 3.27 (1H, m), 3.44 (1H, dd, J=18.2, 12.1 Hz), 4.13 (1H, q, J=7.2 Hz), 4.24 (3H, s), 7.03 (1H, dd, J=8.5, 2.2 Hz), 7.15 (1H, d, J=8.5 Hz), 7.50 (1H, d, J=8.2 Hz), 7.87 (1H, d, J=8.1 Hz), 8.44 (1H, d, J=8.1 Hz).

Method 2: ((R)-methyl 6-(1-(4-cyano-3-methylphenyl)-5-cyclopentyl-4,5-dihydro-1H-pyrazol-3-yl)-2-methoxynicotinate (Example 2, 33.5 g, 80 mmol) was dissolved in tetrahydrofuran (330 mL) and to this was added a solution of lithium hydroxide (2.9 g, 122 mmol) in water (60 mL). The reaction mixture was stirred at 40° C. for 18 h. The reaction was cooled to room temperature and 1N aqueous hydrochloric acid (122 mL) was added (pH=1.8). The mixture was stirred for 1 h and phase separated. The aqueous layer was washed with 2-methyltetrahydrofuran (100 mL). The combined organic layers were washed with brine (100 mL), dried over magnesium sulfate, filtered through celite and concentrated. Methyl tert-butyl ether (300 mL) was added and the suspension was stirred for 2 h. The mixture was filtered and the yellow solid was dried under vacuum at 35° C. to yield the title compound (19.7 g, 61%). The mother liquor was concentrated and methyl tert-butyl ether (130 mL) and heptane (130 mL) was added. The slurry was heated to reflux and cooled to room temperature for 4 h. The solid was filtered and dried to yield additional title compound (10.6 g, 33%). 1H NMR (400 MHz, CHLOROFORM-d) δ ppm 0.88 (1H, m), 1.13 (2H, m), 1.28 (3H, m), 1.48 (2H, m), 1.62 (3H, m), 1.82 (1H, m, J=11.9, 8.0, 3.9, 3.9 Hz), 2.54 (3H, s), 2.62 (1H, m), 2.62 (1H, d, J=3.7 Hz), 3.27 (1H, d, J=4.6 Hz), 3.23 (1H, m), 3.42 (1H, d, J=12.0 Hz), 4.24 (3H, s), 4.77 (1H, ddd, J=11.8, 4.4, 4.1 Hz), 7.03 (1H, dd, J=8.7, 2.1 Hz), 7.15 (1H, d, J=1.7 Hz), 7.50 (1H, d, J=8.3 Hz), 7.87 (1H, d, J=8.3 Hz), 8.44 (1H, d, J=7.9 Hz).

Method 3: ((R)-methyl 6-(1-(4-cyano-3-methylphenyl)-5-cyclopentyl-4,5-dihydro-1H-pyrazol-3-yl)-2-methoxynicotinate (Example 2, 175 mg, 7.17 mmol) was dissolved in tetrahydrofuran (15 mL) and to this was added 2M aqueous lithium hydroxide (3.58 mL, 7.17 mmol). The reaction mixture was stirred at 40° C. for 18 h. The reaction was cooled to room temperature and 1 M aqueous hydrochloric acid was added (pH=4). The mixture was extracted three times with methylene chloride. The combined organic layers were washed with brine, dried over magnesium sulfate, filtered and concentrated to dryness. To the residue was added diethyl ether and the mixture was sonicated. The resulting suspension was filtered to yield the title compound (1.83 g, 95%).

1H NMR (500 MHz, DMSO-d₆) δ ppm 1.09 (2H, t, J=7.1 Hz), 1.31 (1H, dd, J=8.1, 3.9 Hz), 1.40 (1H, d, J=4.1 Hz), 1.50 (2H, m), 1.58 (1H, br. s.), 1.77 (1H, ddd, J=7.8, 4.3, 4.0 Hz), 2.44 (3H, s), 2.54 (2H, m), 2.52 (3H, d, J=2.2 Hz), 3.22 (1H, dd, J=18.5, 4.1 Hz), 3.32 (1H, s), 3.38 (1H, q, J=7.1 Hz), 3.48 (1H, dd, J=18.3, 11.7 Hz), 3.98 (3H, s), 4.90 (1H, dt, J=11.9, 4.1 Hz), 7.14 (1H, dd, J=8.7, 2.1 Hz), 7.25 (1H, d, J=1.7 Hz), 7.59 (1H, d, J=8.8 Hz), 7.69 (1H, d, J=7.8 Hz), 8.15 (1H, d, J=7.8 Hz)

Method 4: ((R)-methyl 6-(1-(4-cyano-3-methylphenyl)-5-cyclopentyl-4,5-dihydro-1H-pyrazol-3-yl)-2-methoxynicotinate (Example 2, 16 g, 38.2 mmol) was dissolved in tetrahydrofuran (160 mL) and to this was added 2.6% aqueous sodium hydroxide (57 mL). The reaction mixture was stirred at room temperature for 18 h. To the reaction was added 1 N aqueous hydrochloric acid (60 mL) was added (pH=1.2). To the mixture was added ethyl acetate (160 mL) and the mixture was phase separated. The organic layer was washed with brine (100 mL), dried over magnesium sulfate and concentrated. Isopropyl alcohol (155 mL) was added and the mixture was heated to reflux. The mixture was cooled to room temperature. The mixture was filtered and the yellow solid was dried under vacuum to yield the title compound (11.86 g, 77%).

1H NMR (400 MHz, DMSO-d₆) δ ppm 1.03 (2H, d, J=6.2 Hz), 1.28 (2H, m), 1.31 (1H, d, J=8.3 Hz), 1.39 (1H, d, J=3.3 Hz), 1.51 (2H, m), 1.58 (1H, br. s.), 1.77 (1H, dd, J=11.8, 3.9 Hz), 2.44 (3H, s), 2.52 (3H, d, J=3.7 Hz), 3.22 (1H, dd, J=18.5, 3.9 Hz), 3.32 (1H, s), 3.45 (1H, d, J=12.0 Hz), 3.98 (3H, s), 4.89 (1H, dt, J=11.6, 4.1 Hz), 7.13 (1H, dd, J=8.7, 2.1 Hz), 7.24 (1H, s), 7.59 (1H, d, J=8.7 Hz), 7.68 (1H, d, J=7.9 Hz), 8.15 (1H, d, J=7.9 Hz), 12.94 (1H, s)

Powder X-ray Diffraction Analysis (PXRD): The powder X-ray diffraction patterns of (R)-6-(1-(4-cyano-3-methylphenyl)-5-cyclopentyl-4,5-dihydro-1H-pyrazol-3-yl)-2-methoxynicotinic acid were carried out on a Bruker D5000 diffractometer using copper radiation (wavelength: 1.54056 Å). The tube voltage and amperage were set to 40 kV and 40 mA, respectively. The divergence and scattering slits were set at 1 mm, and the receiving slit was set at 0.6 mm. Diffracted radiation was detected by a Kevex PSI detector. A theta-two theta continuous scan at 2.4°/min (1 sec/0.04° step) from 3.0 to 40° 2θ was used. An alumina standard was analyzed to check the instrument alignment. Data were collected and analyzed using Bruker axis software Version 7.0. Samples were prepared by placing them in a quartz holder. It should be noted that Bruker Instruments purchased Siemans; thus, Bruker D5000 instrument is essentially the same as a Siemans D5000. Eva Application 13.0.0.3 software was used to visualize and evaluate PXRD spectra. PXRD data files (.raw) were not processed prior to peak searching. Generally, a Threshold value of 2 and a Width value of 0.3 were used to make preliminary peak assignments. The output of automated assignments was visually checked to ensure validity and adjustments manually made if necessary. These peak values for each form are summarized in tables below.

To perform an X-ray diffraction measurement on a Bragg-Brentano instrument like the Bruker system used for measurements reported herein, the sample is typically placed into a holder which has a cavity. The sample powder is pressed by a glass slide or equivalent to ensure a random surface and proper sample height. The sample holder is then placed into the instrument. The incident X-ray beam is directed at the sample, initially at a small angle relative to the plane of the holder, and then moved through an arc that continuously increases the angle between the incident beam and the plane of the holder. Measurement differences associated with such X-ray powder analyses result from a variety of factors including: (a) errors in sample preparation (e.g., sample height), (b) instrument errors (e.g. flat sample errors), (c) calibration errors, (d) operator errors (including those errors present when determining the peak locations), and (e) the nature of the material (e.g. preferred orientation and transparency errors). Calibration errors and sample height errors often result in a shift of all the peaks in the same direction. Small differences in sample height when using a flat holder will lead to large displacements in XRPD peak positions. A systematic study showed that, using a Shimadzu XRD-6000 in the typical Bragg-Brentano configuration, sample height difference of 1 mm lead to peak shifts as high as 1°2θ (Chen et al.; J Pharmaceutical and Biomedical Analysis, 2001; 26, 63). These shifts can be identified from the X-ray Diffractogram and can be eliminated by compensating for the shift (applying a systematic correction factor to all peak position values) or recalibrating the instrument. As mentioned above, it is possible to rectify measurements from the various machines by applying a systematic correction factor to bring the peak positions into agreement. In general, this correction factor will bring the measured peak positions from the Bruker into agreement with the expected peak positions and may be in the range of 0 to 0.2° 2θ.

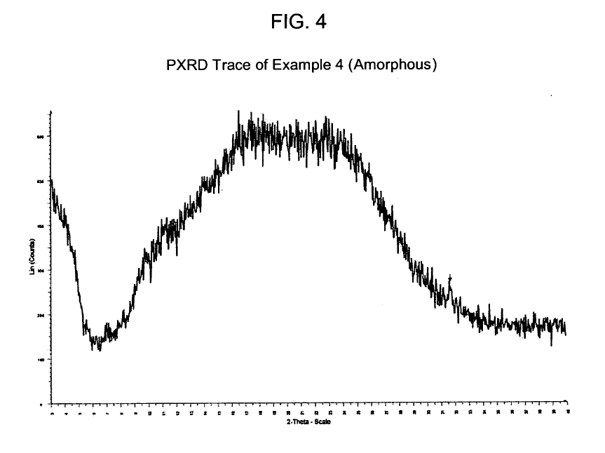

(R)-6-(1-(4-cyano-3-methylphenyl)-5-cyclopentyl-4,5-dihydro-1H-pyrazol-3-yl)-2-methoxynicotinic acid, amorphous: Analysis of the solid obtained from Method 1 by PXRD (See FIG. 4) indicated that this material was not crystalline.

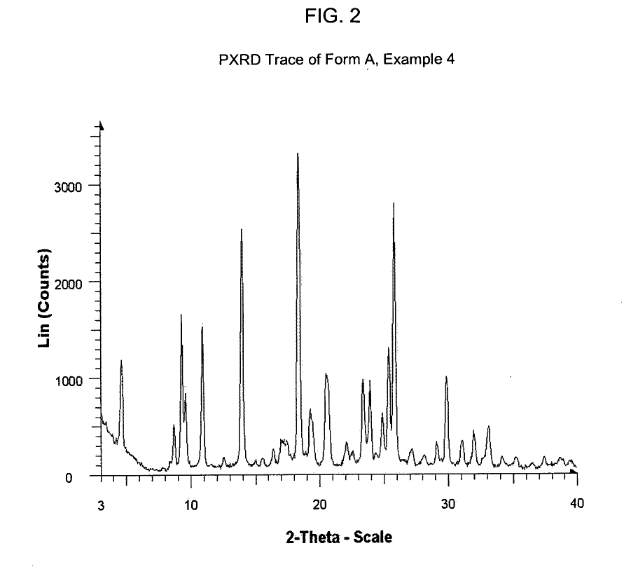

(R)-6-(1-(4-cyano-3-methylphenyl)-5-cyclopentyl-4,5-dihydro-1H-pyrazol-3-yl)-2-methoxynicotinic acid, Form A: The title compound obtained from Method 2 was determined to be the methyl tert-butyl ether solvate; the title compound obtained from Method 3 was determined to be the diethyl ether solvate and the title compound obtained from Method 4 was determined to be the isopropyl alcohol solvate. All of these samples were determined to consist of the same powder X-ray pattern and designated Form A. Crystalline Form A is characterized by the following powder x-ray diffraction pattern, provided in FIG. 2, expressed in terms of the degree 2θ and relative intensities with a relative intensity of ≧4.7% measured on a Bruker D5000 diffractometer with CuKα radiation:

| Angle | Relative Intensity* |
|---|---|
| (Degree 2θ) | (≧4.7%) |
| 4.6 | 35.7 |
| 8.7 | 15.5 |
| 9.3 | 49.8 |
| 9.6 | 25.0 |
| 10.9 | 46.1 |
| 12.5 | 5.3 |
| 14.0 | 76.5 |
| 15.0 | 4.7 |
| 15.5 | 4.9 |
| 16.4 | 7.9 |
| 18.4 | 100.0 |
| 19.3 | 20.3 |
| 20.6 | 31.1 |
| 22.1 | 10.0 |
| 22.6 | 7.3 |
| 23.4 | 29.4 |
| 24.0 | 29.0 |
| 24.4 | 6.5 |
| 24.9 | 18.8 |
| 25.4 | 39.3 |
| 25.9 | 84.4 |
| 27.2 | 7.7 |
| 28.1 | 5.8 |
| 29.1 | 9.9 |
| 29.9 | 30.1 |
| 31.1 | 10.2 |
| 32.0 | 13.3 |
| 33.1 | 14.8 |
| 34.2 | 5.2 |
| 35.3 | 5.1 |
| 37.4 | 5.1 |

*The relative intensities may change depending on the crystal size and morphology.

Conversion of (R)-6-(1-(4-cyano-3-methylphenyl)-5-cyclopentyl-4,5-dihydro-1H-pyrazol-3-yl)-2-methoxynicotinic acid, Form A to Form B

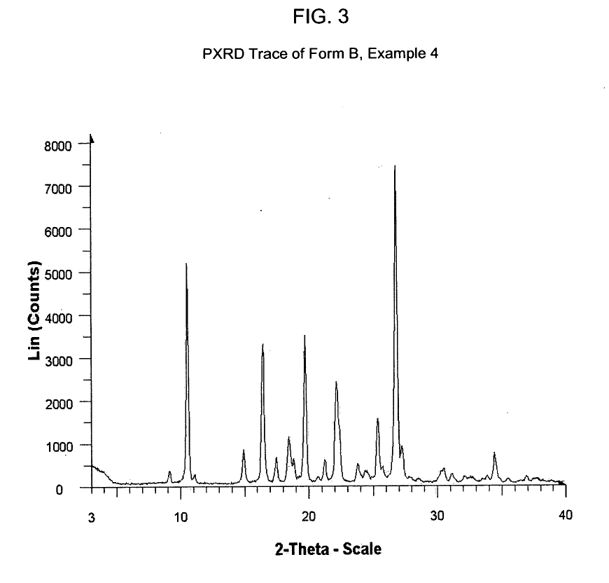

Method 5: A suspension of (R)-6-(1-(4-cyano-3-methylphenyl)-5-cyclopentyl-4,5-dihydro-1H-pyrazol-3-yl)-2-methoxynicotinic acid, methyl tert-butyl ether solvate Form A (Method 2, 500 mg) in water (10 mL) was heated to reflux for 30 min. The mixture was cooled to room temperature and stirred for 48 h. The mixture was filtered and the solid was dried to obtain anhydrous (R)-6-(1-(4-cyano-3-methylphenyl)-5-cyclopentyl-4,5-dihydro-1H-pyrazol-3-yl)-2-methoxynicotinic acid (465 mg, 93%). 1H NMR (400 MHz, DMSO-d₆) δ ppm 1.05 (1H, d, J=9.6 Hz), 1.27 (3H, d, J=11.1 Hz), 1.50 (2H, m), 1.50 (2H, d, J=8.4 Hz), 1.57 (1H, br. s.), 1.75 (1H, d, J=3.9 Hz), 2.44 (4H, s), 2.51 (1H, br. s.), 3.21 (1H, dd, J=18.5, 4.2 Hz), 3.49 (1H, d, J=11.9 Hz), 3.45 (1H, d, J=11.5 Hz), 3.97 (4H, s), 4.89 (1H, dt, J=11.7, 4.0 Hz), 7.13 (1H, dd, J=8.6, 2.1 Hz), 7.24 (1H, d, J=1.8 Hz), 7.59 (1H, d, J=8.6 Hz), 7.68 (1H, d, J=7.8 Hz), 8.14 (1H, d, J=7.8 Hz), 12.97 (1H, br. s.) This material was determined to be anhydrous Form B (See FIG. 3). Method 6: A suspension of (R)-6-(1-(4-cyano-3-methylphenyl)-5-cyclopentyl-4,5-dihydro-1H-pyrazol-3-yl)-2-methoxynicotinic acid, isopropyl alcohol solvate Form A (Method 4, 11.58 g) in 50% ethanol/water (200 mL) was heated to 80° C. for 2.5 h. The mixture was cooled to room temperature. The mixture was filtered and the solid was dried under vacuum at 40° C. to obtain anhydrous (R)-6-(1-(4-cyano-3-methylphenyl)-5-cyclopentyl-4,5-dihydro-1H-pyrazol-3-yl)-2-methoxynicotinic acid (465 mg, 93%). ¹H NMR (400 MHz, DMSO-d₆) δ ppm 1.05 (1H, d, J=9.6 Hz), 1.27 (3H, d, J=11.1 Hz), 1.50 (2H, m), 1.50 (2H, d, J=8.4 Hz), 1.57 (1H, br. s.), 1.75 (1H, d, J=3.9 Hz), 2.44 (4 H, s), 2.51 (1H, br. s.), 3.21 (1H, dd, J=18.5, 4.2 Hz), 3.49 (1H, d, J=11.9 Hz), 3.45 (1H, d, J=11.5 Hz), 3.97 (4H, s), 4.89 (1H, dt, J=11.7, 4.0 Hz), 7.13 (1H, dd, J=8.6, 2.1 Hz), 7.24 (1H, d, J=1.8 Hz), 7.59 (1H, d, J=8.6 Hz), 7.68 (1H, d, J=7.8 Hz), 8.14 (1H, d, J=7.8 Hz), 12.97 (1H, br. s.) This material was determined to be anhydrous Form B (See FIG. 3).

Crystalline Form B is characterized by the following powder x-ray diffraction pattern, provided in FIG. 3, expressed in terms of the degree 2θ and relative intensities with a relative intensity of ≧3.5% measured on a Bruker D5000 diffractometer with CuKα radiation:

| Angle | Relative Intensity* |
|---|---|
| (Degree 2θ) | (≧3.5%) |
| 9.2 | 4.6 |
| 10.6 | 69.6 |
| 11.1 | 3.7 |
| 14.9 | 11.4 |
| 16.4 | 44.4 |
| 17.5 | 8.9 |
| 18.5 | 15.3 |
| 18.8 | 8.4 |
| 19.7 | 47.1 |
| 21.2 | 8.0 |
| 22.2 | 32.5 |
| 22.4 | 19.8 |
| 23.8 | 6.9 |
| 24.4 | 4.5 |
| 25.4 | 21.0 |
| 25.8 | 6.0 |
| 26.8 | 100.0 |
| 27.3 | 12.3 |
| 30.5 | 5.2 |
| 31.2 | 3.5 |
| 34.5 | 10.3 |

*The relative intensities may change depending on the crystal size and morphology.

Characteristic 2θ peaks for solid forms of (R)-6-(1-(4-cyano-3-methylphenyl)-5-cyclopentyl-4,5-dihydro-1H-pyrazol-3-yl)-2-methoxynicotinic acid

| | |
|---|---|
| Form | Angle (Degree 2θ) |

| | | | | |
|---|---|---|---|---|
| A | 4.6 | 10.9 | 14.0 | 18.4 |
| B | 9.2 | 10.6 | 19.7 | 26.8 |

### Example 5

### (S)-6-(1-(4-cyano-3-methylphenyl)-5-cyclopentyl-4,5-dihydro-1H-pyrazol-3-yl)-2-methoxynicotinic acid

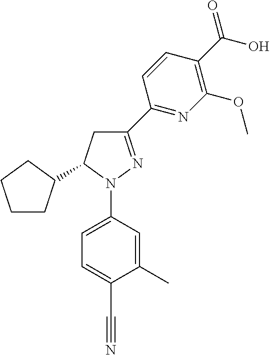

The title compound was prepared from 6-[1-(4-Cyano-3-methyl-phenyl)-5-cyclopentyl-4,5-dihydro-1H-pyrazol-3-yl]-2-methoxy-nicotinic acid (Example 3) using chiral SFC. Column: AD-H, 30×250 mm, 50% methanol/carbon dioxide, 70 mL/min. First eluting peak: chiral HPLC t_{R}=8.610 min (AD-H, 50% methanol/carbon dioxide). 1H NMR (400 MHz, DMSO-d₆) δ ppm 1.04 (1H, br. s.), 1.28 (2H, br. s.), 1.50 (3H, d, J=8.8 Hz), 1.74 (1H, br. s.), 3.18 (2H, d, J=3.7 Hz), 3.22 (6H, d, J=3.7 Hz), 3.96 (3H, s), 4.87 (1H, d, J=11.7 Hz), 7.11 (1H, d, J=7.3 Hz), 7.22 (1H, s), 7.56 (1H, d, J=8.8 Hz), 7.66 (1H, d, J=8.1 Hz), 8.10 (1H, d, J=8.1 Hz)

### Example 6

### 6-[1-(4-cyano-3-methoxy-phenyl)-5-cyclopentyl-4,5-dihydro-1H-pyrazol-3-yl]-2-methoxy-nicotinic acid

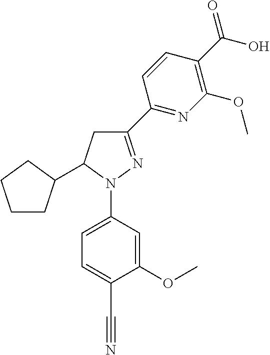

To a solution of methyl 6-(3-cyclopentylacryloyl)-2-methoxynicotinate (Preparation 8, 0.054 g, 0.19 mmol) and 4-hydrazino-2-methoxy-benzonitrile (WO 2008/053300, 0.0522 g, 0.261 mmol) in ethanol (3.8 mL) bubbled with nitrogen was added a solution of 21% sodium ethoxide in ethanol (0.181 g, 0.56 mmol). The mixture was heated to 80° C. for 1 h under nitrogen. The reaction was concentrated and the residue was purified by chromatography (reverse phase, acetonitrile/water) to obtain the title compound (0.0164 g, 21%). 1H NMR (400 MHz, DMSO-d₆) δ ppm 1.02-1.13 (m, 1H) 1.22-1.63 (m, 6H) 1.72-1.82 (m, 1H) 3.49 (dd, J=18.48, 11.53 Hz, 1H) 3.93 (s, 3H) 3.98 (s, 3 H) 4.94 (td, J=7.59, 3.48 Hz, 1H) 6.83 (dd, J=8.60, 1.28 Hz, 1H) 6.94 (d, J=1.10 Hz, 1 H) 7.52 (d, J=8.78 Hz, 1H) 7.71 (d, J=7.69 Hz, 1H) 8.14 (d, J=8.05 Hz, 1H).

### Example 7

### 6-[1-(3-chloro-4-cyano-phenyl)-5-cyclopentyl-4,5-dihydro-1H-pyrazol-3-yl]-2-methoxy-nicotinic acid

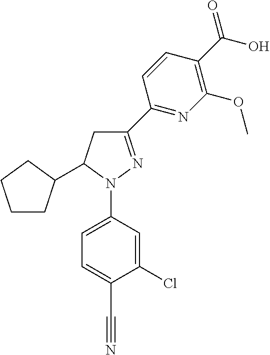

The title compound was prepared by the method used to prepare Example 6 from 6-(3-cyclopentylacryloyl)-2-methoxynicotinate (Preparation 8, 0.258 g, 0.892 mmol) and 2-chloro-4-hydrazino-benzonitrile (WO 2008/053300, 0.255 g, 1.25 mmol). 0.160 g isolated (42.2%). 1H NMR (400 MHz, DMSO-d₆) δ ppm 0.99-1.10 (m, 1H) 1.21-1.64 (m, 6H) 1.73-1.82 (m, 1H) 2.43-2.48 (m, 1H) 3.51 (dd, J=18.48, 11.53 Hz, 1H) 3.98 (s, 3H) 4.94 (td, J=7.59, 3.48 Hz, 1H) 7.24 (dd, J=8.97, 2.01 Hz, 1H) 7.45 (d, J=2.20 Hz, 1H) 7.73 (d, J=6.59 Hz, 1H) 7.75 (d, J=7.69 Hz, 1H) 8.15 (d, J=8.05 Hz, 1H).

### Example 8

### (R)-6-[1-(3-chloro-4-cyano-phenyl)-5-cyclopentyl-4,5-dihydro-1H-pyrazol-3-yl]-2-methoxy-nicotinic acid

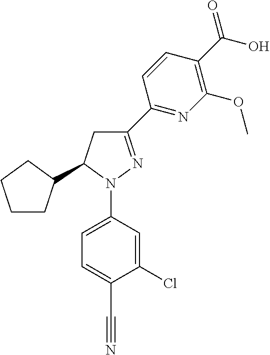

The title compound was prepared from 6-[1-(3-chloro-4-cyano-phenyl)-5-cyclopentyl-4,5-dihydro-1H-pyrazol-3-yl]-2-methoxy-nicotinic acid (Example 7) using chiral SFC. Column AD-H, 30×250 mm, 50% methanol/carbon dioxide, 70 mL/min. First eluting peak t_{R}=7.395 min (AD-H, 50% methanol/carbon dioxide). 1H NMR (400 MHz, DMSO-d₆) δ ppm 1.00-1.10 (m, 1H) 1.22-1.44 (m, 3H) 1.46-1.65 (m, 3H) 1.73-1.83 (m, 1H) 2.44-2.48 (m, 1H) 3.51 (dd, J=18.48, 11.53 Hz, 1H) 3.98 (s, 3H) 4.91-4.97 (m, 1H) 7.25 (d, J=8.78 Hz, 1H) 7.45 (s, 1H) 7.74 (t, J=8.60 Hz, 2H) 8.13 (d, J=7.69 Hz, 1H).

### Example 9

### (S)-6-[1-(3-chloro-4-cyano-phenyl)-5-cyclopentyl-4,5-dihydro-1H-pyrazol-3-yl]-2-methoxy-nicotinic acid

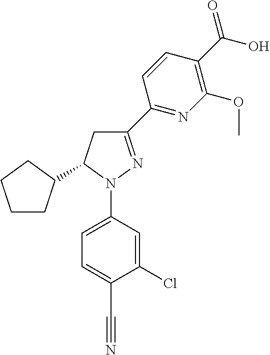

The title compound was prepared from 6-[1-(3-chloro-4-cyano-phenyl)-5-cyclopentyl-4,5-dihydro-1H-pyrazol-3-yl]-2-methoxy-nicotinic acid (Example 7) using chiral SFC. Column AD-H, 30×250 mm, 50% methanol/carbon dioxide, 70 mL/min. Second eluting peak t_{R}=7.384 min (AD-H, 50% methanol/carbon dioxide). 1H NMR (400 MHz, DMSO-d₆) δ ppm 1.00-1.10 (m, 1H) 1.22-1.45 (m, 3H) 1.47-1.65 (m, 3H) 1.72-1.82 (m, 1H) 3.50 (dd, J=18.48, 11.53 Hz, 1H) 3.94 (s, 3H) 4.87-4.94 (m, 1H) 7.22 (d, J=8.42 Hz, 1H) 7.43 (s, 1H) 7.67 (br. s., 1H) 7.73 (d, J=8.78 Hz, 1H) 7.93-8.02 (m, 1H).

### Example 10

### 4-(1-(5-cyano-6-methylpyridin-2-yl)-5-cyclopentyl-4,5-dihydro-1H-pyrazol-3-yl)-2-methoxybenzoic acid

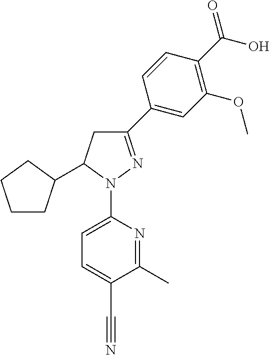

The title compound was prepared by the method used to prepare Example 6 from methyl 4-(3-cyclopentylacryloyl)-2-methoxybenzoate (Preparation 29, 105 mg, 0.364 mmol) and 6-hydrazinyl-2-methylnicotinonitrile (WO 2008/053300, 64.7 mg, 0.436 mmol). 11 mg of the title compound was isolated (7.5%). 1H NMR (400 MHz, DMSO-d₆) δ ppm 1.07-1.18 (m, 1H) 1.34-1.45 (m, 3H) 1.47-1.58 (m, 2H) 1.60-1.72 (m, 2 H) 2.53 (s, 3H) 2.75-2.85 (m, 1H) 3.48 (dd, J=18.12, 11.16 Hz, 1H) 3.91 (s, 3H) 4.98 (ddd, J=11.07, 4.67, 4.39 Hz, 1H) 7.27 (d, J=8.78 Hz, 1H) 7.43 (d, J=8.05 Hz, 1H) 7.47 (s, 1H) 7.70 (d, J=8.05 Hz, 1H) 7.85 (d, J=8.78 Hz, 1H).

### Example 11

### (R)-4-(1-(5-cyano-6-methylpyridin-2-yl)-5-cyclopentyl-4,5-dihydro-1H-pyrazol-3-yl)-2-methoxybenzoic acid

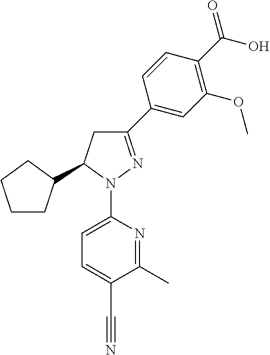

To a solution of (R)-6-(3-chloro-5-cyclopentyl-4,5-dihydro-1H-pyrazol-1-yl)-2-methylnicotinonitrile (Preparation 3, 503 mg, 1.74 mmol) and 4-borono-2-methoxybenzoic acid (355 mg, 1.81 mmol) in 1,2-dimethoxyethane (15 mL) was added 2M sodium carbonate (2.62 mL) followed by tetrakis(triphenylphosphine) palladium (98.2 mg, 0.085 mmol). The reaction mixture was refluxed for 15 h under nitrogen. Preparative HPLC (reverse phase, acetonitrile/water) provided the title compound (248 mg, 35%) as a solid; 1H NMR (400 MHz, DMSO-d₆) δ ppm 1.08-1.18 (m, 1H) 1.32-1.45 (m, 3H) 1.48-1.58 (m, 2H) 1.69 (br. s., 2H) 2.53 (s, 3H) 2.76-2.85 (m, 1H) 3.21 (dd, J=18.48, 4.21 Hz, 1H) 3.49 (dd, J=17.93, 11.71 Hz, 1H) 3.92 (s, 3H) 4.95-5.02 (m, 1H) 7.27 (d, J=8.78 Hz, 1H) 7.43 (d, J=8.05 Hz, 1H) 7.48 (s, 1H) 7.71 (d, J=8.05 Hz, 1H) 7.85 (d, J=8.78 Hz, 1H).

### Example 12

### 6-[1-(5-cyano-6-methyl-pyridin-2-yl)-5-cyclopentyl-4,5-dihydro-1H-pyrazol-3-yl]-2-methoxy-nicotinic acid

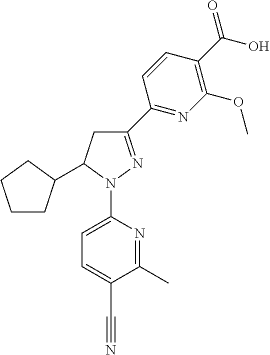

The title compound was prepared by the method used to prepare Example 6 from methyl 6-(3-cyclopentylacryloyl)-2-methoxynicotinate (Preparation 8, 320 mg. 1.11 mmol) and 6-hydrazino-2-methyl-nicotinonitrile (WO 2008/053300, 229 mg, 1.55 mmol). 150 mg of the title compound was isolated (33%) as a solid. 1H NMR (400 MHz, DMSO-d₆) δ ppm 1.08-1.18 (m, 1H) 1.28-1.75 (m, 7H) 2.77-2.87 (m, 1H) 3.15-3.23 (m, 1H) 3.51 (dd, J=18.49, 11.90 Hz, 1H) 3.98 (s, 3H) 4.97-5.03 (m, 1H) 7.26 (d, J=9.15 Hz, 1H) 7.70 (d, J=7.69 Hz, 1H) 7.88 (d, J=9.52 Hz, 1H) 8.14 (d, J=7.69 Hz, 1H.

### Example 13

### (R)-6-(1-(3-chloro-4-cyanophenyl)-5-cyclopentyl-4,5-dihydro-1H-pyrazol-3-yl)-4-methoxynicotinic acid

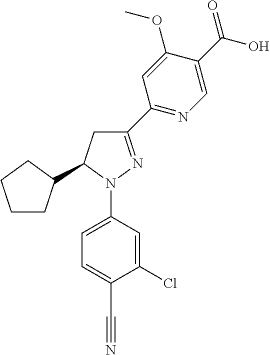

A mixture of methyl 6-chloro-4-methoxynicotinate (Preparation 11, 0.213 g, 1.06 mmol), bis(tributyltin) (0.968 g, 1.58 mmol), and dichlorobis(triphenylphosphine) palladium(II) (0.0722 g, 0.106 mmol) was evacuated and backfilled with argon several times and then anhydrous dioxane (2.1 mL) was added. The mixture was heated to 100° C. for 16 h. Additional dichlorobis(triphenylphosphine) palladium(II) (50 mg) was added and heating was continued at 100° C. for 16 h. The cooled mixture was poured into ethyl acetate, washed with water, washed with brine, dried over magnesium sulfate, and concentrated. Remaining bis(tributyltin) reagent was distilled from the reaction mixture via Kugelrohr. To the residue was added (R)-2-chloro-4-(3-chloro-5-cyclopentyl-4,5-dihydro-1H-pyrazol-1-yl)benzonitrile (Preparation 5, 0.065 g, 0.21 mmol), dichlorobis(triphenylphosphine) palladium(II) (0.0148 g, 0.0211 mmol) and lithium chloride (0.030 g, 0.396 mmol). The mixture was evacuated and backfilled with argon several times, followed by addition of anhydrous toluene (2.1 mL). The mixture was heated to 100° C. for 16 h. After cooling to room temperature, methanol (2 mL) and 2.5N sodium hydroxide (1 mL) were added and the mixture was stirred at room temperature for 2 h. The mixture was cooled to 0° C., poured into a diluted hydrochloride acid solution, and extracted with ethyl acetate. The organic phase was washed with brine, dried over magnesium sulfate, and concentrated. The residue was purified by was purified by chromatography (reverse phase, acetonitrile/water) to obtain the title compound (0.010g, 11%) as a solid. 1H NMR (400 MHz, DMSO-d₆) δ ppm 0.98-1.09 (m, 1H) 1.27-1.38 (m, 3H) 1.47-1.64 (m, 3H) 1.73-1.82 (m, 1H) 3.25 (dd, J=18.67, 3.29 Hz, 1H) 3.49 (dd, J=18.67, 11.35 Hz, 1H) 4.01 (s, 3H) 4.91-4.98 (m, 1H) 7.30 (d, J=9.52 Hz, 1H) 7.50 (s, 1H) 7.72 (s, 1H) 7.75 (d, J=8.79 Hz, 1H) 8.74 (s, 1H).

### Example 14

### (R)-methyl 4-(1-(5-cyano-6-methylpyridin-2-yl)-5-cyclopentyl-4,5-dihydro-1H-pyrazol-3-yl)benzoate

To a solution of (R)-6-(3-chloro-5-cyclopentyl-4,5-dihydro-1H-pyrazol-1-yl)-2-methylnicotinonitrile (Preparation 7, 300 mg, 1.04 mmol) and 4-(methoxycarbonyl)phenylboronic acid (280 mg, 1.56 mmol) in N,N-dimethylformamide (6 mL) was added cesium carbonate (1.05 g, 3.22 mmol) followed by tetrakis(triphenylphosphine) palladium (36.1 mg, 0.031 mmol). The reaction mixture was stirred at 80° C. under argon for 15 h. Chromatography (reverse phase, acetonitrile/water) provided the title compound (153 mg, 38%); 1H NMR (400 MHz, DMSO-d₆) δ ppm 1.10 (1H, br. s.), 1.39 (2H, br. s.), 1.53 (2H, br. s.), 1.69 (1H, br. s.), 2.53 (3H, s), 2.81 (1H, br. s.), 3.21 (1H, dd, J=18.2, 4.5 Hz), 3.33 (2H, s), 3.49 (1H, dd, J=18.2, 11.5 Hz), 3.88 (3H, s), 4.99 (1H, dt, J=11.4, 4.5 Hz), 7.26 (1H, s), 7.87 (1 H, d, J=8.8 Hz), 7.95 (2H, m), 8.03 (2H, m).

### Example 15

### (R)-4-(1-(5-cyano-6-methylpyridin-2-yl)-5-cyclopentyl-4,5-dihydro-1H-pyrazol-3-yl)benzoic acid

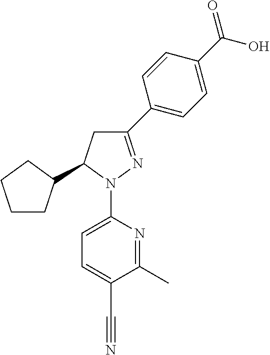

(R)-6-(3-chloro-5-cyclopentyl-4,5-dihydro-1H-pyrazol-1-yl)-2-methylnicotinonitrile (Preparation 7, 200 mg, 0.69 mmol), 4-boronobenzoic acid (115 mg, 0.69 mmol) and palladium tetrakis triphenylphosphine (40 mg, 0.035 mmol) were suspended in acetonitrile (7 mL) and 0.4 M aqueous sodium carbonate (7 mL). The mixture is heated to 90° C. for 4 h. The reaction mixture was cooled and the reaction was concentrated to remove the acetonitrile. To the residue was added ethyl acetate and the layers were separated. The aqueous layer was washed with ethyl acetate three times. The aqueous layer was acidified with concentrated hydrochloric acid and a brownish-green precipitate formed. This precipitate was collected by filtration and dried. The title compound was obtained as a green solid (61 mg, 22%). 1H NMR (400 MHz, METHANOL-d₄) δ ppm 1.16 (1H, d, J=4.1 Hz), 1.32-1.76 (6H, br. m), 1.81 (1H, m), 2.64 (3H, s), 2.81 (1H, br. s.), 3.26 (1H, dd, J=18.1, 4.2 Hz), 3.53 (1H, dd, J=18.0, 11.1

Hz), 5.09 (1H, dt, J=11.2, 4.3 Hz), 7.32 (1H, d, J=8.8 Hz), 7.82 (1H, d, J=9.0 Hz), 7.97 (2H, d, J=8.4 Hz), 8.10 (2H, d, J=8.6 Hz) ppm.

### Example 16

### (R)-4-(1-(5-cyano-6-methylpyridin-2-yl)-5-cyclopentyl-4,5-dihydro-1H-pyrazol-3-yl)benzamide

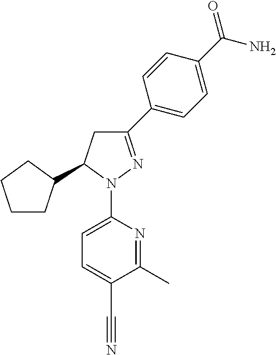

The title compound was prepared by the method used to prepare Example 11 from 4-carbamoylphenylboronic acid (137 mg, 0.832 mmol) and (R)-6-(3-chloro-5-cyclopentyl-4,5-dihydro-1H-pyrazol-1-yl)-2-methylnicotinonitrile (Preparation 7, 200 mg, 0.693 mmol). 63 mg of the title compound was isolated (24%) as a solid. 1H NMR (400 MHz, DMSO-d₆) δ ppm 0.96-1.16 (m, 1H) 1.22-1.75 (m, 7H) 2.50 (s, 3H) 2.78 (br. s., 1H) 3.18 (dd, J=18.07, 4.36 Hz, 1H) 3.37-3.53 (m, 1H) 4.86-5.02 (m, 1H) 7.22 (d, J=8.72 Hz, 1H) 7.45 (s, 1H) 7.77-7.91 (m, 3H) 7.89-7.97 (m, 2H) 8.06 (s, 1H).

### Example 17

### (R)-6-(5-cyclopentyl-3-(4-(methylsulfonyl)phenyl)-4,5-dihydro-1H-pyrazol-1-yl)-2-methylnicotinonitrile

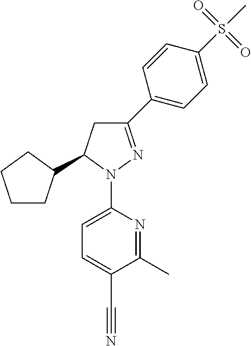

To a solution of (R)-6-(3-chloro-5-cyclopentyl-4,5-dihydro-1H-pyrazol-1-yl)-2-methylnicotinonitrile (Preparation 7, 100 mg, 0.341 mmol) and 4,4,5,5-tetramethyl-2-(4-(methylsulfonyl)phenyl)-1,3,2-dioxaborolane (117 mg, 0.415 mmol) in 1,4-dioxane (10 mL) was added sodium carbonate (129 mg, 1.04 mmol) followed by palladium tetrakis(triphenylphosphine) (0.0613 mg, 0.052 mmol). The reaction mixture was refluxed for 6 h, cooled to room temperature and filtered through celite. Ethyl acetate and water were added and the layers were separated. The organic layer was washed with brine, dried over magnesium sulfate, filtered, and concentrated. The residue was purified by silica gel column chromatography eluting with a gradient of 0-60% ethyl acetate/heptane to obtain the title compound (0.050 g, 35%) as a solid. 1H NMR (500 MHz, CHLOROFORM-d) δ ppm 0.93 (1H, m), 1.17 (1H, m), 1.31 (1H, m), 1.57-1.61 (3H, m), 1.85 (1H, br. s.), 2.84 (3H, m), 2.94 (1H, br. s.), 3.12 (4H, m), 3.42 (1H, m), 4.18 (1H, m), 5.12 (1H, m), 7.22 (1H, d, J=8.5 Hz), 7.62 (1H, d, J=8.2 Hz 7.98 (2H, m), 8.03 (2H, m).

### Example 18

### (R)-6-(5-cyclopentyl-3-(3-methyl-4-oxo-3,4-dihydroquinazolin-6-yl)-4,5-dihydro-1H-pyrazol-1-yl)-2-methylnicotinonitrile

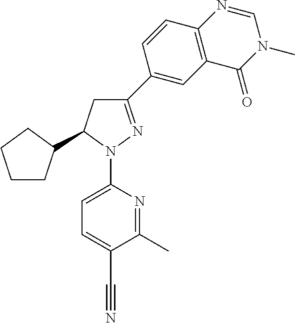

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 200 mg, 0.693 mmol) and 3-methyl-6-(4,4,5,5-tetramethyl-1,3,2-dioxaborolan-2-yl)quinazolin-4(3H)-one (117 mg, 0.415 mmol). 38.6 mg of the title compound was isolated (36%) as a solid. ¹H NMR (500 MHz, DMSO-d₆) δ ppm: 8.42 (s, 1H), 8.39 (s, 1H), 8.30 (d, 1H), 7.85 (d, 1H), 7.72 (d, 1H), 7.26 (d 1H), 5.00 (m, 1H), 3.52 (m, 4H), 3.32 (m, 1H), 2.82 (m, 1H), 2.54 (s, 3H), 1.71-1.11 (m, 8H).

### Example 19

### (R)-6-(5-cyclopentyl-3-(3-oxo-3,4-dihydro-2H-benzo[b][1,4]oxazin-6-yl)-4,5-dihydro-1H-pyrazol-1-yl)-2-methylnicotinonitrile

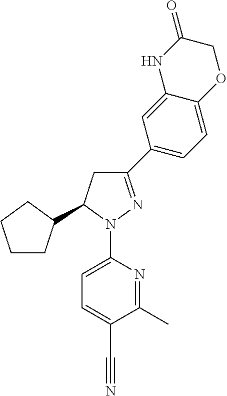

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 50 mg, 0.17 mmol) and 6-(4,4,5,5-tetramethyl-1,3,2-dioxaborolan-2-yl)-2H-benzo[b][1,4]oxazin-3(4H)-one (Preparation 13, 57.2 mg, 0.208 mmol). 23 mg isolated (33%) as a solid. 1H NMR (400 MHz, METHANOL-d₄) δ ppm 1.15 (1H, m), 1.46 (1H, m), 1.56 (1H, m), 1.56 (1H, d, J=8.7 Hz), 1.73 (1H, dd, J=7.5, 4.2 Hz), 2.52 (2H, s), 2.86 (1H, d, J=4.6 Hz), 3.04 (1H, dd, J=17.9, 4.6 Hz), 3.29 (4H, m), 4.60 (2H, s), 4.97 (1H, ddd, J=11.4, 4.4, 4.2 Hz), 6.97 (1H, d, J=8.3 Hz), 7.17 (1H, d, J=8.7 Hz), 7.37 (1H, s), 7.34 (1H, d, J=2.1 Hz), 7.63 (1H, d, J=9.1 Hz).

### Example 20

### (R)-4-(1-(5-cyano-6-methylpyridin-2-yl)-5-cyclopentyl-4,5-dihydro-1H-pyrazol-3-yl)benzenesulfonamide

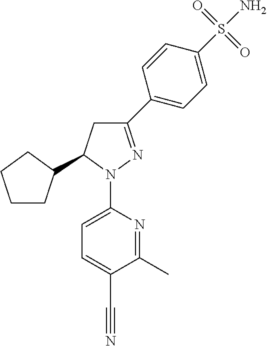

To a solution of ((R)-6-(3-chloro-5-cyclopentyl-4,5-dihydro-1H-pyrazol-1-yl)-2-methylnicotinonitrile (Preparation 7, 150 mg, 0.530 mmol) and 4-(4,4,5,5-tetramethyl-1,3,2-dioxaborolan-2-yl)benzenesulfonamide (153 mg, 0.530 mmol) in 1,4-dioxane (10 mL) was added cesium carbonate (517 mg, 1.59 mmol) followed by tetrakis(triphenylphosphine)palladium (0.092 mg, 0.079 mmol). The reaction mixture was heated to reflux for 16 h, cooled to room temperature and filtered through celite. Ethyl acetate and water were added and the layers were separated. The organic layer was washed with brine, dried over magnesium sulfate, filtered, and concentrated. The residue was purified by silica gel column chromatography eluting with a gradient of 0%-50% ethyl acetate/heptane to obtain the title compound (0.095 g, 44%) as a solid. ¹H NMR (500 MHz, CHLOROFORM-d) δ ppm 0.93 (1H, m), 1.17 (1H, m), 1.31 (1H, m), 1.57-1.61 (3H, m), 1.85 (1H, br. s.), 2.84 (3H, m), 2.94 (1H, br. s.), 3.12 (1H, m), 3.42 (1H, m), 4.18 (1H, m), 4.81 (2H, s), 5.12 (1H, m), 7.22 (1H, d, J=8.5 Hz), 7.62 (1H, d, J=8.2 Hz 7.98 (2H, m), 8.03 (2H, m).

### Example 21

### (R)-6-(5-cyclopentyl-3-(3-(methylsulfonyl)phenyl)-4,5-dihydro-1H-pyrazol-1-yl)-2-methylnicotinonitrile

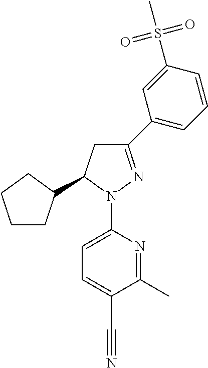

The title compound was prepared by the method used to prepare Example 17 from ((R)-6-(3-chloro-5-cyclopentyl-4,5-dihydro-1H-pyrazol-1-yl)-2-methylnicotinonitrile (Preparation 7, 200 mg, 0.693 mmol) and 3-(methylsulfonyl)phenylboronic acid (155 mg, 0.693 mmol). 0.220 g of the title compound was isolated (77.7%) as a solid. 1H NMR (500 MHz, CHLOROFORM-d) δ ppm 0.93 (1H, m), 1.17 (1H, m), 1.31 (1H, m), 1.57-1.61 (3H, m), 1.85 (1H, br. s.), 2.84 (3H, m), 2.94 (1H, br. s.), 3.12 (4H, m), 3.42 (1H, m), 4.18 (1H, m), 5.12 (1H, m), 7.28 (1H, m), 7.62 (2H, m), 7.98 (1H, m), 8.03 (1H, m), 8.25 (1H, s).

### Example 22

### (R)-6-(5-cyclopentyl-3-(4-(ethylsulfonyl)phenyl)-4,5-dihydro-1H-pyrazol-1-yl)-2-methylnicotinonitrile

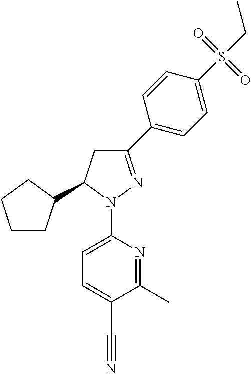

The title compound was prepared by the method used to prepare Example 20 from (R)-6-(3-chloro-5-cyclopentyl-4,5-dihydro-1H-pyrazol-1-yl)-2-methylnicotinonitrile (Preparation 7, 150 mg, 0.519 mmol) and 4-(ethylsulfonyl)phenylboronic acid (111 mg, 0.519 mmol). 0.105 g of the title compound was isolated (48%) as a solid. 1H NMR (500 MHz, CHLOROFORM-d) δ ppm 0.93 (1H, m), 1.17 (1H, m), 1.31 (4H, m), 1.57-1.61 (3H, m), 1.85 (1H, br. s.), 2.84 (3H, m), 2.94 (1H, br. s.), 3.12 (1H, m), 3.18 (2 H, m), 3.42 (1H, m), 4.18 (1H, m), 5.12 (1H, m), 7.22 (1H, d, J=8.5 Hz), 7.62 (1H, d, J=8.2 Hz), 7.98 (4H, m).

### Example 23

### (R)-6-(3-(4-cyano-3-methylphenyl)-5-cyclopentyl-4,5-dihydro-1H-pyrazol-1-yl)-2-methylnicotinonitrile

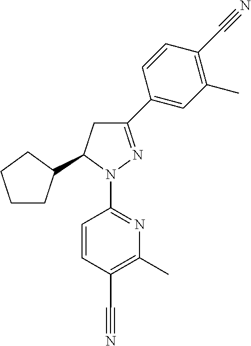

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 200 mg, 0.693 mmol) and 2-methyl-4-(4,4,5,5-tetramethyl-1,3,2-dioxaborolan-2-yl)benzonitrile (Preparation 14, 177 mg, 0.728 mmol). 213 mg isolated (83%) as a light green-yellow solid. ¹H NMR (500 MHz, DMSO-d₆) δ ppm: 7.83 (m, 4H), 7.28 (d, 1H), 4.99 (m, 1H), 3.46 (dd, 1H), 3.23 (dd, 1H), 2.80 (m, 1H), 1.72-1.04 (m, 8H).

### Example 24

### (R)-4-(1-(5-cyano-6-methylpyridin-2-yl)-5-cyclopentyl-4,5-dihydro-1H-pyrazol-3-yl)-2-methylbenzamide

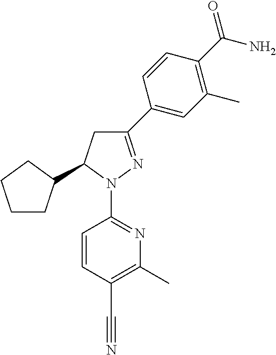

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 200 mg, 0.693 mmol) and 2-methyl-4-(4,4,5,5-tetramethyl-1,3,2-dioxaborolan-2-yl)benzamide (Preparation 15, 190 mg, 0.728 mmol). 158 mg of the title compound was isolated (59%) as a off white solid. ¹H NMR (500 MHz, DMSO-d₆) δ ppm: 7.84 (d, 1H), 7.76 (s, 1H), 7.66 (m, 2H), 7.43 (m, 2H), 7.23 (d, 1H), 4.96 (m, 1H), 3.44 (dd, 1H), 3.18 (dd, 1H), 2.80 (m, 1H), 2.52 (s, 3H), 2.42 (s, 3H), 1.71-1.07 (m, 8H).

### Example 25

### (R)—N-(4-(1-(5-cyano-6-methylpyridin-2-yl)-5-cyclopentyl-4,5-dihydro-1H-pyrazol-3-yl)phenyl)methanesulfonamide

The title compound was prepared by the method used to prepare Example 17 from (R)-6-(3-chloro-5-cyclopentyl-4,5-dihydro-1H-pyrazol-1-yl)-2-methylnicotinonitrile (Preparation 7, 200 mg, 0.693 mmol) and 4-(methylsulfonamido) phenylboronic acid (149 mg, 0.693 mmol). 0.080 g of the title compound was isolated (27%) as a solid. 1H NMR (500 MHz, CHLOROFORM-d) δ ppm 0.89 (1H, m), 1.17 (1H, m), 1.31 (1H, m), 1.57-1.61 (3H, m), 1.85 (1H, br. s.), 2.84 (3H, m), 2.94 (1H, br. s.), 3.12 (4H, m), 3.42 (1H, m), 4.18 (1H, m), 5.02 (1H, m), 5.12 (1H, m), 7.22 (1H, d, J=8.5 Hz), 7.62 (1H, d, J=8.2 Hz 7.98 (2H, m), 8.00 (2H, m).

### Example 26

### (R)-6-(3-(4-cyano-3-methoxyphenyl)-5-cyclopentyl-4,5-dihydro-1H-pyrazol-1-yl)-2-methylnicotinonitrile

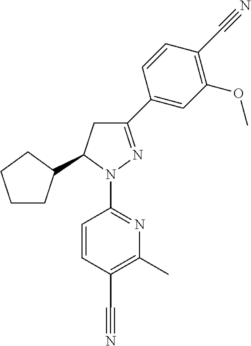

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 150 mg, 0.519 mmol) and 2-methoxy-4-(4,4,5,5-tetramethyl-1,3,2-dioxaborolan-2-yl)benzonitrile (Preparation 16, 141 mg, 0.545 mmol). 24 mg of the title compound was isolated as a yellow solid (12%). ¹H NMR (500 MHz, DMSO-d₆) δ ppm: 7.85 (d, 1H), 7.77 (d, 1H), 7.47 (m, 2H), 7.27 (d, 1H), 4.97 (m, 1H), 3.98 (s, 3H), 3.45 (dd, 1H), 3.23 (dd, 1H), 2.78 (m, 1H), 2.50 (s, 3H), 2.68-1.04 (m, 8H).

### Example 27

### (R)-4-(1-(6-cyano-6-methylpyridin-2-yl)-5-cyclopentyl-4,5-dihydro-1H-pyrazol-3-yl)-2-methoxybenzamide

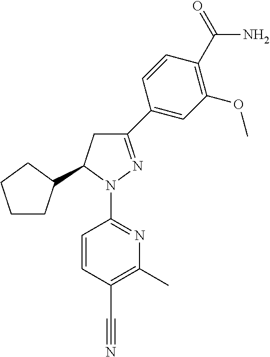

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 150 mg, 0.519 mmol) and 2-methoxy-4-(4,4,5,5-tetramethyl-1,3,2-dioxaborolan-2-yl)benzamide (Preparation 17, 151 mg, 0.545 mmol). 104 mg of the title compound was isolated (50%) as a yellow solid. ¹H NMR (500 MHz, DMSO-d₆) δ ppm: 7.86 (m, 2H), 7.67 (s, 1H), 7.60 (s, 1H), 7.45 (m, 2H), 7.27 (d, 1H), 4.96 (m, 1H), 3.98 (s, 3H), 3.44 (dd, 1H), 3.23 (dd, 1H), 2.80 (m, 1H), 2.52 (s, 3H), 1.71-1.07 (m, 8H).

### Example 28

### (R)-6-(5-cyclopentyl-3-(6-methoxypyridin-2-yl)-4,5-dihydro-1H-pyrazol-1-yl)-2-methylnicotinonitrile

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 0.200 g, 0.693 mmol) and 2-methoxy-6-(4,4,5,5-tetramethyl-[1,3,2]dioxaborolan-2-yl)-pyridine (0.179 g, 0.762 mmol). 0.126 g of the title compound was isolated (50%) as a white solid. ¹H NMR (500 MHz, DMSO) δ ppm 7.86 (d, 1H), 7.80 (m, 1H), 7.65 (d, 1H), 7.22 (d, 1H), 6.86 (d, 1H), 4.96 (m, 1H), 3.91 (s, 3H), 3.48 (dd, 1H), 3.20 (dd, 1H), 2.80 (m, 1H), 2.53 (s, 3H), 1.71-1.10 (m, 8H).

### Example 29

### (R)-3-(1-(5-cyano-6-methylpyridin-2-yl)-5-cyclopentyl-4,5-dihydro-1H-pyrazol-3-yl)benzamide

A mixture of (R)-6-(3-chloro-5-cyclopentyl-4,5-dihydro-1H-pyrazol-1-yl)-2-methylnicotinonitrile (Preparation 7, 100 mg, 0.35 mmol), 3-carbamoylphenylboronic acid (57 mg, 0.35 mmol), sodium carbonate (110 mg, 1.0 mmol), tetrakis(triphenylphosphine)palladium (20 mg, 0.02 mmol), 1,2-dimethoxyethane (2 mL) and water (1 mL) was stirred at 100° C. for 16 h. The reaction was cooled to room temperature diluted with ethyl acetate and water. The aqueous layer was extracted 3 times with ethyl acetate and the combined organic layers were dried over magnesium sulfate, filtered and concentrated. The residue was purified by silica gel column chromatography eluting with a gradient of 0-100% ethyl acetate/heptane to afford the title compound as a white solid (45.8 mg, 35.4%). 1H NMR (400 MHz, CHLOROFORM-d) δ ppm 1.14 (1H, dd, J=13.9, 4.3 Hz), 1.22 (1H, t, J=7.0 Hz), 1.33 (1H, dd, J=12.7, 9.6 Hz), 1.52 (1H, m), 1.51 (1H, d, J=2.9 Hz), 1.66 (1H, d, J=4.3 Hz), 1.78 (1H, t, J=7.8 Hz), 2.62 (3H, s), 2.87 (1H, d, J=4.9 Hz), 3.09 (1H, dd, J=17.5, 4.6 Hz), 3.38 (1 H, dd, J=17.6, 11.5 Hz), 5.06 (1H, dt, J=11.5, 4.7 Hz), 7.25 (1H, s), 7.52 (1H, t, J=7.8 Hz), 7.60 (1H, d, J=8.8 Hz), 7.80 (1H, ddd, J=8.0, 1.4, 1.2 Hz), 7.97 (1H, dt, J=7.8, 1.4 Hz), 8.20 (1H, t, J=1.6 Hz).

### Example 30

### (R)-6-(5-cyclopentyl-3-(1-oxoisoindolin-5-yl)-4,5-dihydro-1H-pyrazol-1-yl)-2-methylnicotinonitrile

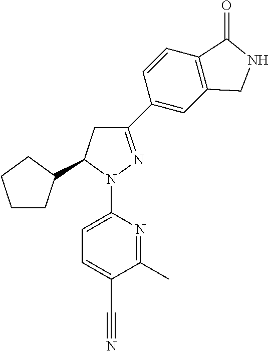

The title compound was prepared by the method used to prepare Example 29 from (R)-6-(3-chloro-5-cyclopentyl-4,5-dihydro-1H-pyrazol-1-yl)-2-methylnicotinonitrile (Preparation 7, 70 mg, 0.24 mmol) and 5-(4,4,5,5-tetramethyl-1,3,2-dioxaborolan-2-yl)isoindolin-1-one (63 mg, 0.24 mmol). 5.6 mg of the title compound was isolated as a white solid (6%). 1H NMR (400 MHz, DMSO-d₆) δ ppm 1.04 (1H, s), 1.54 (1H, m), 1.25-2.79 (6H, br. m.), 2.79 (1H, br. s.), 3.21 (1H, m), 3.29 (3H, s), 3.47 (1H, m), 4.40 (2H, s), 4.98 (1H, m), 7.23 (1H, d), 7.71 (1H, d), 7.83 (1H, d), 7.90 (1H, d), 7.99 (1H, s), 8.64 (1H, s) ppm.

### Example 31

### (R)-6-(5-cyclopentyl-3-(3-oxoisoindolin-5-yl)-4,5-dihydro-1H-pyrazol-1-yl)-2-methylnicotinonitrile

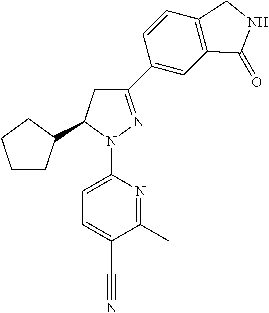

The title compound was prepared by the method used to prepare Example 29 from (R)-6-(3-chloro-5-cyclopentyl-4,5-dihydro-1H-pyrazol-1-yl)-2-methylnicotinonitrile (Preparation 7, 100 mg, 0.35 mmol) and 6-(4,4,5,5-tetramethyl-1,3,2-dioxaborolan-2-yl)isoindolin-1-one (90 mg, 0.35 mmol). 48 mg of the title compound was isolated as a yellow solid (36%). 1H NMR (400 MHz, DMSO-d₆) δ ppm 1.10 (2H, m), 1.27-1.71 (6H, br. m.), 2.81 (1H, br. s.), 3.24 (1H, m), 3.30 (3H, s), 3.48 (1H, m), 4.43 (2H, s), 4.97 (1H, dt, J=11.3, 4.5 Hz), 7.24 (1H, d, J=8.8 Hz), 7.66 (1H, d, J=8.0 Hz), 7.83 (1H, d, J=8.8 Hz), 8.02 (1H, s), 8.10 (1H, dd, J=8.0, 1.6 Hz), 8.66 (1H, s).

### Example 32

### (R)-methyl 6-(1-(5-cyano-6-methylpyridin-2-yl)-5-cyclopentyl-4,5-dihydro-1H-pyrazol-3-yl)-2-methoxynicotinate

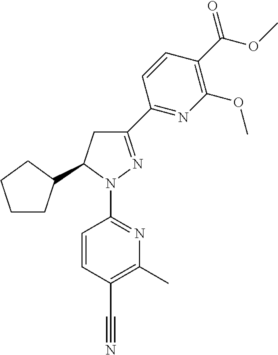

The title compound was prepared by the method used to prepare Example 29 from (R)-6-(3-chloro-5-cyclopentyl-4,5-dihydro-1H-pyrazol-1-yl)-2-methylnicotinonitrile (Preparation 7, 90 mg, 0.31 mmol) and methyl 2-methoxy-6-(4,4,5,5-tetramethyl-[1,3,2]dioxaborolan-2-yl)-nicotinate (Preparation 10, 100 mg, 0.34 mmol). 50 mg of the title compound was isolated as a yellow solid (38% yield). 1H NMR (500 MHz, DMSO-d₆) δ ppm 1.09 (2H, t, J=7.0 Hz), 1.42 (2H, m), 1.52 (1H, d, J=3.4 Hz), 1.61 (1H, br. s.), 1.70 (1H, d, J=7.6 Hz), 2.54 (3H, s), 2.83 (1H, br. s.), 3.20 (1H, dd, J=18.7, 4.8 Hz), 3.32 (3H, s), 3.38 (1H, q, J=7.1 Hz), 3.51 (1H, dd, J=18.5, 11.7 Hz), 3.88 (3H, s), 5.00 (1H, dt, J=11.6, 4.7 Hz), 7.27 (1H, d, J=8.8 Hz), 7.72 (1H, s), 7.90 (1H, d, J=8.8 Hz), 8.17 (1H, d, J=7.8 Hz).

### Example 33

### (R)-6-(1-(5-cyano-6-methylpyridin-2-yl)-5-cyclopentyl-4,5-dihydro-1H-pyrazol-3-yl-methoxynicotinic acid

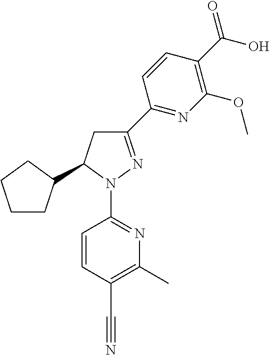

To a solution of (R)-methyl 6-(1-(5-cyano-6-methylpyridin-2-yl)-5-cyclopentyl-4,5-dihydro-1H-pyrazol-3-yl)-2-methoxynicotinate (Example 32, 0.30 g, 0.715 mmol) in tetrahydrofuran (3 mL) was added 2M aqueous lithium hydroxide (0.536 mL, 0.0262 g, 1.07 mmol). The reaction was heated to 40° C. for 16 h. The mixture was cooled to room temperature, diluted with water, acidified to pH=4 with 1N aqueous hydrochloric acid and extracted with dichloromethane 3 times. The combined organic layers were washed with brine, dried over magnesium sulfate, filtered and concentrated. From the concentrated mixture the title compound was filtered and isolated as a yellow solid (0.202 g, 70%). 1H NMR (500 MHz, DMSO-d₆) δ ppm 1.09 (2H, t, J=7.0 Hz), 1.42 (2H, m), 1.52 (1H, d, J=3.4 Hz), 1.61 (1H, br. s.), 1.70 (1H, d, J=7.6 Hz), 2.54 (3H, s), 2.83 (1H, br. s.), 3.20 (1H, dd, J=18.7, 4.8 Hz), 3.32 (3H, s), 3.38 (1H, q, J=7.1 Hz), 3.51 (1H, dd, J=18.5, 11.7 Hz), 5.00 (1H, dt, J=11.6, 4.7 Hz), 7.27 (1H, d, J=8.8 Hz), 7.72 (1H, s), 7.90 (1H, d, J=8.8 Hz), 8.17 (1H, d, J=7.8 Hz), 13.00 (1H, s).

### Example 34

### Methyl 6-(1-(3-chloro-4-cyanophenyl)-5-cyclopentyl-4,5-dihydro-1H-pyrazol-3-yl)nicotinate

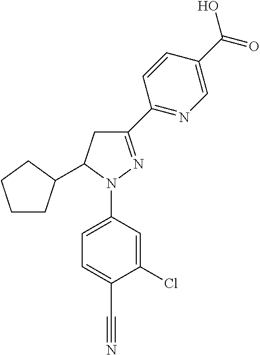

The title compound was prepared by the method used to prepare Example 6 from methyl 6-(3-cyclopentylacryloyl)nicotinate (Preparation 9, 60 mg, 0.23 mmol) and 2-chloro-4-hydrazinylbenzonitrile (WO 2008/053300, 66 mg, 0.32 mmol). 24 mg of the title compound was isolated (26%) as a solid. 1H NMR (400 MHz, DMSO-d₆) δ ppm 0.97-1.08 (m, 1H) 1.22-1.45 (m, 3H) 1.45-1.65 (m, 3H) 1.73-1.82 (m, 1H) 3.52 (dd, J=18.30, 11.35 Hz, 1H) 4.96 (td, J=7.60, 3.48 Hz, 1H) 7.27 (dd, J=8.79, 1.83 Hz, 1H) 7.49 (d, J=1.46 Hz, 1H) 7.76 (d, J=8.79 Hz, 1H) 8.21 (d, J=8.42 Hz, 1H) 8.29 (dd, J=8.42, 2.20 Hz, 1H) 9.09 (s, 1H).

### Example 35

### (R)-methyl 5-(1-(5-cyano-6-methylpyridin-2-yl)-5-cyclopentyl-4,5-dihydro-1H-pyrazol-3-yl)-2-(methylsulfonyl)benzoate

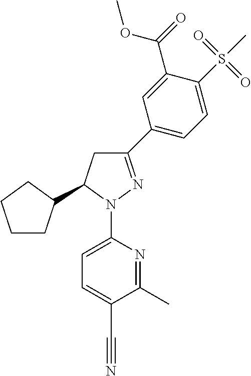

The title compound was prepared by the method used to prepare Example 20 from (R)-6-(3-chloro-5-cyclopentyl-4,5-dihydro-1H-pyrazol-1-yl)-2-methylnicotinonitrile (Preparation 7, 180 mg, 0.623 mmol) and methyl 2-(methylsulfonyl)-5-(4,4,5,5-tetramethyl-1,3,2-dioxaborolan-2-yl)benzoate (Preparation 20, 212 mg, 0.623 mmol). 0.20 g of the title compound was isolated (41.3%) as a solid. 1H NMR (500 MHz, CHLOROFORM-d) δ ppm 0.93 (1H, m), 1.17 (1H, m), 1.31 (1H, m), 1.57-1.71 (2H, m), 1.85 (1H, br. m.), 2.62 (3H, s), 2.90 (1H, m), 3.04 (1H, dd), 3.40 (4H, m), 3.42 (1 H, m), 4.02 (3H, s), 4.18 (1H, m), 5.12 (1H, m), 7.28 (1H, m), 7.62 (1H, d), 7.98 (1H, d), 8.03 (1H, s), 8.20 (1H, d).

### Example 36

### (R)-5-(1-(5-cyano-6-methylpyridin-2-yl)-5-cyclopentyl-4,5-dihydro-1H-pyrazol-3-yl)-2-methoxybenzoic acid

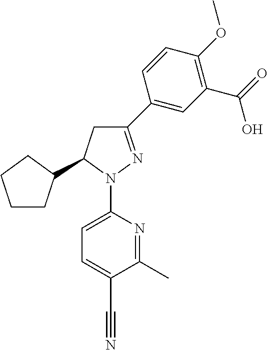

1,1′-Bis(diphenylphospino)ferrocene-palladium dichloride (27.6 mg, 0.037 mmol), methyl 5-bromo-2-methoxybenzoate (180 mg, 0.734 mmol), bis(pinacolato)diboron (224 mg, 0.881 mmol) and potassium acetate (223 mg, 2.20 mmol) were combined in a microwave vial with degassed 1,4-dioxane (3 mL). The vial was sealed and heated at 100° C. for 20 min in a microwave reactor. The reaction mixture was cooled to room temperature, diluted with ethyl acetate (100 mL) and filtered through celite pad to give an orange solution. The filtrate was washed with water (50 mL), dried over magnesium sulfate, filtered, and concentrated to give crude methyl 2-methoxy-5-(4,4,5,5-tetramethyl-1,3,2-dioxaborolan-2-yl)benzoate as an orange oil. This intermediate was combined with (R)-6-(3-chloro-5-cyclopentyl-4,5-dihydro-1H-pyrazol-1-yl)-2-methylnicotinonitrile (Preparation 7, 180 mg, 0.623 mmol), tetrakis(triphenylphosphine)palladium (36.6 mg, 0.031 mmol) and 1,2-dimethoxyethane (5 mL). 2M aqueous sodium carbonate (0.685 mL, 1.37 mmol) was added and the dark brown mixture was heated at 88° C. for 5 h. The solution was cooled to room temperature, diluted with ethyl acetate (150 mL), filtered through celite, and washed with water (50 mL). The organic layer was dried over magnesium sulfate, filtered, and concentrated to give of crude (R)-methyl 5-(1-(5-cyano-6-methylpyridin-2-yl)-5-cyclopentyl-4,5-dihydro-1H-pyrazol-3-yl)-2-methoxybenzoate as an orange oil (250 mg, 95.9%). This intermediate was dissolved in tetrahydrofuran (10 mL) and water (2 mL). Lithium hydroxide monohydrate (103 mg, 2.39 mmol) was added and the mixture was stirred at room temperature for 3 h then heated to 60° C. for 16 hours. The mixture was cooled to room temperature. The reaction was concentrated and the residue was dissolved in water (10 mL). The solution was acidified to pH=3 with 1N aqueous hydrochloric acid and the resulting off-white precipitate was collected and dried. The solid was purified by silica gel column chromatography eluting with a gradient of 0%-100% ethyl acetate/heptane to obtain a clear oil that was stirred in diethyl ether (10 mL) at room temperature producing a fine yellow powder which was collected and dried to give the title compound (40 mg, 17%). 1H NMR (400 MHz, CHLOROFORM-d) δ ppm 0.99-1.40 (m, 2H) 1.53 (br. s., 5H) 1.77 (br. s., 1H) 2.59 (s, 3H) 2.85 (br. s., 1H) 3.05 (dd, J=17.66, 4.58 Hz, 1H) 3.33 (dd, J=17.56, 11.51 Hz, 1H) 4.14 (s, 3H) 4.95-5.10 (m, 1H) 7.13 (d, J=8.78 Hz, 1H) 7.16-7.26 (m, 1H) 7.57 (d, J=8.97 Hz, 1H) 8.14 (dd, J=8.78, 2.34 Hz, 1H) 8.36 (d, J=2.34 Hz, 1H) 10.60 (s, 1H).

### Example 37

### (R)-methyl 4-(1-(5-cyano-6-methylpyridin-2-yl)-5-cyclopentyl-4,5-dihydro-1H-pyrazol-3-yl)-2-ethoxybenzoate

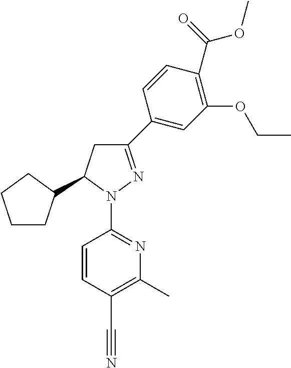

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 200 mg, 0.693 mmol) and methyl 2-ethoxy-4-(4,4,5,5-tetramethyl-1,3,2-dioxaborolan-2-yl)benzoate (Preparation 18, 333 mg, 0.762 mmol). 207 mg of the title compound was isolated (69%) as a white solid. ¹H NMR (400 MHz, DMSO-d₆) δ ppm 7.86 (d, 1H), 7.71 (d, 1H), 7.45 (m, 2H), 7.26 (d, 1H), 4.97 (m, 1H), 4.21 (m, 2H), 3.80 (s, 3H), 3.46 (dd, 1H), 3.22 (dd, 1H), 2.80 (m, 1H), 2.52 (s, 3H), 1.72-1.06 (m, 11H).

### Example 38

### (R)-methyl 4-(1-(5-cyano-6-methylpyridin-2-yl)-5-cyclopentyl-4,5-dihydro-1H-pyrazol-3-yl)-3-methoxybenzoate

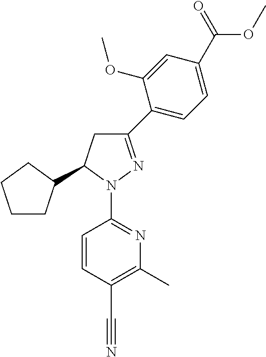

The title compound was prepared by the method used to prepare Example 11 from (R)-6-(3-chloro-5-cyclopentyl-4,5-dihydro-1H-pyrazol-1-yl)-2-methylnicotinonitrile(Preparation 7, 200 mg, 0.693 mmol) and methyl 3-methoxy-4-(4,4,5,5-tetramethyl-1,3,2-dioxaborolan-2-yl)benzoate (Preparation 19, 202 mg, 0.693 mmol). 140 mg of the title compound was isolated as a yellow solid (48%). 1H NMR (400 MHz, DMSO-d₆) δ ppm 1.08-1.18 (m, 1H) 1.23-1.70 (m, 7H) 2.67-2.78 (m, 1 H) 3.18 (dd, 1H) 3.29 (s, 3H) 3.48-3.58 (m, 1H) 3.86 (s, 3H) 3.90 (s, 3H) 4.87-4.94 (m, 1H) 7.14-7.18 (m, 1H) 7.56-7.59 (m, 2H) 7.81 (dd, 1H) 7.92 (dd, 1H).

### Example 39

### (R)-methyl 4-(1-(5-cyano-6-methylpyridin-2-yl)-5-cyclopentyl-4,5-dihydro-1H-pyrazol-3-yl)-3-methoxybenzoic acid

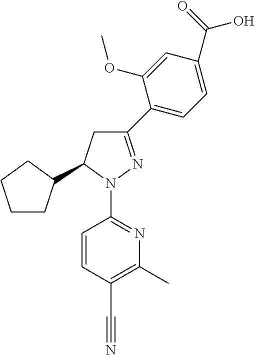

The title compound was prepared by the method used to prepare Example 33 from (R)-methyl 4-(1-(5-cyano-6-methylpyridin-2-yl)-5-cyclopentyl-4,5-dihydro-1H-pyrazol-3-yl)-3-methoxybenzoate (Example 38, 131 mg, 0.281 mmol). 54 mg of the title compound was isolated as a yellow solid (47%). 1H NMR (400 MHz, DMSO-d₆) δ ppm 1.22-1.72 (m, 8H) 2.63-2.79 (m, 1H) 3.11-3.24 (m, 1H) 3.28 (s, 3H) 3.46-3.59 (m, 1H) 3.89 (s, 3H) 4.84-4.96 (m, 1H) 7.11-7.20 (m, 1H) 7.51-7.60 (m, 2H) 7.75-7.93 (m, 2 H) 13.16 (br. s., 1H).

### Example 40

### (R)-6-(1-(5-cyano-6-methylpyridin-2-yl)-5-cyclopentyl-4,5-dihydro-1H-pyrazol-3-yl)-2-methoxynicotinamide

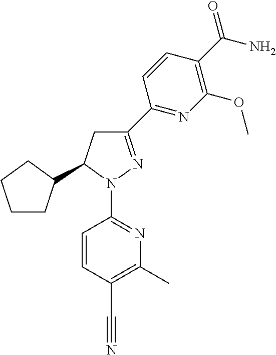

To a solution of (R)-6-(3-chloro-5-cyclopentyl-4,5-dihydro-1H-pyrazol-1-yl)-2-methylnicotinonitrile (Preparation 7, 0.175 g, 0.606 mmol) and 2-methoxy-6-(4,4,5,5-tetramethyl-[1,3,2]dioxaborolan-2-yl)-nicotinamide (Preparation 21, 0.202 g, 0.727 mmol) in dichloromethane (3 mL) was added 2M sodium carbonate (0.67 mL, 0.142 g, 1.33 mmol), followed by tetrakis(triphenylphosphine)palladium (0.0347 g, 0.03 mmol). The reaction mixture was refluxed for 16 h. The reaction was cooled to room temperature and filtered through celite. To the filtrate was added ethyl acetate and water. The layers were separated and the organic layer was washed with brine (10 mL) and dried over magnesium sulfate. Silica gel was added to the filtrate and the mixture was concentrated. The residue was purified by silica gel column chromatography eluting with a gradient of 0%-8.6% methanol/dichloromethane with ammonium hydroxide to obtain the title compound (0.0345 g, 14%) as a yellow solid. ¹H NMR (DMSO-d₆) δ ppm 8.23 (d, 1H), 7.89 (d, 1H), 7.75 (m, 3H), 7.26 (d, 1H), 5.00 (m, 1H), 4.04 (s, 3H), 3.52 (dd, 1H), 3.20 (dd, 1H), 2.80 (m, 1H), 2.54 (s, 3H), 1.72-1.07 (m, 8H).

### Example 41

### (R)-4-(1-(5-cyano-6-methylpyridin-2-yl)-5-cyclopentyl-4,5-dihydro-1H-pyrazol-3-yl)-2-ethoxybenzoic acid

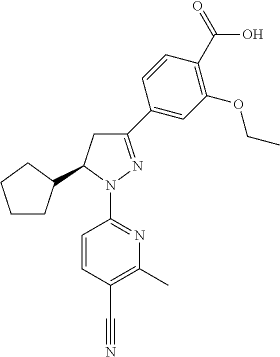

The title compound was prepared by the method used to prepare Example 33 from (R)-methyl 4-(1-(5-cyano-6-methylpyridin-2-yl)-5-cyclopentyl-4,5-dihydro-1H-pyrazol-3-yl)-2-ethoxybenzoate (Example 37, 140 mg, 0.324 mmol). 99.6 mg of the title compound was isolated (74%) as a yellow solid. ¹H NMR (DMSO-d₆) δ ppm 12.7 (bs, 1H), 7.85 (d, 1H), 7.68 (d, 1H), 7.43 (m, 2H), 7.26 (d, 1H), 4.96 (m, 1H), 4.16 (m, 2H), 3.45 (dd, 1H), 3.23 (dd, 1H), 2.81 (m, 1H), 2.52 (s, 3H), 1.71-1.07 (m, 11H).

### Example 42

### (R)-4-(1-(5-cyano-6-methylpyridin-2-yl)-5-cyclopentyl-4,5-dihydro-1H-pyrazol-3-yl)-2-ethoxybenzamide

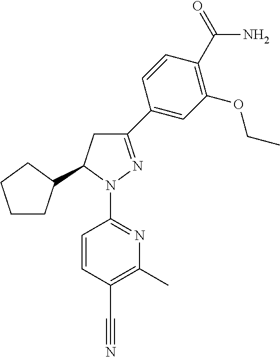

To a solution of (R)-4-(1-(5-cyano-6-methylpyridin-2-yl)-5-cyclopentyl-4,5-dihydro-1H-pyrazol-3-yl)-2-ethoxybenzoic acid (Example 41, 60 mg, 0.14 mmol) in N,N-dimethylformamide (1.5 mL) was added 1,1′-carbonyldiimidazole (29.0 mg, 0.179 mmol). The mixture was stirred for 15 min before ammonium hydroxide (0.200 mL, 3.00 mmol) was added. The reaction was stirred at room temperature for 12 h. The reaction was added to water. The water was extracted 3 times with ethyl acetate. The combined organic layers were washed with brine, dried over magnesium sulfate, filtered, and concentrated. The residue was purified by silica gel column chromatography eluting with a gradient of 25%-65% ethyl acetate/heptane to obtain the title compound (15 mg, 25%) as a solid. ¹H NMR (DMSO-d₆) δ ppm 7.86 (m, 2H), 7.62 (d, 2H), 7.46 (m, 2H), 7.26 (d, 1H), 4.97 (m, 1H), 4.28 (m, 2H), 3.45 (dd, 1H), 3.23 (dd, 1H), 2.80 (m, 1H), 2.52 (s, 3H), 1.69-1.10 (m, 11H).

### Example 43

### (R)-6-(5-cyclopentyl-3-(3-methoxyphenyl)-4,5-dihydro-1H-pyrazol-1-yl)-2-methylnicotinonitrile

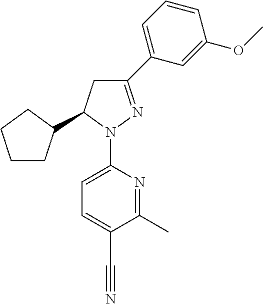

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 100 mg, 0.346 mmol) and 2-(3-methoxyphenyl)-4,4,5,5-tetramethyl-1,3,2-dioxaborolane (89.2 mg, 0.381 mmol). 48 mg of the title compound was isolated (38.3%) as a white/yellowish solid. ¹HNMR (DMSO-d₆, 500 MHz) δ ppm 7.83 (d, 1H), 7.41-7.36 (m, 3H), 7.22 (d, 1H), 7.03 (m, 1H), 4.95 (m, 1H), 3.83 (s, 3H), 3.45 (dd, 1H), 3.17 (dd, 1H), 2.79 (m, 1H), 2.51 (s, 3H), 1.72-1.32 (m, 7H), 1.11 (m, 1H).

### Example 44

### (R)-6-(1-(4-cyano-3-methylphenyl)-5-cyclopentyl-4,5-dihydro-1H-pyrazol-3-yl)-2-methoxynicotinamide

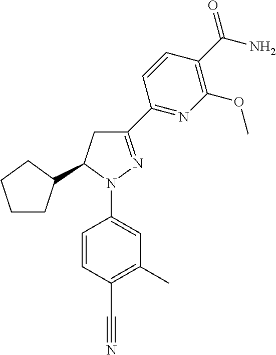

The title compound was prepared by the method used to prepare Example 11 from (R)-4-(3-chloro-5-cyclopentyl-4,5-dihydro-1H-pyrazol-1-yl)-2-methylbenzonitrile (Preparation 3, 174 mg, 0.605 mmol) and 2-methoxy-6-(4,4,5,5-tetramethyl-[1,3,2]dioxaborolan-2-yl)-nicotinamide (Preparation 21, 202 mg, 0.726 mmol). 35 mg of the title compound was isolated (14.5%) as a yellow solid. ¹H NMR (DMSO-d₆, 500 MHz) δ ppm 8.18 (d, 1H), 7.67 (m, 3H), 7.55 (d, 1H), 7.21 (d, 1H), 7.10 (dd, 1H), 4.86 (m, 1H), 4.01 (s, 3H), 3.45 (dd, 1H), 3.21 (dd, 1H), 2.47 (m, 1H), 2.41 (s, 3H), 1.73-1.01 (m, 8H).

### Example 45

### (R)-6-(5-cyclopentyl-3-(2-oxo-1,2,3,4-tetrahydroquinolin-6-yl)-4,5-dihydro-1H-pyrazol-1-yl)-2-methylnicotinonitrile

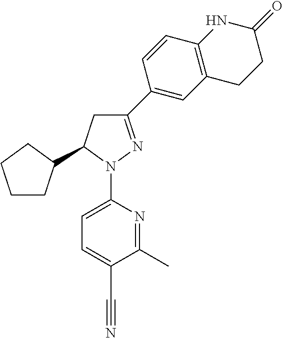

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 150 mg, 0.519 mmol) and 6-(4,4,5,5-tetramethyl-1,3,2-dioxaborolan-2-yl)-3,4-dihydroquinolin-2(1H)-one (Preparation 22, 170 mg, 0.623 mmol). 88 mg of the title compound was isolated (42%) as a solid. 1H NMR (400 MHz, DMSO-d₆) δ ppm 0.97-1.76 (m, 9H) 2.49 (s, 3H) 2.69-2.85 (m, 1H) 2.93 (t, J=7.61 Hz, 2H) 3.11 (dd, J=18.05, 4.00 Hz, 1H) 3.33-3.47 (m, 1H) 4.86-4.98 (m, 1H) 6.91 (d, J=8.19 Hz, 1H) 7.16 (d, J=8.78 Hz, 1H) 7.61 (dd, J=8.19, 1.95 Hz, 1H) 7.65 (d, J=1.56 Hz, 1H) 7.80 (d, J=8.97 Hz, 1H) 10.28 (s, 1H).

### Example 46

### (R)-6-(5-cyclopentyl-3-(3-oxo-3,4-dihydro-2H-benzo[b][1,4]oxazin-7-yl)-4,5-dihydro-1H-pyrazol-1-yl)-2-methylnicotinonitrile

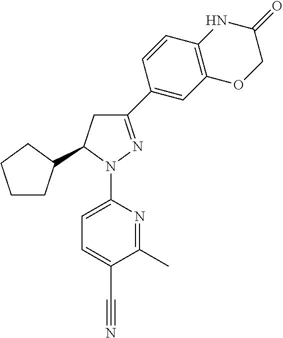

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 150 mg, 0.519 mmol) and 7-(4,4,5,5-tetramethyl-1,3,2-dioxaborolan-2-yl)-2H-benzo[b][1,4]oxazin-3(4H)-one (Preparation 23, 143 mg, 0.519 mmol). 37 mg of the title compound was isolated (18%) as a solid. 1H NMR (400 MHz, DMSO-d₆) δ ppm 0.97-1.76 (m, 11H) 2.69-2.86 (m, 1H) 3.11 (dd, J=18.05, 4.19 Hz, 1H) 3.33-3.46 (m, 1H) 4.62 (s, 2H) 4.85-4.97 (m, 1H) 6.95 (d, J=8.19 Hz, 1H) 7.16 (d, J=9.36 Hz, 1H) 7.33-7.47 (m, 2H) 7.80 (d, J=8.97 Hz, 1H) 10.91 (s, 1H).

### Example 47

### (R)-6-(5-cyclopentyl-3-(2-oxo-1,2,3,4-tetrahydroquinazolin-6-yl)-4,5-dihydro-1H-pyrazol-1-yl)-2-methylnicotinonitrile

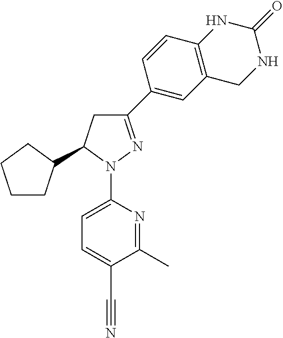

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 150 mg, 0.519 mmol) and 6-(4,4,5,5-tetramethyl-1,3,2-dioxaborolan-2-yl)-3,4-dihydroquinazolin-2(1H)-one (Preparation 24, 143 mg, 0.519 mmol). 57 mg of the title compound was isolated (27%) as a solid. 1H NMR (400 MHz, DMSO-d₆) δ ppm 0.97-1.76 (m, 9H) 2.50 (s, 3H) 2.69-2.87 (m, 1H) 3.08 (dd, J=17.95, 4.10 Hz, 1H) 3.39 (dd, J=17.95, 11.32 Hz, 1H) 4.37 (s, 2H) 4.83-4.96 (m, 1H) 6.83 (d, J=8.78 Hz, 1H) 6.92 (s, 1H) 7.15 (d, J=8.97 Hz, 1H) 7.54-7.64 (m, 1H) 7.80 (d, J=8.78 Hz, 1H) 9.27 (s, 1H).

### Example 48

### (R)-ethyl 6-(1-(4-cyano-3-methylphenyl)-5-cyclopentyl-4,5-dihydro-1H-pyrazol-3-yl)-2-ethoxynicotinate

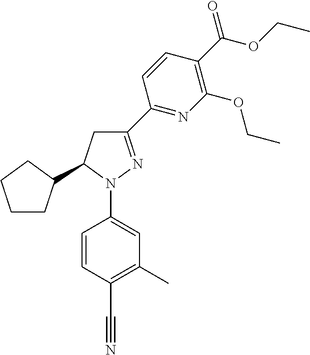

The title compound was prepared by the method used to prepare Example 11 from (R)-4-(3-chloro-5-cyclopentyl-4,5-dihydro-1H-pyrazol-1-yl)-2-methylbenzonitrile (Preparation 3, 374 mg, 1.30 mmol) and ethyl 2-ethoxy-6-(4,4,5,5-tetramethyl-1,3,2-dioxaborolan-2-yl)nicotinate (Preparation 30, 500 mg, 1.56 mmol). 507 mg of the title compound was isolated (88%) as a gum. 1H NMR (500 MHz, DMSO-d₆) δ ppm 1.05 (1 H, m), 1.25 (1H, d, J=10.0 Hz), 1.30 (3H, t, J=7.1 Hz), 1.36 (3H, t, J=7.1 Hz), 1.51 (1 H, m), 1.58 (1H, d, J=7.8 Hz), 1.77 (1H, m), 2.44 (3H, s), 2.52 (1H, d, J=3.7 Hz), 3.19 (1H, dd, J=18.4, 4.0 Hz), 3.32 (3H, s), 3.46 (1H, dd, J=18.4, 11.8 Hz), 4.27 (2H, q, J=7.1 Hz), 4.46 (2H, m, J=7.0, 6.7, 6.6, 6.6 Hz), 4.89 (1H, dt, J=11.7, 4.0 Hz), 7.14 (1 H, dd, J=8.7, 1.8 Hz), 7.24 (1H, s), 7.59 (1H, d, J=8.8 Hz), 7.68 (1H, d, J=8.1 Hz), 8.13 (1H, d, J=7.8 Hz).

### Example 49

### (R)-6-(1-(4-cyano-3-methylphenyl)-5-cyclopentyl-4,5-dihydro-1H-pyrazol-3-yl)-2-ethoxynicotinic acid

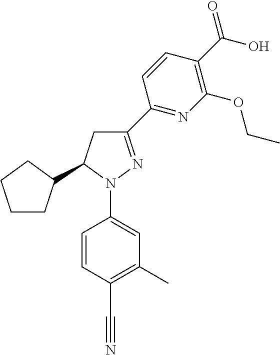

The title compound was prepared by the method used to prepare Example 33 from (R)-ethyl 6-(1-(4-cyano-3-methylphenyl)-5-cyclopentyl-4,5-dihydro-1H-pyrazol-3-yl)-2-ethoxynicotinate (Example 48, 500 mg, 1.12 mmol). 155 mg of the title compound was isolated (98%) as a yellow solid. 1H NMR (500 MHz, DMSO-d₆) δ ppm 1.24 (1H, br. s.), 1.33 (1H, br. s.), 1.36 (3H, t, J=7.0 Hz), 1.51 (1H, m), 1.51 (1H, d, J=7.3 Hz), 1.58 (1H, br. s.), 1.76 (1H, d, J=2.9 Hz), 2.44 (2H, s), 2.53 (1H, d, J=4.1 Hz), 3.19 (1H, dd, J=18.3, 3.9 Hz), 3.32 (3H, s.), 3.46 (1H, dd, J=18.4, 11.8 Hz), 4.46 (2H, m, J=10.6, 7.1, 7.1, 3.6, 3.6 Hz), 4.89 (1H, dt, J=11.6, 4.1 Hz), 7.13 (1H, dd, J=8.8, 2.2 Hz), 7.24 (1H, d, J=1.7 Hz), 7.59 (1H, d, J=8.8 Hz), 7.67 (1H, d, J=7.8 Hz), 8.12 (1H, d, J=7.8 Hz), 12.85 (1H, s).

### Example 50

### (R)-ethyl 6-(1-(5-cyano-6-methylpyridin-2-yl)-5-cyclopentyl-4,5-dihydro-1H-pyrazol-3-yl)-2-ethoxynicotinate

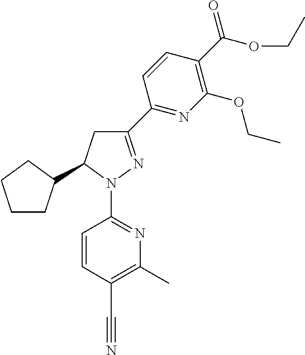

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 375 mg, 1.3 mmol) and ethyl 2-ethoxy-6-(4,4,5,5-tetramethyl-1,3,2-dioxaborolan-2-yl)nicotinate (Preparation 30, 500 mg, 1.56 mmol). 39 mg of the title compound was isolated (5.6%) as a solid. 1H NMR (400 MHz, DMSO-d₆) δ ppm 1.09 (2H, t, J=7.0 Hz), 1.31 (3H, t, J=7.1 Hz), 1.36 (5H, t, J=7.0 Hz), 1.52 (2H, br. s.), 1.69 (1H, br. s.), 2.54 (3H, s), 2.82 (1H, br. s.), 3.17 (1H, dd, J=18.6, 4.6 Hz), 3.40 (1H, m), 3.49 (1H, dd, J=18.6, 11.6 Hz), 4.28 (2H, q, J=7.1 Hz), 4.46 (2H, m, J=10.7, 7.1, 7.1, 3.7, 3.6 Hz), 5.00 (1H, ddd, J=11.6, 4.7, 4.6 Hz), 7.28 (1H, s), 7.71 (1H, d, J=7.8 Hz), 7.90 (1H, d, J=8.8 Hz), 8.17 (1H, s).

### Example 51

### (R)-6-(1-(5-cyano-6-methylpyridin-2-yl)-5-cyclopentyl-4,5-dihydro-1H-pyrazol-3-yl)-2-ethoxynicotinic acid

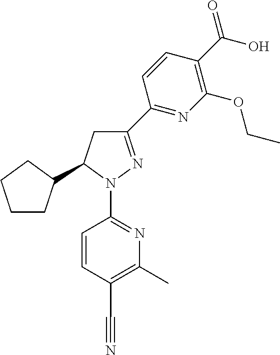

The title compound was prepared by the method used to prepare Example 33 from (R)-ethyl 6-(1-(5-cyano-6-methylpyridin-2-yl)-5-cyclopentyl-4,5-dihydro-1H-pyrazol-3-yl)-2-ethoxynicotinate (Example 50, 500 mg, 1.12 mmol). 460 mg of the title compound was isolated (98%) as a yellow solid. 1H NMR (400 MHz, DMSO-d₆) δ ppm 1.09 (1H, t, J=7.1 Hz), 1.36 (3H, t, J=7.1 Hz), 1.52 (2H, br. s.), 1.60 (1H, d, J=4.6 Hz), 1.70 (1H, d, J=7.3 Hz), 2.54 (3H, s), 2.82 (1H, br. s.), 3.17 (1H, dd, J=18.5, 4.6 Hz), 3.32 (3H, s), 3.49 (1H, dd, J=18.5, 11.7 Hz), 4.47 (2H, m, J=10.6, 7.1, 7.1, 3.7, 3.5 Hz), 5.00 (1 H, dt, J=11.5, 4.6 Hz), 7.26 (1H, d, J=8.8 Hz), 7.69 (1H, d, J=7.8 Hz), 7.90 (1H, d, J=8.8 Hz), 8.15 (1H, d, J=7.8 Hz), 12.95 (1H, s).

### Example 52

### (R)-6-(5-cyclopentyl-3-(4-hydroxy-3-methoxyphenyl)-4,5-dihydro-1H-pyrazol-1-yl)-2-methylnicotinonitrile

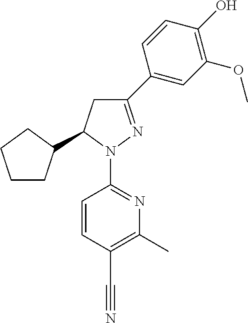

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 116 mg, 0.40 mmol) and 2-methoxy-4-(4,4,5,5-tetramethyl-1,3,2-dioxaborolan-2-yl)phenol (100 mg, 0.40 mmol). 92 mg of the title compound was isolated (61%) as a solid. 1H NMR (400 MHz, DMSO-d₆) δ ppm 0.97-1.76 (m, 10H) 2.77 (br. s., 1H) 3.11 (dd, J=17.95, 4.29 Hz, 1H) 3.33-3.46 (m, 1H) 3.85 (s, 3H) 4.84-4.95 (m, 1H) 6.84 (d, J=8.19 Hz, 1H) 7.17 (d, J=8.39 Hz, 1H) 7.22 (dd, J=8.19, 1.95 Hz, 1H) 7.39 (d, J=1.95 Hz, 1H) 7.79 (d, J=8.97 Hz, 1H) 9.54 (s, 1H).

### Example 53

### (R)-methyl 6-(1-(5-cyano-6-methylpyridin-2-yl)-5-cyclopentyl-4,5-dihydro-1H-pyrazol-3-yl)-2-ethoxynicotinate

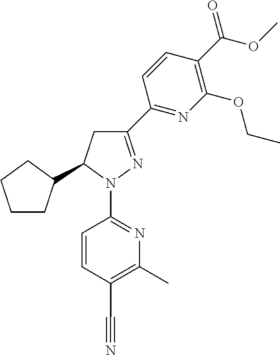

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 100 mg, 0.346 mmol) and methyl 2-ethoxy-6-(4,4,5,5-tetramethyl-1,3,2-dioxaborolan-2-yl)nicotinate (Preparation 31, 196 mg, 0.415 mmol). 30 mg of the title compound was isolated (20%) as a solid. 1H NMR (500 MHz, DMSO-d₆) δ ppm 1.24 (1 H, br. s.), 1.33 (1H, br. s.), 1.36 (3H, t, J=7.0 Hz), 1.51 (1H, m), 1.51 (1H, d, J=7.3 Hz), 1.58 (1H, br. s.), 1.76 (1H, d, J=2.9 Hz), 2.44 (2H, s), 2.53 (1H, d, J=4.1 Hz), 3.19 (1H, dd, J=18.3, 3.9 Hz), 3.32 (3H, br. s.), 3.46 (1H, dd, J=18.4, 11.8 Hz), 3.88 (3 H, s), 4.46 (2H, m, J=10.6, 7.1, 7.0, 3.7, 3.6 Hz), 4.89 (1H, ddd, J=11.6, 4.1, 4.0 Hz), 7.13 (1H, dd, J=8.8, 2.2 Hz), 7.59 (1H, d, J=8.8 Hz), 7.67 (1H, d, J=7.8 Hz), 8.12 (1H, d, J=7.8 Hz).

### Example 54

### (R)-2-(4-(1-(5-cyano-6-methylpyridin-2-yl)-5-cyclopentyl-4,5-dihydro-1H-pyrazol-3-yl)-2-methoxyphenyl)acetic acid

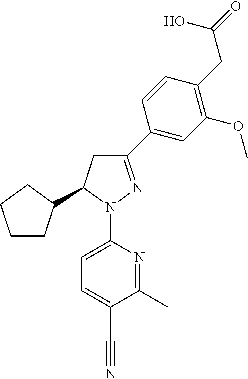

1,1′-Bis(diphenylphospino)ferrocene-palladium dichloride (45.5 mg, 0.061 mmol), 2-(4-bromo-2-methoxyphenyl)acetic acid (Preparation 26, 300 mg, 1.22 mmol), bis(pinacolato)diboron (373 mg, 1.47 mmol), potassium acetate (372 mg, 3.67 mmol) were combined in a microwave vial with degassed 1,4-dioxane (3 mL). The vial was sealed and heated at 100° C. for 60 min in a microwave reactor. After cooling to room temperature, the reaction mixture was diluted with ethyl acetate (150 mL), filtered through celite and extracted with water (50 mL). The organic layer was dried over magnesium sulfate, filtered, and concentrated to give 2-(2-methoxy-4-(4,4,5,5-tetramethyl-1,3,2-dioxaborolan-2-yl)phenyl)acetic acid and the intermediate was carried on without further purification. The crude 2-(2-methoxy-4-(4,4,5,5-tetramethyl-1,3,2-dioxaborolan-2-yl)phenyl)acetic acid (267 mg, 0.914 mmol), (R)-6-(3-chloro-5-cyclopentyl-4,5-dihydro-1H-pyrazol-1-yl)-2-methylnicotinonitrile (Preparation 7, 220 mg, 0.762 mmol), and tetrakis(triphenylphosphine) palladium (44.8 mg, 0.038 mmol) were combined in 1,2-dimethoxyethane (5 mL). 2M aqueous sodium carbonate solution (0.838 mL, 178 mg, 1.68 mmol) was added and reaction mixture was heated at 88° C. for 16h. The reaction was cooled to room temperature, diluted with water (100 mL) and acidified to pH 2 by the addition of 1N aqueous hydrochloric acid. The mixture was extracted with ethyl acetate (150 mL), dried over magnesium sulfate and filtered through celite give an orange oil. This material was purified by silica gel chromatography eluting with a gradient of 0-100% ethyl acetate/heptane to obtain an impure product. To the product obtained was added 1N aqueous sodium hydroxide (10 mL) and the mixture was extracted 2 times with diethyl ether (20 mL). The aqueous layer was then acidified to pH 2 by addition of 1N aqueous hydrochloric acid and extracted with ethyl acetate (100 mL). The organic phase was dried over magnesium sulfate, filtered and concentrated to give the title compound (6 mg, 2%) as a yellow gum. 1H NMR (400 MHz, CHLOROFORM-d) δ ppm 1.31-1.69 (m, 6H) 1.75 (br. s., 2H) 2.59 (s, 3H) 2.82 (br. s., 1H) 2.99 (dd, J=17.36, 4.29 Hz, 1H) 3.32 (dd, J=17.46, 11.41 Hz, 1H) 3.63-3.73 (m, 2H) 3.91 (s, 3H) 4.92-5.06 (m, 1H) 7.12-7.23 (m, 3H) 7.39 (d, J=1.37 Hz, 1H) 7.56 (d, J=8.78 Hz, 1H).

### Example 55

### (R)-6-(1-(4-cyano-3-methylphenyl)-5-cyclopentyl-4,5-dihydro-1H-pyrazol-3-yl)-2-ethoxynicotinamide

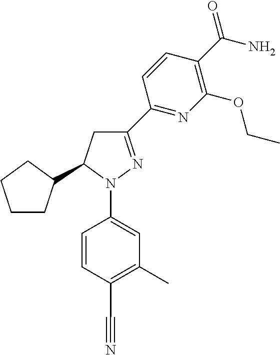

The title compound was prepared by the method used to prepare Example 42 from (R)-6-(1-(4-cyano-3-methylphenyl)-5-cyclopentyl-4,5-dihydro-1H-pyrazol-3-yl)-2-ethoxynicotinic acid (Example 49, 0.15 g, 0.358 mmol) and ammonium hydroxide (0.500 mL, 0.940 g, 7.51 mmol). 53.8 mg of the title compound was isolated (36%) as a yellow solid. 1H NMR (500 MHz, DMSO-d₆) δ ppm 1.04 (1H, br. s.), 1.24 (3H, br. s.), 1.41 (3H, t, J=7.0 Hz), 1.51 (2H, d, J=8.1 Hz), 1.58 (1H, br. s.), 1.77 (1H, d, J=8.1 Hz), 2.44 (2H, s), 2.52 (1H, d, J=2.0 Hz), 3.20 (1H, dd, J=18.3, 3.9 Hz), 3.32 (3H, s), 3.47 (1H, dd, J=18.4, 11.8 Hz), 4.54 (2H, m, J=10.6, 7.1, 7.1, 3.6, 3.6 Hz), 4.89 (1H, dt, J=11.9, 4.1 Hz), 7.13 (1H, dd, J=8.7, 2.1 Hz), 7.24 (1H, d, J=1.7 Hz), 7.59 (1H, d, J=8.8 Hz), 7.72 (1H, d, J=7.8 Hz), 8.23 (1H, d, J=7.8 Hz).

### Example 56

### (R)-6-(1-(4-cyano-3-methylphenyl)-5-cyclopentyl-4,5-dihydro-1H-pyrazol-3-yl)-2-methoxy-N-methylnicotinamide

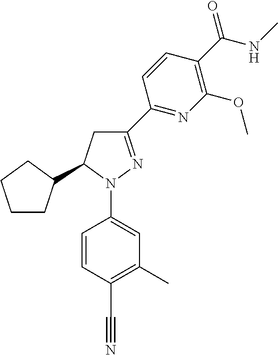

The title compound was prepared by the method used to prepare Example 42 from (R)-6-(1-(4-cyano-3-methylphenyl)-5-cyclopentyl-4,5-dihydro-1H-pyrazol-3-yl)-2-methoxynicotinic acid (Example 4, 0.10 g, 0.247 mmol) and 2M methylamine in tetrahydrofuran (1.24 mL, 0.0767 g, 2.47 mmol). 94.3 mg of the title compound was isolated (92%) as a yellow solid. 1H NMR (500 MHz, DMSO-d₆) δ ppm 1.05 (1H, dd, J=12.2, 9.5 Hz), 1.33 (1H, m), 1.44 (1H, m), 1.50 (2H, m), 1.77 (1H, m), 2.44 (3H, s), 2.52 (3H, d, J=7.6 Hz), 2.82 (3H, d, J=4.9 Hz), 3.22 (1H, dd, J=18.4, 4.0 Hz), 3.48 (1

H, dd, J=18.3, 11.7 Hz), 4.04 (3H, s), 4.89 (1H, dt, J=11.6, 4.1 Hz), 7.13 (1H, dd, J=8.7, 2.1 Hz), 7.24 (1H, d, J=1.7 Hz), 7.59 (1H, d, J=8.8 Hz), 7.73 (1H, d, J=8.8 Hz), 8.20 (1H, d, J=7.8 Hz), 8.22 (1H, m).

### Example 57

### (R)-6-(5-cyclopentyl-3-(4-hydroxy-3,5-dimethylphenyl)-4,5-dihydro-1H-pyrazol-1-yl)-2-methylnicotinonitrile

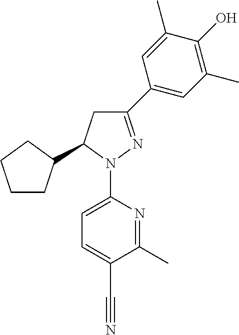

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 175 mg, 0.605 mmol) and 2,6-dimethyl-4-(4,4,5,5-tetramethyl-1,3,2-dioxaborolan-2-yl)phenol (150 mg, 0.605 mmol). 147 mg of the title compound was isolated (65%) as a solid. 1H NMR (400 MHz, CHLOROFORM-d) δ ppm 1.03-1.87 (m, 8H) 2.31 (s, 6H) 2.59 (s, 3H) 2.77-2.90 (m, 1H) 2.99 (dd, J=17.46, 4.19 Hz, 1H) 3.30 (dd, J=17.36, 11.12 Hz, 1H) 4.93-5.03 (m, 1H) 7.21 (d, J=8.39 Hz, 1H) 7.42 (s, 2H) 7.56 (d, J=8.78 Hz, 1H).

### Example 58

### (R)-6-(1-(5-cyano-6-methylpyridin-2-yl)-5-cyclopentyl-4,5-dihydro-1H-pyrazol-3-yl)-2-methoxy-N-methylnicotinamide

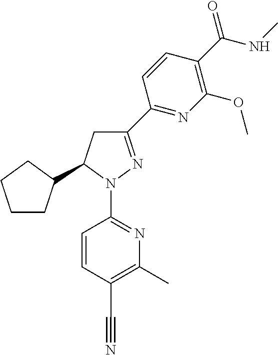

The title compound was prepared by the method used to prepare Example 42 from (R)-6-(1-(5-cyano-6-methylpyridin-2-yl)-5-cyclopentyl-4,5-dihydro-1H-pyrazol-3-yl)-2-methoxynicotinic acid (Example 33, 0.10 g, 0.247 mmol) and 2M methylamine in tetrahydrofuran (1.23 mL, 0.0766 g, 2.47 mmol). 96.8 mg of the title compound was isolated (62%) as a yellow solid. 1H NMR (500 MHz, DMSO-d₆) δ ppm 1.05 (1H, dd, J=12.2, 9.5 Hz), 1.33 (1H, m), 1.44 (1H, m), 1.50 (2H, m), 1.77 (1H, m), 2.44 (3H, s), 2.52 (3H, d, J=7.6 Hz), 2.82 (3H, d, J=4.9 Hz), 3.22 (1H, dd, J=18.4, 4.0 Hz), 3.48 (1 H, dd, J=18.3, 11.7 Hz), 4.04 (3H, s), 4.89 (1H, dt, J=11.6, 4.1 Hz), 7.13 (1H, dd, J=8.7, 2.1 Hz), 7.26 (1H, d, J=1.7 Hz), 7.75 (1H, d, J=8.8 Hz), 8.20 (1H, d, J=7.8 Hz), 8.22 (1H, m).

### Example 59

### (R)-6-(1-(4-cyano-3-methylphenyl)-5-cyclopentyl-4,5-dihydro-1H-pyrazol-3-yl)-2-methoxy-N,N-dimethylnicotinamide

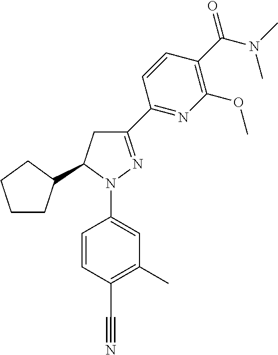

The title compound was prepared by the method used to prepare Example 42 from (R)-6-(1-(4-cyano-3-methylphenyl)-5-cyclopentyl-4,5-dihydro-1H-pyrazol-3-yl)-2-methoxynicotinic acid (Example 4, 0.048 g, 0.12 mmol) and 2M dimethylamine in tetrahydrofuran (0.595 mL, 0.0542 g, 1.19 mmol). 36.3 mg of the title compound was isolated (71%) as a yellow solid. 1H NMR (500 MHz, DMSO-d₆) δ ppm 1.35 (2H, s), 1.51 (2H, br. s.), 1.77 (1H, br. s.), 2.44 (3H, s), 2.52 (1H, br. s.), 2.81 (3H, s), 2.98 (3 H, s), 3.22 (1H, dd, J=18.4, 4.0 Hz), 3.32 (3H, s), 3.48 (1H, dd, J=18.4, 11.6 Hz), 3.96 (3H, s), 4.87 (1H, d, J=11.7 Hz), 7.12 (1H, d, J=2.0 Hz), 7.22 (1H, d, J=2.0 Hz), 7.58 (1H, d, J=8.5 Hz), 7.69 (2H, s).

### Example 60

### (R)-6-(1-(5-cyano-6-methylpyridin-2-yl)-5-cyclopentyl-4,5-dihydro-1H-pyrazol-3-yl)-2-methoxy-N,N-dimethylnicotinamide

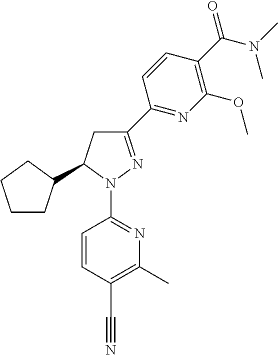

The title compound was prepared by the method used to prepare Example 42 from (R)-6-(1-(5-cyano-6-methylpyridin-2-yl)-5-cyclopentyl-4,5-dihydro-1H-pyrazol-3-yl)-2-methoxynicotinic acid (Example 33, 0.10 g, 0.247 mmol) and 2M dimethylamine in tetrahydrofuran (1.24 mL, 0.112 g, 2.47 mmol). 81 mg of the title compound was isolated (76%) as a white solid. 1H NMR (500 MHz, DMSO-d₆) δ ppm 1.17 (1H, m), 1.34 (1H, d, J=12.2 Hz), 1.43 (1H, m), 1.42 (1H, d, J=7.1 Hz), 1.53 (2H, d, J=4.4 Hz), 1.70 (1H, d, J=7.6 Hz), 2.54 (3H, s), 2.80 (3H, s), 2.98 (3H, s), 3.32 (3H, s), 3.51 (1 H, dd, J=18.7, 11.6 Hz), 3.96 (3H, s), 4.99 (1H, ddd, J=11.4, 4.7, 4.6 Hz), 7.25 (1H, d, J=9.0 Hz), 7.72 (2H, d, J=2.4 Hz), 8.21 (1H, d, J=9.0 Hz).

### Example 61

### (R)-6-(5-cyclopentyl-3-(3,5-difluoro-4-hydroxyphenyl)-4,5-dihydropyrazol-1-yl)-2-methylnicotinonitrile

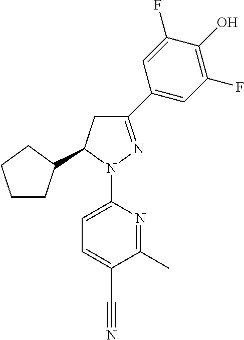

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 104 mg, 0.360 mmol) and tert-butyl(2,6-difluoro-4-(4,4,5,5-tetramethyl-1,3,2-dioxaborolan-2-yl)phenoxy)dimethylsilane (WO 2008/063300, 133 mg, 0.360 mmol). 60 mg of the title compound was isolated (43%) as a solid. 1H NMR (400 MHz, DMSO-d₆) δ ppm 1.01-1.77 (m, 8H) 2.50 (s, 3H) 2.74-2.90 (m, 1H) 3.01 (dd, J=17.85, 4.39 Hz, 1H) 3.27-3.35 (m, 1H) 4.89-5.01 (m, 1H) 7.16 (d, J=8.78 Hz, 1H) 7.32 (d, J=9.56 Hz, 2H) 7.61 (d, J=8.97 Hz, 1H).

### Example 62

### (R)-6-(5-cyclopentyl-3-(3-fluoro-4-hydroxyphenyl)-4,5-dihydropyrazol-1-yl)-2-methylnicotinonitrile

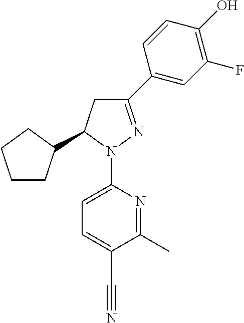

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 185 mg, 0.641 mmol) and 3-fluoro-4-hydroxyphenyl boronic acid (100 mg, 0.641 mmol). 167 mg of the title compound was isolated (71%) as a solid. 1H NMR (400 MHz, DMSO-d₆) δ ppm 0.95-1.76 (m, 8H) 2.50 (s, 3H) 2.66-2.85 (m, 1H) 3.11 (dd, J=18.05, 4.19 Hz, 1H) 3.30-3.44 (m, 1H) 4.81-4.99 (m, 1H) 6.99-7.07 (m, 1H) 7.17 (dd, J=8.88, 0.49 Hz, 1H) 7.39-7.52 (m, 1H) 7.61 (dd, J=12.39, 2.05 Hz, 1 H) 7.79 (d, J=8.78 Hz, 1H) 10.43 (br. s., 1H).

### Example 63

### (R)-6-(1-(4-cyano-3-methylphenyl)-5-cyclopentyl-4,5-dihydro-1H-pyrazol-3-yl)-2-methoxy-N-(1H-tetrazol-5-yl)nicotinamide

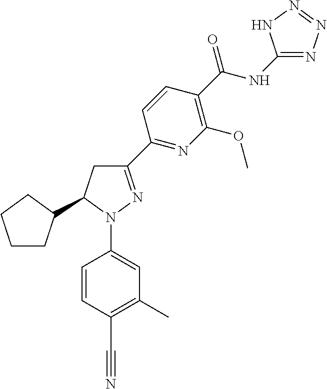

The title compound was prepared by the method used to prepare Example 42 from (R)-6-(1-(4-cyano-3-methylphenyl)-5-cyclopentyl-4,5-dihydro-1H-pyrazol-3-yl)-2-methoxynicotinic acid (Example 4, 90 mg, 0.220 mmol) and 5-amino-tetrazole (28.5 mg, 0.335 mmol). 20 mg of the title compound was isolated (19%) as a yellow solid. ¹H NMR (DMSO-d₆) δ ppm 11.85 (s, 1H), 8.16 (d, 1H), 7.78 (d, 1H), 7.61 (d, 1H), 7.27 (s, 1H), 7.16 (d, 1H), 4.93 (m, 1H), 4.06 (s, 3H), 3.50 (dd, 1H), 3.27 (dd, 1H), 2.50 (s, 3H), 1.78-1.05 (m, 8H).

### Example 64

### (R)-6-(5-cyclopentyl-3-(4,4-dimethyl-2-oxo-2,4-dihydro-1H-benzo[d][1,3]oxazin-6-yl)-4,5-dihydro-1H-pyrazol-1-yl)-2-methylnicotinonitrile

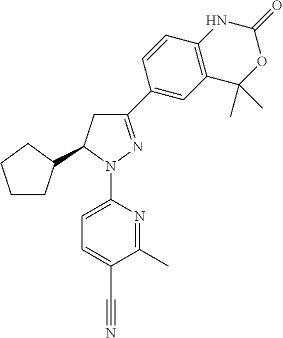

The title compound was prepared by the method used to prepare Example 11 from (R)-6-(3-chloro-5-cyclopentyl-4,5-dihydro-1H-pyrazol-1-yl)-2-methylnicotinonitrile (Preparation 7, 150 mg, 0.519 mmol) and 4,4-dimethyl-6-(4,4,5,5-tetramethyl-1,3,2-dioxaborolane-2-yl)-1H-benzo[d][1,3]oxazin-2(4H)-one (Preparation 25, 157 mg, 0.519 mmol). 148 mg of the title compound was isolated (66%) as a solid. 1H NMR (400 MHz, CHLOROFORM-d) δ ppm 1.05-1.21 (m, 1H) 1.21-1.41 (m, 3H) 1.43-1.73 (m, 4H) 1.80 (d, J=2.93 Hz, 6H) 2.61 (s, 3H) 2.79-2.92 (m, 1H) 3.00 (dd, J=17.36, 4.49 Hz, 1H) 3.33 (dd, J=17.36, 11.51 Hz, 1H) 4.97-5.08 (m, 1H) 6.89 (d, J=8.19 Hz, 1H) 7.22 (dd, J=8.78, 0.59 Hz, 1H) 7.55 (dd, J=8.29, 1.85 Hz, 1H) 7.59 (d, J=8.78 Hz, 1H) 7.63 (d, J=1.76 Hz, 1H) 8.65 (s, 1H).

### Example 65

### (R)-6-(1-(4-cyano-3-methylphenyl)-5-cyclopentyl-4,5-dihydro-1H-pyrazol-3-yl)-2-methoxy-N-(methylsulfonyl)nicotinamide

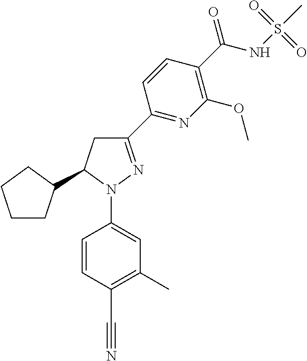

The title compound was prepared by the method used to prepare Example 42 from (R)-6-(1-(4-cyano-3-methylphenyl)-5-cyclopentyl-4,5-dihydro-1H-pyrazol-3-yl)-2-methoxynicotinic acid (Example 4, 100 mg, 0.247 mmol) and methanesulfonamide (36.3 mg, 0.370 mmol). 35 mg of the title compound was isolated as a yellow solid (29%). ¹H NMR (DMSO-d₆) δ ppm 11.60 (s, 1H), 5.05 (d, 1H), 7.74 (d, 1H), 7.60 (d, 1H), 7.26 (s, 1H), 7.15 (d, 1H), 4.91 (m, 1H), 4.02 (s, 3H), 3.48 (dd, 1H), 3.37 (s, 3H), 3.20 (dd, 1H), 2.50 (m, 1H), 2.45 (s, 3H), 1.79-1.02 (m, 8H).

### Example 66

### (R)—N-(6-(1-(5-cyano-6-methylpyridin-2-yl)-5-cyclopentyl-4,5-dihydro-1H-pyrazol-3-yl)pyridin-2-yl)acetamide

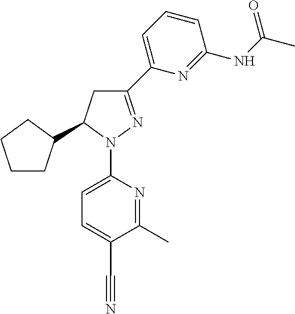

A mixture of [1,1′-bis(diphenylphosphino)ferrocene]palladium (II) chloride (62.7 mg, 0.084 mmol), potassium acetate (248 mg, 2.53 mmol), and bis(pinacolato)diborane (236 mg, 0.928 mmol) was purged with nitrogen. 1,2-dimethoxyethane (3 mL) and N-(6-chloropyridin-2-yl)acetamide (Preparation 27, 144 mg, 0.844 mmol) were added. The reaction vessel was sealed and the mixture was heated to 80° C. for 16 h. The mixture was cooled to room temperature and (R)-6-(3-chloro-5-cyclopentyl-4,5-dihydro-1H-pyrazol-1-yl)-2-methylnicotinonitrile (Preparation 7, 214 mg, 0.740 mmol), tetrakis(triphenylphosphine) palladium (42.8 mg, 0.037 mmol) and 2M aqueous sodium carbonate (0.925 mL, 1.84 mmol) were added. The reaction was stirred at 80° C. for 16 h. The mixture was filtered through celite. The filtrate was diluted with ethyl acetate and water. The layers were separated. The organic layer was washed with brine, dried over magnesium sulfate, filtered and concentrated. The residue was purified by silica gel column chromatography eluting with a gradient of 0%-50% ethyl acetate/heptane. The fractions containing product were concentrated and triturated with diethyl ether, yielding title compound (12 mg, 4%) as a solid. ¹HNMR (DMSO-d₆, 500 MHz) δ ppm 10.47 (s, 1H), 8.08 (m, 1H), 7.86 (m, 2H), 7.75 (d, 1H), 7.24 (d, 1H), 4.97 (m, 1H), 3.48 (dd, 1H), 3.18 (dd, 1H), 2.83 (m, 1H), 2.53 (s, 3H), 2.13 (s, 3H), 1.73-1.12 (m, 8H).

### Example 67

### (R)-6-(5-cyclopentyl-3-(6-isopropoxypyridin-2-yl)-4,5-dihydro-1H-pyrazol-1-yl)-2-methylnicotinonitrile

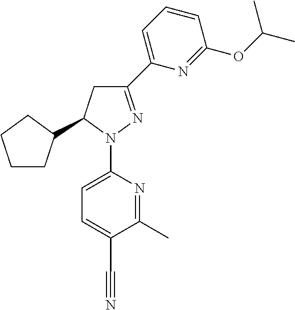

A mixture of [1,1′-bis(diphenylphosphino)ferrocene]palladium (II) chloride (134 mg, 0.179 mmol), potassium acetate (527 mg, 5.37 mmol), and bis(pinacolato)diborane (500 mg, 1.97 mmol) was purged with nitrogen. 1,2-dimethoxyethane (5 mL) and 2-chloro-6-isopropoxypyridine (Preparation 28, 307 mg, 1.79 mmol) were added. The reaction vessel was sealed and the mixture was heated to 80° C. for 16 h. The mixture was cooled to room temperature and filtered through celite and ethyl acetate was added. The filtrate was partitioned with water, separated, washed with brine, dried over magnesium sulfate, filtered, and concentrated. To the residue were added (R)-6-(3-chloro-5-cyclopentyl-4,5-dihydro-1H-pyrazol-1-yl)-2-methylnicotinonitrile (Preparation 7, 470 mg, 1.63 mmol), tetrakis(triphenylphosphine) palladium (93.7 mg, 0.0810 mmol) and 2M aqueous sodium carbonate (2.04 mL, 4.07 mmol) and 1,2-dimethoxyethane (10 mL). The reaction was stirred at 80° C. for 16 h. The reaction mixture was cooled to room temperature, filtered through celite and ethyl acetate was added. The filtrate was partitioned with water, separated, washed with brine, dried over magnesium sulfate, filtered, and concentrated. The residue was purified by silica gel column chromatography (ethyl acetate/heptane) to yield the title compound as a white solid (66 mg, 72%). ¹HNMR (DMSO-d₆, 500 MHz) δ ppm 7.86 (d, 1H), 7.75 (t, 1H), 7.62 (d, 1H), 7.21 (d, 1H), 6.78 (d, 1H), 5.27 (m, 1H), 4.96 (m, 1H), 3.47 (dd, 1H), 3.15 (dd, 1H), 2.81 (m, 1H), 2.53 (s, 3H), 1.69 (m, 1H), 1.62 (m, 1H), 1.52 (m, 2H), 1.41 (m, 2H), 1.33 (dd, 6H), 1.31 (m, 1H), 1.12 (m, 1H).

### Example 68

### (R)-6-(1-(5-cyano-6-methylpyridin-2-yl)-5-cyclopentyl-4,5-dihydro-1H-pyrazol-3-yl)-2-methoxy-N-(methylsulfonyl)nicotinamide

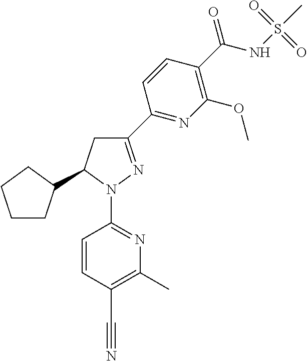

(R)-6-(1-(5-cyano-6-methylpyridin-2-yl)-5-cyclopentyl-4,5-dihydro-1H-pyrazol-3-yl)-2-methoxynicotinic acid (Example 33, 80 mg, 0.197 mmol), methanesulfonamide (19.3 mg, 0.197 mmol), N′N′-diisopropylethylamine (102 mg, 0.788 mmol) and N,N,N′N′-tetramethyl-O-(7-azabenzotriazol-1-yl)uronium hexafluorophosphate (74.9 mg, 0.197 mmol) were combined in dichloromethane (2 mL) and stirred at room temperature 16 h. The reaction was diluted with dichloromethane and extracted with water. The organic layer was washed with 0.1N aqueous hydrochloric acid and brine, dried over magnesium sulfate, filtered and concentrated. The residue was purified by column chromatography using a gradient 20%-50% ethyl acetate/heptane to give the title compound (45 mg, 47%) as a yellow solid. ¹H NMR (DMSO-d₆, 500 MHz) δ ppm 11.7 (s, 1H), 8.06 (d, 1H), 7.91 (d, 1H), 7.76 (d, 1H), 7.28 (d, 1H), 5.03 (m, 1H), 4.02 (s, 3H), 3.52 (dd, 1H), 3.37 (s, 3H), 3.19 (dd, 1H), 2.84 (m, 1H), 2.54 (s, 3H), 1.72-1.09 (m, 8H).

### Example 69

### Methyl 6-(1-(4-cyano-3-methylphenyl)-5-(3,3-difluorocyclobutyl)-4,5-dihydro-1H-pyrazol-3-yl)-2-methoxynicotinate

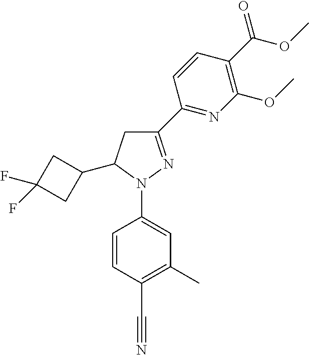

The title compound was prepared by the method used for Example 2, Method 2 from 4-(5-(3,3-difluorocyclobutyl)-3-oxopyrazolidin-1-yl)-2-methylbenzonitrile (Preparation 32, 131 mg). 128 mg isolated (69%) as a yellow solid. 1H NMR (400 MHz, CHLOROFORM-d) δ ppm 2.25-2.83 (5H, m), 2.54 (3H, s), 3.35 (1H, dd, J=18.4, 4.0 Hz), 3.56 (1H, dd, J=11.5, 20 Hz), 3.93 (3H, s), 4.09 (3H, s), 4.70 (1H, m), 7.00 (1H, dd, J=8.6, 2.3 Hz), 7.11 (1H, d, J=2.1 Hz), 7.51 (1H, d, J=8.6 Hz), 7.71 (1H, d, J=7.8 Hz), 8.20 (1H, d, J=8Hz).

### Example 70

### 6-(1-(4-cyano-3-methylphenyl)-5-(3,3-difluorocyclobutyl)-4,5-dihydro-1H-pyrazol-3-yl)-2-methoxynicotinic acid

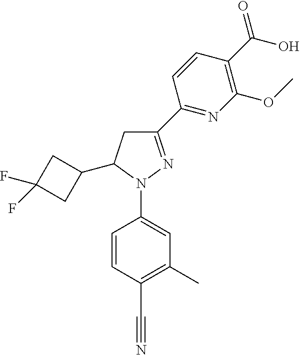

To a solution of methyl 6-(1-(4-cyano-3-methylphenyl)-5-(3,3-difluorocyclobutyl)-4,5-dihydro-1H-pyrazol-3-yl)-2-methoxynicotinate (Example 69, 54 mg, 0.12 mmol) in tetrahydrofuran (2 mL) was added 1M aqueous sodium hydroxide (1.2 mL, 1.2 mmol) The reaction was stirred at room temperature for 16 h. The reaction was acidified to pH 4 by addition of 1M aqueous hydrochloric acid, and extracted with dichloromethane. The organic layer was dried over magnesium sulfate, filtered and concentrated. The residue was purified by preparative HPLC (Zymor Pegasus 21.2×250 mm column, 5% to 70% ethanol in heptane gradient for 13 min, 40 mL/min. HPLC t_{R}=9.48 min. 1H NMR (400 MHz, CHLOROFORM-d) δ ppm 2.25-2.85 (8H, m), 3.36 (1H, dd, J=4.1, 20 Hz), 3.56 (1 H, dd, J=11.7, 20 Hz), 4.25 (3H, s), 4.76 (1H, m), 7.02 (1H, dd, J=8.6, 2.1 Hz), 7.14 (1H, d, J=1.6 Hz), 7.53 (1H, d, J=8.6 Hz), 7.88 (1H, d, J=8.0 Hz), 8.48 (1H, d, J=8.0 Hz).

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention.

What is claimed:

1. A compound of the Formula I 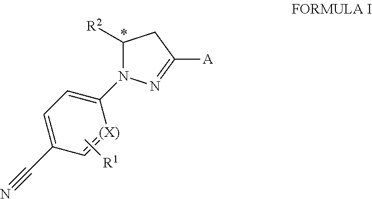
a prodrug thereof, or a pharmaceutically acceptable salt of said compound or of said prodrug;
X is N or C;
A is 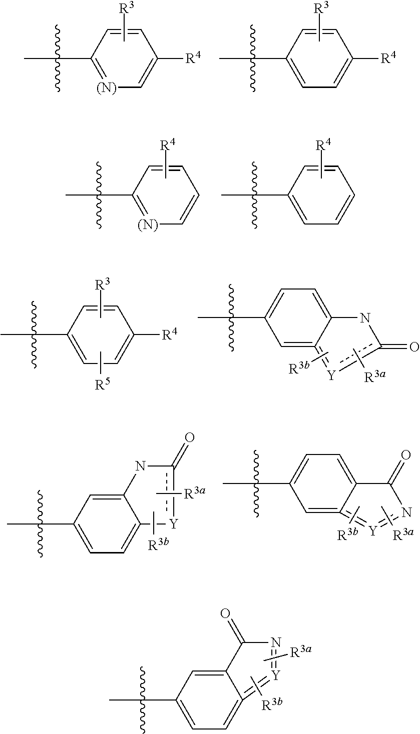
R¹ is H, halo, cyano, (C₁-C₄)alkylthio, (C₁-C₄)alkoxy or (C₁-C₄)alkyl, said (C₁-C₄)alkylthio, (C₁-C₄)alkoxy or (C₁-C₄)alkyl optionally substituted with one to nine fluoros;
R² is cyclo(C₃-C₆)alkyl, said cyclo(C₃-C₆)alkyl optionally substituted with one to four fluoros;
R³ is H, halo, hydroxyl, carboxy, carbamoyl, (C₁-C₄)alkyl, cyclo(C₃-C₆)alkyl, (C₁-C₄)alkylamino, (C₁-C₄)alkoxy, (C₁-C₄)alkylthio, (C₁-C₄)alkoxycarbonyl, (C₁-C₄)alkylsulfonyl, aminosulfonyl, (C₁-C₄)alkylsulfonylamino, (C₁-C₄)alkylcarbamoyloxy, mono-N— or di-N—,N—(C₁-C₄)alkylaminosulfonyl, mono-N— or di-N—,N—(C₁-C₄)alkylaminocarbonyl or (C₁-C₄)alkylcarbonylamino, said (C₁-C₄)alkyl optionally mono-substituted with hydroxyl, cyano, carboxy, or carbamoyl;
R⁴ is halo, hydroxyl, carboxy, carbamoyl, (C₁-C₄)alkyl, cyclo(C₃-C₆)alkyl, (C₁-C₄)alkylamino, (C₁-C₄)alkoxy, (C₁-C₄)alkylthio, (C₁-C₄)alkoxycarbonyl, (C₁-C₄)alkylsulfonyl, aminosulfonyl, (C₁-C₄)alkylsulfonylamino, (C₁-C₄)alkylcarbamoyloxy, mono-N— or di-N—,N—(C₁-C₄)alkylaminosulfonyl, mono-N— or di-N—,N—(C₁-C₄)alkylaminocarbonyl, (C₁-C₄)alkylcarbonylamino, cyano, tetrazolylcarbamoyl, (C₁-C₄)alkoxycarbonyl(C₁-C₄)alkyl, (C₁-C₄)alkoxycarbonyl, (C₁-C₄)alkylsulfonylaminocarbonyl or said (C₁-C₄)alkyl optionally mono-substituted with hydroxyl, cyano, carboxy, or carbamoyl and said mono-N— or di-N—,N—(C₁-C₄)alkylaminocarbonyl optionally mono-substituted on said (C₁-C₄)alkyl with hydroxyl, cyano or carboxy;
R⁵ is H, halo or (C₁-C₄)alkyl;
Y is a unsaturated, partially saturated or fully saturated one to three membered straight carbon chain, wherein the carbons may optionally be replaced with one or two heteroatoms selected independently from oxygen, sulfur and nitrogen, to form a five to seven membered ring; and
R^{3a} or R^{3b} is H or (C₁-C₄)alkyl,
wherein at least X is N or the A substituent contains a ring nitrogen.

2. A compound as recited in claim 1 wherein
X is C or N;
A is 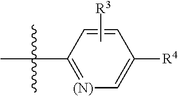
R¹ is halo, (C₁-C₆)alkyl or (C₁-C₄)alkoxy;
the pyrazoline C* is (R);
R² is cyclo(C₃-C₆)alkyl;
R³ is H, (C₁-C₄)alkylamino or (C₁-C₄)alkoxy; and
R⁴ is carboxy, carbamoyl, (C₁-C₄)alkylsulfonylaminocarbonyl or mono-N— or di-N—,N—(C₁-C₄)alkyaminocarbonyl.

3. A compound as recited in claim 2 wherein
X is C;
R¹ is in the position 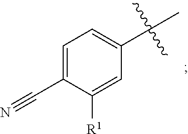
and
R³ is in the position

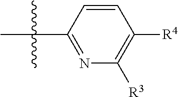

4. A compound as recited in claim 3 wherein
R¹ is halo or (C₁-C₄)alkyl;
R² is cyclopentyl;
R³ is (C₁-C₄)alkoxy; and
R⁴ is carboxy.

5. A compound as recited in claim 3 wherein
R¹ is halo or (C₁-C₄)alkyl;
R² is cyclopentyl;
R³ is (C₁-C₄)alkoxy; and
R⁴ is (C₁-C₄)alkylsulfonylaminocarbonyl

6. A compound as recited in claim 3 wherein
R¹ is halo or (C₁-C₄)alkyl;
R² is cyclopentyl;
R³ is (C₁-C₄)alkoxy; and
R⁴ is mono-N— or di-N—,N—(C₁-C₆)alkyaminocarbonyl.

7. A compound as recited in claim 1
X is N;
A is 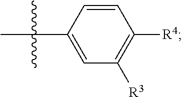
R¹ is halo, (C₁-C₆) alkyl or (C₁-C₆)alkoxy;
the pyrazoline C* is (R);
R² is cyclo(C₃-C₆)alkyl;
R³ is H, (C₁-C₄)alkylamino or (C₁-C₄)alkoxy; and
R⁴ is carboxy, carbamoyl, (C₁-C₄)alkylsulfonylaminocarbonyl or mono-N— or di-N—,N—(C₁-C₄)alkyaminocarbonyl.

8. The compound as recited in claim 7 wherein
R¹ is halo or (C₁-C₄)alkyl;
R² is cyclopentyl;
R³ is (C₁-C₄)alkoxy; and
R⁴ is carboxy, mono-N— or di-N—,N—(C₁-C₄)alkyaminocarbonyl
or (C₁-C₄)alkylsulfonylaminocarbonyl

9. The compound as recited in claim 1 wherein
X is N;
A is 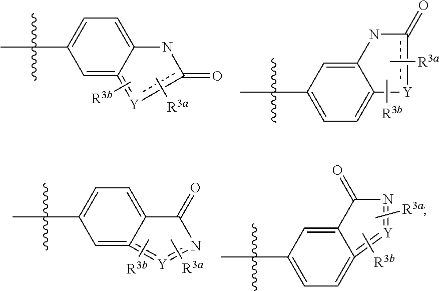
R^{3a} or R^{3b} is H or alkyl
R¹ is halo, (C₁-C₄) alkyl or (C₁-C₄)alkoxy;
the pyrazoline C* is (R); and
R² is cyclo(C₃-C₆)alkyl.

10. A compound as recited in claim 1
X is N;
A is 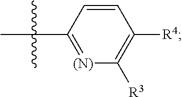
R¹ is halo, (C₁-C₄) alkyl or (C₁-C₄)alkoxy;
the pyrazoline C* is (R);
R² is cyclo(C₃-C₆)alkyl;
R³ is H, (C₁-C₄)alkylamino or (C₁-C₄)alkoxy; and
R⁴ is carboxy, carbamoyl, (C₁-C₄)alkylsulfonylaminocarbonyl or mono-N— or di-N—,N—(C₁-C₄)alkyaminocarbonyl.

11. The compound as recited in claim 10 wherein
R¹ is halo or (C₁-C₄)alkyl;
R² is cyclopentyl;
R³ is (C₁-C₄)alkoxy; and
R⁴ is carboxy, mono-N— or di-N—,N—(C₁-C₄)alkyaminocarbonyl.
or (C₁-C₄)alkylsulfonylaminocarbonyl.

12. A compound as recited in claim 1 wherein the compound is selected from
(R)-6-(1-(4-cyano-3-methylphenyl)-5-cyclopentyl-4,5-dihydro-1H-pyrazol-3-yl)-2-methoxynicotinic acid;
(R)-4-(1-(5-cyano-6-methylpyridin-2-yl)-5-cyclopentyl-4,5-dihydro-1H-pyrazol-3-yl)-2-methoxybenzoic acid;
(R)-6-(1-(5-cyano-6-methylpyridin-2-yl)-5-cyclopentyl-4,5-dihydro-1H-pyrazol-3-yl)-2-methoxynicotinic acid;
(R)-4-(1-(5-cyano-6-methylpyridin-2-yl)-5-cyclopentyl-4,5-dihydro-1H-pyrazol-3-yl)-2-ethoxybenzoic acid;
(R)-6-(1-(4-cyano-3-methylphenyl)-5-cyclopentyl-4,5-dihydro-1H-pyrazol-3-yl)-2-ethoxynicotinic acid;
(R)-6-(1-(4-cyano-3-methylphenyl)-5-cyclopentyl-4,5-dihydro-1H-pyrazol-3-yl)-2-methoxy-N-(methylsulfonyl)nicotinamide; and
(R)-6-(1-(5-cyano-6-methylpyridin-2-yl)-5-cyclopentyl-4,5-dihydro-1H-pyrazol-3-yl)-2-methoxy-N-(methylsulfonyl)nicotinamide.

13. 6-(1-(4-cyano-3-methylphenyl)-5-cyclopentyl-4,5-dihydro-1H-pyrazol-3-yl)-2-methoxynicotinic acid.

14. (R)-6-(1-(4-cyano-3-methylphenyl)-5-cyclopentyl-4,5-dihydro-1H-pyrazol-3-yl)-2-methoxynicotinic acid or a pharmaceutically acceptable salt thereof.

15. A compound having the Formula II

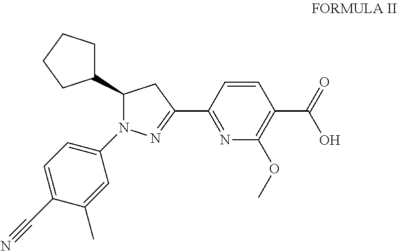

16. A method for treating cardiovascular conditions, renal conditions, liver conditions, inflammatory conditions, pain, retinopathy, neuropathy, insulinopathy, diabetic nephropathy, edema, endothelial dysfunction or baroreceptor dysfunction in a mammal (including a human being either male or female) by administering to a mammal in need of such treatment a cardiovascular conditions, renal conditions, liver conditions, inflammatory conditions, pain, retinopathy, neuropathy, insulinopathy, diabetic nephropathy, edema, endothelial dysfunction or baroreceptor dysfunction treating amount of a compound of claim 1, a prodrug thereof, or a pharmaceutically acceptable salt of said compound or of said prodrug.

17. A method as recited in claim 16 wherein diabetic nephropathy is treated.

18. A pharmaceutical composition which comprises a therapeutically effective amount of a compound of claim 1, a prodrug thereof, or a pharmaceutically acceptable salt of said compound or of said prodrug and a pharmaceutically acceptable carrier, vehicle or diluent.

19. A pharmaceutical combination composition comprising: a therapeutically effective amount of a composition comprising
a first compound, said first compound being a compound of claim 1, a prodrug thereof, or a pharmaceutically acceptable salt of said compound or of said prodrug;
a second compound, said second compound being a diuretic; and
a pharmaceutical carrier, vehicle or diluent.

20. A pharmaceutical combination composition as recited in claim 19 wherein the second compound is a torsemide.
